(19) United States
(12) Patent Application Publication
(10) Pub. No.: US 2024/0329037 A9
(43) Pub. Date: Oct. 3, 2024

(54) Rapid And Ultrasensitive Analyte Detection For Screening In Community Settings

(71) Applicant: The Trustees of the University of Pennsylvania, Philadelphia, PA (US)

(72) Inventors: Ping Wang, Wynnewood, PA (US); Zhao Li, Philadelphia, PA (US); Hui Chen, Drexel HIll, PA (US)

(21) Appl. No.: 17/199,926

(22) Filed: Mar. 12, 2021

Related U.S. Application Data
(63) Continuation-in-part of application No. PCT/US2019/050776, filed on Sep. 12, 2019.
(60) Provisional application No. 62/730,719, filed on Sep. 13, 2018.
(60) Provisional application No. 63/035,129, filed on Jun. 5, 2020.

(51) Int. Cl. G01N 33/543; G01N 33/569

(52) U.S. Cl. CPC G01N 33/54306; G01N 33/54326; G01N 33/56983; G01N 2333/165

(57) ABSTRACT

Provided are systems and methods pertaining to analyte detection, including analytes related to SARS-COV-2, such as nucleocapsid protein. The disclosed systems and methods operate by forming a complex between an analyte, a promoter tag, and an anchor and detecting a reaction product that results from the reaction between a reaction substrate and a reaction promoter of the complex. Also provided are systems and methods that allow for quantification of analyte presence by way of monitoring indicator that is displaced by a reaction associate with the analyte.

### RELATED APPLICATIONS

The present application is a continuation-in-part of international patent application no. PCT/US2019/050776, “Microbubbling And Indicator Material Displacement Systems And Methods” (filed Sep. 12, 2019), which application claims priority to and the benefit of U.S. patent application No. 62/730,719, “Point-of-Care Diagnostic Systems and Methods” (filed Sep. 13, 2018). The present application also claims priority to and the benefit of U.S. patent application No. 63/035,129, “Rapid and Ultrasensitive SARS-COV-2 Antigen Detection for Acute Infection Screening in Community Settings” (filed Jun. 5, 2020). The entireties of the foregoing applications are incorporated herein by reference for any and all purposes.

### TECHNICAL FIELD

The present disclosure relates to the field of analyte detection, in particular the detection of SARS-COV-2, as well as to the field of automated assay results.

### BACKGROUND

Sensitive analyte detection is of central importance for disease monitoring and management. Existing analyte detection platforms are, however, limited by their sensitivity and their ability to quantify results.

One significant challenge in the SARS-COV-2 pandemic is to develop sensitive and specific diagnostic methods to identify infected individuals early in the infection process, in order to promptly isolate, contact trace, and help determine clinical management course.

The current diagnostic gold standard is nucleic acid-based rRT-PCR, with various methods demonstrating variable limit of detection (LOD) from <100 to 10⁵ copies/mL. Some nucleic acid-based methods suffer from high percentage of false negatives due to limited analytical sensitivity, possibility of RNA degradation, and pre-analytical issues. On the other hand, serology tests can only detect infections 1-2 weeks post infection, and therefore are not useful to identify early infections. Sensitive, specific diagnostic methods are highly desirable to expand diagnostic capacity, decrease risks of transmission, and help determine clinical management. In this study, we use a novel approach for early/acute SARS-CoV-2 infection detection, i.e., to detect viral antigens with high sensitivity and specificity.

Detection of acute viral infections (e.g., acute HIV infections) can be achieved by detecting low concentrations of viral antigens (e.g., gag/p24, an HIV nucleocapsid protein) in body fluids. Nucleocapsid antigens are shed into the body fluids when viruses replicate in the body. Ultra-sensitive p24 assays can detect acute infections as early as the first week post infection, as well as early reactivation of infectious virus from reservoirs. The key and major challenge is to have an assay sensitive enough to detect the very low concentrations of antigens in the early phase of infection. Accordingly, there is a long-felt need in the art for sensitive analyte detection systems, in particular systems useful in POC settings. There is also a related need for analyte detection methods that are inexpensive and that can be performed in the field by individuals of varying levels of training.

### SUMMARY

Quantitating ultra-low concentrations of analytes (e.g., proteins and other biomarkers) is of key importance for early disease diagnosis and treatment. However, most current analyte detection technologies—including point-of-care (POC) assays—are limited in sensitivity. Provided here is, inter alia, a sensitive microbubbling digital assay for the quantification of analytes with a digital-readout method that can be used with only a smartphone camera. Machine learning was used to develop a related smartphone application for automated image analysis to facilitate accurate and robust counting. Using this method, post-prostatectomy surveillance of prostate specific antigen (PSA) was achieved with a detection limit (LOD) of 2.1 fM (0.060 pg mL⁻¹) and early pregnancy detection using βhCG was achieved with a detection limit of 0.034 mIU mL⁻¹ (2.84 pg mL⁻¹).

Detection of SARS-COV-2 using antigens can be an effective way to identify infections, help contact tracing and clinical management. Antigen tests using routine ELISA and lateral flow assays are often limited in analytical sensitivity, and therefore have limited utility in detecting early acute infections. In this study, we developed an ultra-sensitive SARS-COV-2 antigen test using the microbubbling digital assay previously developed in our lab. The assay achieves (as an example) a limit of detection (LOD) of 0.83 pg/mL recombinant nucleocapsid (N) antigen, and 85 copies/mL inactivated cultured SARS-CoV-2 viruses, demonstrating analytical sensitivity comparable to many rRT-PCR methods. Clinical nasopharyngeal swab testing using an EUA-approved rRT-PCR method and the microbubbling digital antigen assay showed excellent correlation. The SARS-COV-2 microbubbling digital antigen assay is an alternative diagnostic method to detect acute infections, and a very useful tool to study SARS-COV-2 antigen dynamics and its clinical implications during the course of infections, and after vaccination.

In meeting the described long-felt needs, the present disclosure provides—in one aspect—microbubbling digital assay platforms for analyte detection. The disclosed technology can utilize “express bubbling” as a signal-amplification strategy to enable single-molecule level analyte detection.

It should be understood that the disclosed technology is not limited to the POC setting, although the disclosed technology is illustrated in some cases by application to POC settings. The disclosed technology can be used in clinical, research, field, and a variety of other settings.

It should be understood that the disclosed technology is not limited to diagnostic use, although the disclosed technology is illustrated in some cases by application to diagnostics. The disclosed technology can be used in diagnostics, research, clinical trial, environmental science, forensics, drug screening, food safety and a variety of other settings.

In one embodiment, a handheld microscope (or microscope lens, as part of a smartphone accessory) is used to provide direct/digital readout. Platinum nanoparticles (PtNP) (which have good stability and excellent catalytic ability for O₂ generation) are used as the reporter for oxygen-microbubble generation. Another microfluidic design is used to prevent the coalescence of oxygen microbubbles generated from the chemical reaction catalyzed by each individual platinum nanoparticle. The specificity is conferred by antigen-antibody recognition in a sandwich immunoassay. The number of microbubbles generated correlates linearly with the concentration of the target molecules in the sample.

Using a portable microscope, one can directly read out the result without the need of extra fluorescence or luminescence devices. One can also take a picture of the result and upload to a cloud-based server to be viewed by the care provider. The device can be easily prototyped, and uses common reagents, and thus can be low-cost. The device will be able to use finger stick blood as testing samples.

By way of the disclosed use of “express bubbling” as a signal-amplification strategy to enable single-molecule level analyte detection, the existence and amount of microscope-invisible nanoparticle labels can be reflected by the microscope-visible oxygen microbubbles. Compared with fluorescence and luminescence, “express bubbling” is a much more economical and simpler strategy, without the need of sophisticated and expensive fluorescence or luminescence devices. Through use of regular microscopes or portable microscopes (or smart phones), the microbubbling technology is a significant advancement over the current state-of-the-art, transferring the “analog signal” (volume bar/pressure) to more sensitive and much more accurate “digital signal” (individual microbubbles). The microbubbling technology can provide improvements in, e.g., sensitivity/quantification and in simplification of assay procedure compatible with analyte detection, e.g., in point-of-care (POC) diagnostics.

In one aspect, the present disclosure provides methods, comprising: contacting an analyte, a promoter tag, and an anchor, the promoter tag being configured to bind to the analyte, the promoter tag further comprising a reaction promoter, the anchor being configured to bind to the analyte, the contacting being performed under conditions such that the promoter tag binds with the analyte and the anchor binds with the analyte so as to form a complex; contacting the complex with a reaction substrate so as to evolve a reaction product; and detecting at least some of the reaction product.

In another aspect, the present disclosure provides methods, comprising: contacting a plurality of first analytes, a plurality of second analytes, a plurality of first promoter tags, a plurality of second promoter tags, a plurality of first anchors, and a plurality of second anchors, the first promoter tag being configured to bind to a first analyte, the first promoter tag further comprising a reaction promoter, the first anchor being configured to bind to the first analyte, the second promoter tag being configured to bind to a second analyte, the second promoter tag further comprising a reaction promoter, the second anchor being configured to bind to the second analyte, the contacting being performed under conditions such that the first promoter tag binds with the first analyte and the first anchor binds to the analyte so as to form a first complex; the contacting being performed under conditions such that the second promoter tag binds with the second analyte and the second anchor binds to the analyte so as to form a second complex; contacting the first complex with a reaction substrate so as to evolve a first reaction product; contacting the second complex with a reaction substrate so as to evolve a second reaction product; detecting at least some of the first reaction product; detecting at least some of the second reaction product.

In a further aspect, the present disclosure provides systems, comprising: an amount of a first promoter tag, the first promoter tag being configured to bind to a first analyte, the first promoter tag further comprising a first reaction promoter, an amount of a first anchor, the first anchor being configured to bind to the first analyte and the first anchor further comprising a ferromagnetic portion; a substrate; and a gradient source configured to exert a force on the ferromagnetic portion of the first anchor.

In another aspect, the present disclosure provides methods, comprising: contacting an analyte and a promoter tag, the promoter tag being configured to bind to the analyte, the promoter tag further comprising a reaction promoter, the contacting being performed under conditions such that the promoter tag binds with the analyte so as to form a first complex; contacting the first complex with a capture tag linked to a physical substrate so as give rise to an anchored complex at an anchored complex location on the physical substrate; contacting the anchored complex with a reaction substrate so as to evolve a reaction product that advances an indicator material; and detecting an displacement of the indicator material.

The present disclosure also provides systems for detecting an analyte, comprising: a reaction chamber configured to receive one or more of a sample and a substrate; an indicator chamber in fluid communication with the reaction chamber, an amount of indicator material optionally disposed within the indicator chamber; and an indicator channel in fluid communication with the indicator chamber, the indicator channel optionally comprising one or more bends, the indicator channel configured to accommodate displaced indicator material that is displaced by evolution of a reaction product in the reaction chamber that effects displacement of the indicator material.

In one aspect, the present disclosure provides a method, comprising: contacting an analyte, a promoter tag, and an anchor, the analyte being a coronavirus nucleocapsid or a coronavirus spike, the promoter tag being configured to bind to the analyte, the promoter tag further comprising a reaction promoter, the anchor being configured to bind to the analyte, the contacting being performed under conditions such that the promoter tag binds with the analyte and the anchor binds with the analyte so as to form a complex; contacting the complex with a reaction substrate so as to evolve a reaction product, the reaction product optionally being a gas; and detecting at least some of the reaction product.

The present disclosure also provides kits, comprising: a supply of a promoter tag configured to bind specifically to an analyte, the analyte being a coronavirus nucleocapsid or a coronavirus spike; a supply of an anchor configured to bind specifically to the analyte, the anchor optionally comprising a magnetizable material, and the promoter tag comprising a material configured to evolve a gaseous product when contacted with a reaction substrate under effective conditions.

Further provided are systems, comprising: a module configured to contain a complex and the module comprising a substrate configured to receive the complex, the complex comprising: a first promoter tag, the first promoter tag bound to a coronavirus nucleocapsid or a coronavirus spike present in a sample, the first promoter tag further comprising a first reaction promoter, an amount of a first anchor, the first anchor being bound to the first analyte and the first anchor further comprising a portion sensitive to a gradient, the module being configured to engage with a detector configured to detect, within the module, a gaseous product of the reaction of the first reaction promoter with a reaction substrate.

Also provided are methods, comprising: reacting a sample comprising an amount of an analyte with (1) a promoter tag configured to bind specifically to the analyte and (2) an anchor, the reacting being performed such that the anchor and the promoter tag bind to the analyte, the analyte being a coronavirus nucleocapsid or a coronavirus spike, the anchor optionally comprising a magnetizable material, the reacting giving rise to a complex that comprises the analyte, the promoter tag, and the anchor, immobilizing the complex; contacting the complex with a reaction substrate so as to evolve a gaseous reaction product; detecting at least some of the gaseous reaction product; and correlating detected gaseous reaction product with a level of the analyte in the sample.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

For the purpose of illustrating the invention, there is shown in the drawings a form that is presently preferred; it being understood, however, that this invention is not limited to the precise arrangements and instrumentalities shown.

FIGS. 1A-1F provide a schematic of platinum nanoparticle based microbubbling assay. FIG. 1A depicts magnetic beads functionalized with capture antibodies are used to capture PtNP-labeled target molecules. FIG. 1B depicts an example microbubbling signaling strategy. Magnetic beads with/without PtNPs are loaded together with hydrogen peroxide solution into the microbubbling chip. An external magnetic field is used to settle down the magnetic beads to the bottom of the chip. Distinguishable microbubbles can be observed when magnetic bead/target molecule/PtNP sandwich complexes are present in the microwells in the microbubbling chip. FIG. 1C provides an exemplary microbubbling microchip with smart phone as readout device. FIG. 1D illustrates oxygen microbubbles entrapped in the square micro-well array, serving as a visible digital signal (not to scale). FIG. 1E provides a microscope image of the microbubbles on the microbubbling chip, with a scale bar: 200 mm. FIG. 1F provides a scanning electron micrograph of a section of the microbubbling microchip. Scale bar: 50 mm. Inset shows a platinum nanoparticle bound to a paramagnetic bead. Scale bar: 3 mm.

FIGS. 2A-2C provide kinetics of microbubble formation on a microbubbling microchip. FIG. 2A provides microscope images of the microbubbles growing on a small section of a microbubbling microchip (scale bars: 300 μm). About 25,000 Neutravidin functionalized platinum nanoparticles were incubated with biotinylated bovine serum albumin (bBSA) functionalized paramagnetic beads and loaded into the microwell array on a microbubbling microchip via magnetic field. Time were relative to the point that the magnetic field was applied. FIG. 2B provides measurements of the microbubble areas as a function of time. Each trace represents one individual microbubble. FIG. 2C provides measurements of the microbubble diameters as a function of time. Each trace represents one individual microbubble.

FIGS. 3A-3D provide quantitation of NeutrAvidin functionalized platinum nanoparticles (PtNP) with microbubbling microchips and a smart phone. Biotinylated BSA functionalized paramagnetic beads were used to load the NeutrAvidin functionalized PtNPs into the microwells; FIGS. 3A-3D provide an intrinsic sensitivity assessment of the microbubbling assay. FIG. 3A provides an example device setup for imaging microbubbles on microbubbling chip with a commercially available mobile microscope. and a smartphone. FIG. 3B provides a scheme for detecting NeutrAvidin coated PtNP using biotinylated bovine serum albumin (bBSA) functionalized magnetic beads via microbubbling. FIG. 3C provides a dose-response curve generated from experiments in FIG. 3B. The number of microbubbles correlates linearly with the amount of NeutrAvidin functionalized PtNPs. Mean standard deviation; n=3. LOD=894 PtNPs. FIG. 3D provides smartphone images of the microbubbles that appeared on the microbubbling microchips (scale bars: 1 mm) with different amounts of PtNPs.

FIGS. 4A-4E provide ultra-sensitive quantitation of prostate specific antigen (PSA) with microbubbling microchips and a smart phone. Anti-PSA monoclonal antibody functionalized paramagnetic beads were used to capture PSA molecules, which were further labelled with the NeutrAvidin functionalized PtNPs via biotinylated anti-PSA polyclonal antibodies. Smartphone images of the microbubbles that appeared on the microbubbling microchips (scale bars: 1 mm) with 100 μL of FIG. 4A—0 pg/mL PSA, FIG. 4B—0.1 pg/mL PSA, FIG. 4C—0.5 pg/mL PSA and FIG. 4D—2 pg/mL PSA. FIG. 4E illustrates that the number of microbubbles correlated linearly with the concentration of PSA. Mean±standard deviation; n=3.

FIGS. 5A-5B provide validation of the microbubbling microchips for ultra-sensitive PSA quantitation using patient serum samples. FIG. 5A provides quantitation of PSA using microbubbling microchips in serum samples with PSA undetectable with a central clinical laboratory assay (Roche Elecsys Cobas Total PSA assay, lower reportable limit 0.01 ng/ml). Mean±standard deviation; n=3. FIG. 5B provides a correlation of PSA results obtained using microbubbling microchips or a central clinical laboratory electrochemiluminescence (ECL) assay (Roche Elecsys Cobas Total PSA assay) at PSA levels >0.01 ng/mL. Mean±standard deviation for microbubbling results; n=3.

FIGS. 6A-6C provide an example image analysis smartphone application through deep learning network. FIG. 6A provides a training approach via the deep learning network. Module 1 was built to learn how to localize the specific arears of the microwell arrays. Module 2 was used to learn how to count the number of microbubbles in the specific areas. FIG. 6B provides a user interface of the microbubbling smartphone application. FIG. 6C compares readouts via the artificial intelligence (AI) approach with ImageJ-assisted manual approach for PSA detections. Mean±standard deviation for microbubbling results; n=3.

FIG. 7 provides an illustrative localization-regression machine learning network for microbubble counting on the microbubbling microchips.

FIG. 8A provides an illustrative working principle of an LFA ruler (not to scale); FIG. 8B provides a photograph of the LFA ruler. The microfluidic chip contains microchannel, distance markers, ink chamber, balance reservoir, reaction chamber and outlet. Scale bar, 1 cm.

FIGS. 9A-9B provide a correlation between number of PtNPs and ink advancement distance on LFA ruler. FIG. 9A provides ink advancement distances pushed by oxygen generated as a result of different numbers of PtNPs (0, 2.8×10⁴, 5.6×10⁴, 1.4×10⁵, and 2.8×10⁵, respectively) reacting with 30% H₂O₂. The pictures at the bottom show the density and size of bubbles in the reaction chamber after 12 min of incubation. FIG. 9B provides a linear correlation plot of ink advancement distance with number of PtNPs in 30% H₂O₂ (r²=0.99).

FIGS. 10A-10D provide a quantitation of PSA lateral flow strips with LFA ruler. Scanning electron microscope images of the test zone pads from positive strip. FIG. 10A and blank strip FIG. 10B, respectively. The green arrow identifies PtNPs in the cavities of nitrocellulose membrane. FIG. 10C provides ink advancement distances in the LFA ruler with different PSA concentrations (0, 1, 2, 4, 8, and 12 ng/ml, respectively). FIG. 10D provides a linear correlation between ink advancement distance and PSA concentration, tested in triplicates (r²=0.99).

FIGS. 11A-11B provide a validation of the LFA ruler against clinical gold standard PSA assay. FIG. 11A provides a histogram of the clinical serum sample test results generated by the LFA ruler (mean±standard error) and the ECLIA assay (Roche Elecsys Cobas Total PSA). Two dashed lines represent the clinical cutoffs for PSA, 4 ng/ml and 10 ng/mL, respectively. FIG. 11B illustrates a linear relationship between the LFA ruler and standard clinical results with an r² value of 0.92. (r²=0.95 in the inset, for PSA concentrations below 12 ng/ml).

FIG. 12A provides a microscope image of a 3-μm-thick layer of low-permeability Parylene C (PC) membrane deposited on the surface of the LFA ruler. Scale bar, 50 μm. FIG. 12B provides ink advancement distances in different LFA rulers with/without PC membrane, pushed by oxygen generated as a result of different numbers of PtNPs (0, 5.6×10⁴; 0, 5.6×10⁴, respectively) reacting with 30% H₂O₂. Illustrations on both sides are the enlarged views of the black dotted rectangles. Under a certain angle of illumination, the label on the device without PC membrane is gray; the label on the device with PC membrane is colored.

FIG. 13 provides an exemplary plot of time-dependent ink advancement distances. The number of platinum nanoparticles is 0, 2.8×10⁴, 5.6×10⁴, 1.4×10⁵, and 2.8×10⁵, respectively.

FIG. 14A provides images of LFA strips. There is no difference in color between the blank strip (0 ng/mL PSA) and the positive strip (8 ng/mL PSA) with the naked eye. FIG. 14B provides ink advancement distances of the test/control zone from the blank strip (0 ng/mL PSA) and the positive strip (8 ng/mL PSA) is significantly different in the LFA rulers.

FIGS. 15A-15C provide an illustration of an application via machine learning for counting microbubbles in smartphone images. FIG. 15A show that a localization network can take the raw images as input, and outputs the location of the microwell array region. The cropped images are fed into the regression network that outputs the bubble counts. FIG. 15B shows an exemplary user interface of the mobile application. FIG. 15C illustrates that the readouts via the CNN approach correlated well with ImageJ-assisted manual approach.

FIGS. 16A-16C provide a demonstration of ultra-sensitive quantitation of prostate specific antigen (PSA) with microbubbling assay for prostate cancer post-prostatectomy surveillance. Anti-PSA monoclonal antibody functionalized paramagnetic beads were used to capture PSA molecules, which were further labelled with the NeutrAvidin functionalized PtNPs via biotinylated anti-PSA polyclonal antibodies. FIG. 16A provides an example dose-response curve of microbubbling PSA assay. FIG. 16B illustrates that in the dynamic range, the number of microbubbles correlated linearly with the concentration of PSA. Mean±standard deviation; n=4. LOD=0.060 pg/mL (2.1 fM). FIG. 16C demonstrates a validation of the microbubbling assay for ultra-sensitive PSA quantitation using patient serum samples. Comparison of PSA results obtained using microbubbling assay with a central clinical laboratory electrochemiluminescence (ECL) assay (Roche Elecsys Cobas Total PSA assay) Mean±standard deviation for microbubbling results; n=3.

FIG. 17 provides an illustration of the process of the microbubbling chip fabrication. As shown, standard soft lithography is used to fabricate polydimethylsiloxane (PDMS) sheet with micro well array from an SU-8 mold. The PDMS sheet is transferred on a glass slide with the feature side facing up and a PDMS chamber placed on top. Finally, a layer of parylene C is coated on top of the chip via physical vapor deposition (PVD) to prevent diffusion of oxygen into PDMS.

FIGS. 18A-18B demonstrate that microbubbling can be microwell-dependent on the microchip. FIG. 18A provides that a microbubbling microchip contains a central microwell array area (3 mm×3 mm) surrounded by plain area (no microwells). External magnetic field deposits PtNP bonded magnetic beads in both the microwell area and the plain area. FIG. 18B shows microbubbles found only in the microwell area.

FIG. 19A-19B demonstrates that microbubbles are found in the same microwells repeatedly after replacing the H2O2 solution. FIG. 19A shows a solution containing 1200 NeutrAvidin coated PtNPs was loaded in a microbubbling microchip via bBSA coated magnetic beads, and 5 microbubbles were observed at different positions on the chip. FIG. 19B shows that after replacing the top bulk H2O2 solution with fresh H2O2 solution, 3 microbubbles were regenerated at the same positions of the chip with sizes comparable to the previous microbubbles. Two microbubbles were lost, probably because PtNPs in these two wells were washed away during the changing of H2O2 solution.

FIGS. 20A-20B show microbubble growth. FIG. 20A shows the growth of microbubbles under different ambient temperatures. Each trace represents the growth of one individual microbubble. All assay reagents were equilibrated to targeted ambient temperatures before experiment, and the growth of microbubbles were recorded under a portable microscope. FIG. 20B compares the growth speeds of microbubbles under different ambient temperatures. Mean±standard deviation; n=3.

FIGS. 21A-21B show an optimization of the amount of magnetic beads used in the microbubbling assay. FIG. 21A shows different amounts of biotinylated BSA coated magnetic beads were used to load various amounts of NeutrAvidin coated PtNPs. FIG. 21B shows that the number of microbubbles generated were plotted against number of PtNPs, at different amount of magnetic beads. To balance signal intensity and variation, 2×105 magnetic beads were chosen for subsequent experiments.

FIG. 22 provides scanning electron micrograph images of microwells loaded with magnetic beads under assay conditions (microwell number: magnetic bead number, 10,000:200,000).

FIGS. 23A-23B provide optimization of the concentration of H2O2 solution used in the microbubbling assay. FIG. 23A shows different amounts of NeutrAvidin coated PtNPs were loaded with 2×10⁵ biotinylated BSA coated magnetic beads and further incubated with different concentrations of H2O2. FIG. 23B shows that the number of microbubbles generated were plotted against PtNPs concentrations at various concentrations of H2O2. 30% H2O2 was chosen for subsequent experiments due to maximum signal intensity.

FIGS. 24A-24B provide a design of the CNN. FIG. 24A shows a localization-regression machine learning network for microbubble counting on the microchips. FIG. 24B shows that the smart phone application and CNN model is robust to variations in illumination conditions and microbubble sizes and overlapping cases.

FIGS. 25A-25B provide an optimization of the concentration of NeutrAvidin coated PtNP used in the microbubbling assay for PSA detection. FIG. 25A provides an example assay design. FIG. 25B provides smartphone images of microbubbles at different concentrations of PtNPs at blank or 1 pg/mL PSA. PtNP slurry with a concentration of 0.78×107/mL was chosen for subsequent experiments for optimal signal/noise ratio.

FIG. 26 provides smartphone images of the microbubbles that appeared on the microbubbling microchips (scale bars: 1 mm) with 100 μL of standard solutions of different PSA concentrations.

FIG. 27 provides a comparison of PSA results obtained using the microbubbling assay and Simoa digital ELISA assay (QUANTERIX, Simoa HD-1 ANALYZER).

FIGS. 28A-28H provide a quantitation of beta subunit human chorionic gonadotropin (βhCG) with the microbubbling assay and a smart phone. Anti-βhCG antibody functionalized paramagnetic beads were used to capture βhCG molecules which were further labelled with PtNPs via detection antibodies. Smartphone images of the microbubbles that appeared on the microbubbling microchips (scale bars: 1 mm) with 0 (FIG. 28A), 0.94 pg/mL (FIG. 28B), 1.88 pg/mL (FIG. 28C), 3.75 pg/mL (FIG. 28D), 7.50 pg/mL (FIG. 28E), 15.00 pg/mL (FIG. 28F), and 30 pg/mL (FIG. 28G) βhCG. FIG. 28H shows that the number of microbubbles correlates linearly with the concentration of βhCG. Mean±standard deviation; n=3. The LOD was calculated by extrapolating the concentration of βhCG at background plus 3 standard deviations of the background.

FIG. 29 provides the coefficient(s) of variations of the microbubbling assay for PSA quantitation.

FIGS. 30A-30B provide an example design of the microbubbling digital assay for the detection of SARS-COV-2 Nucleocapsid Protein (N-Protein). FIG. 30A Schematic of microbubbling digital N antigen assay. FIG. 30B Dose-response curve and Smartphone imaging results. The number of microbubbles correlates linearly with the amount of N-Protein. Mean±standard deviation; n=3. LOD-0.83 pg/mL.

FIG. 31 provides a limit of detection of the microbubbling digital N antigen assay for the detection of inactivated SARS-Cov-2 cultured virus. The number of microbubbles correlates linearly with the amount of inactivated SARS-Cov-2 viruses within the (red) dotted range. Mean±standard deviation; n=3. LOD=0.085 copies/μL.

FIG. 32 provides examples of microbubble images from testing clinical nasopharyngeal swab samples using the microbubbling digital assay. “+” and “−” indicate results by the Cepheid GeneXpert™ SARS-COV-2 rRT-PCR method.

### DETAILED DESCRIPTION OF ILLUSTRATIVE EMBODIMENTS

The present disclosure can be understood more readily by reference to the following detailed description taken in connection with the accompanying figures and examples, which form a part of this disclosure. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable, and it should be understood that steps can be performed in any order. Any documents cited herein are incorporated by reference in their entireties for any and all purposes.

It is to be appreciated that certain features of the invention which are, for clarity, described herein in the context of separate embodiments, can also be provided in combination in a single embodiment. Conversely, various features of the invention that are, for brevity, described in the context of a single embodiment, can also be provided separately or in any subcombination. Further, reference to values stated in ranges include each and every value within that range. In addition, the term “comprising” should be understood as having its standard, open-ended meaning, but also as encompassing “consisting” as well. For example, a device that comprises Part A and Part B can include parts in addition to Part A and Part B, but can also be formed only from Part A and Part B.

### Illustrative Disclosure—Bubbling

Quantitating ultra-low concentrations of protein analytes is critical for early disease diagnosis and treatment. However, most current analyte detection approaches-including point-of-care (POC) assays-are limited in sensitivity to meet this clinical need.

Provided here is, inter alia, a sensitive microbubbling digital assay readout method toward quantitation of protein analytes requiring only bright-field smartphone imaging. Picolitre-sized microwells together with platinum nanoparticle labels enable the discrete “visualization” of protein molecules via immobilized-microbubbling with smartphone. One can also use computer vision and machine learning to develop an automated image analysis smartphone application to facilitate accurate and robust counting.

Using this method, post-prostatectomy surveillance of prostate specific antigen (PSA) can be achieved with a detection limit of 2.1 fM (0.060 pg/mL), and early pregnancy detection using βhCG with a detection limit of 0.034 mIU/mL (2.84 pg/mL). The results are further validated using clinical serum samples against clinical and research assays.

The present technology is applicable to a variety of settings. One such setting is POC venues, where POC protein assays are used to provide clinically actionable results of protein analytes at the point-of-use, requiring no sample processing or analysis from a remote clinical central laboratory, and meet the increasing demand of patient-centered health care. They connect the testing and the consultation process for patients and therefore avoid multiple visits to healthcare providers otherwise required by centralized testing.

Existing POC protein assays, e.g., lateral flow assays, are limited in sensitivity and precision. On the other hand, the beginning of the 21st century has witnessed significant advances in pursuit of ultra-high sensitivity for protein analyte detections in the research settings. In 2010, the single-molecule enzyme-linked immunosorbent assay (digital ELISA) first introduced the revolutionary “digital assay” concept into the field of protein detection. In digital ELISA, individual protein molecules were directly counted via the discrete fluorescent digital signals, achieving PCR-like sensitivity for protein detection. Although sensors in digital assays only need to distinguish between positive and negative signals, digital ELISA mainly relies on fluorescent labels and requires sophisticated and nonportable laboratory based high-resolution fluorescence microscopy system.

Direct visualization as a readout method can be more suitable than fluorescence (e.g., in the laboratory setting and even in a POC setting), since no extra optical system is needed to filter excitation and emission light. Replacing the fluorescent labels in digital ELISA with submillimeter-sized bright field visible labels (such as microparticles) can permit direct visualization. However, unlike nanosized labels, it is challenging to directly label discrete biomolecules with individual microscope-visible particles. For example, some have tried dipole-dipole assisted interactions and well controlled microfluidic drag force to label protein molecules with 2.8 μm magnetic beads. Some have used 30 nm gold nanoparticles to label the protein molecules and then used nanoparticle-promoted reduction to increase the size of the gold nanoparticle to amplify the signal.

The disclosed technology facilitates the translation of ultra-high sensitivity assay to clinical use by introducing a new signaling strategy: immobilized-microbubbling, one distinguishable physical transformation process involving quick volume amplification with minimum mass increase. One can use microbubbling as a “bridge” to connect the “invisible” nano-world to the “visible” micro-world.

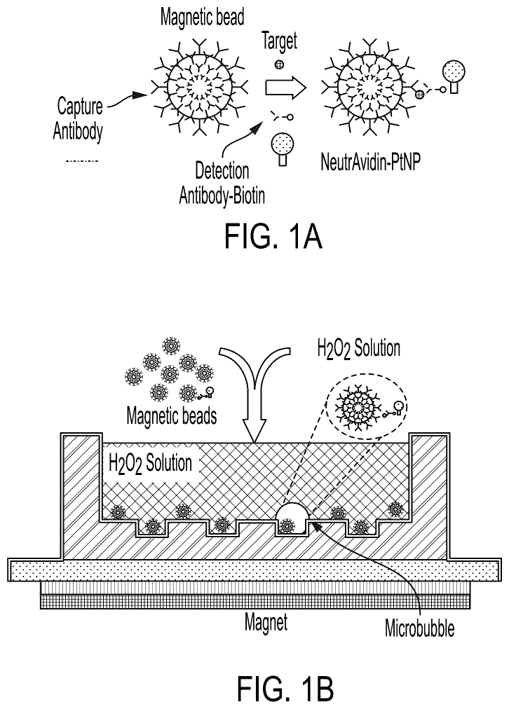

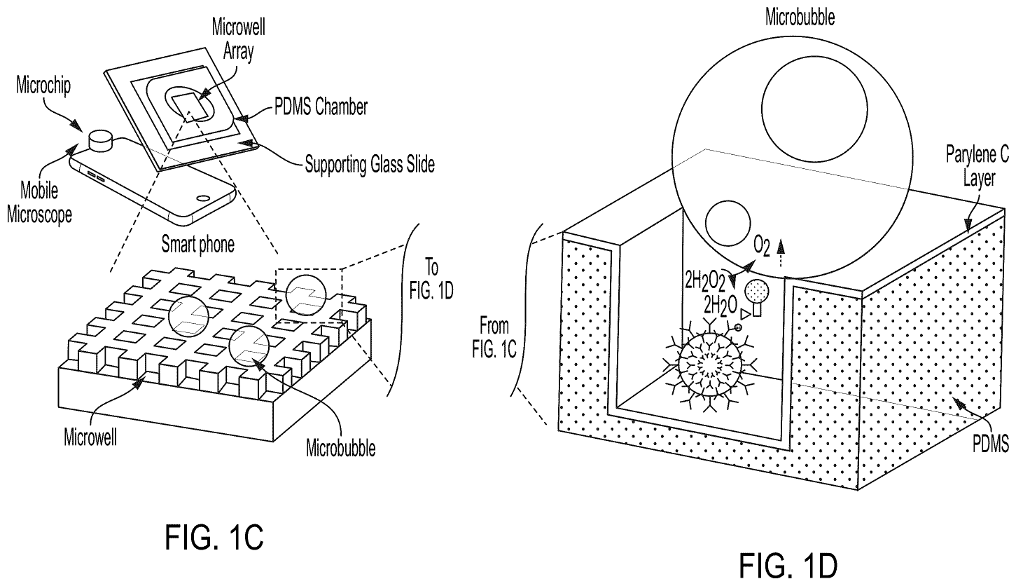

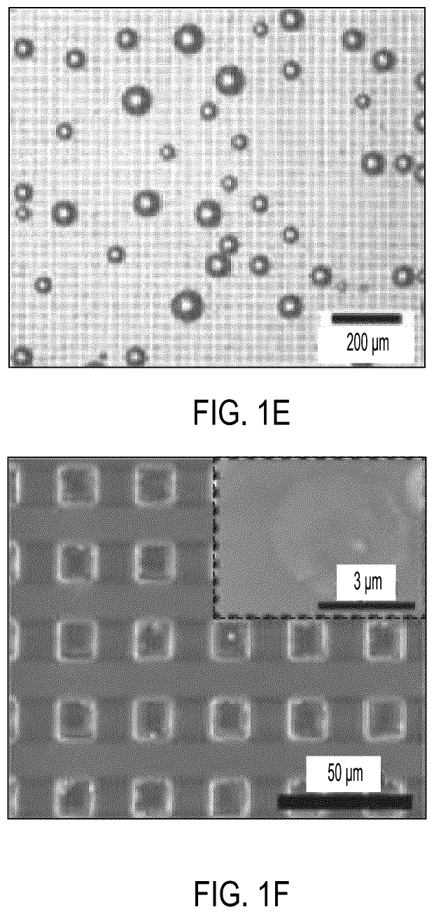

In one disclosed approach, one can use platinum nanoparticle (PtNP) catalyzed immobilized submillimeter-sized microbubbles to visualize protein molecules. This first of its kind application can be termed a platinum nanoparticle based microbubbling assay, aiming for the ultra-sensitive detection of protein analytes with smartphone enabled bright field imaging as a new readout strategy for use, as shown in FIG. 1. (It should be understood that although this disclosure utilizes platinum materials as illustrative, the present disclosure is not limited to platinum materials, and other materials—e.g., silver—can be used in place of or even with platinum.)

In the microbubbling digital assay, target protein molecules are captured by the capture antibodies on paramagnetic microbeads (˜2.7 μm), and the bound complexes are further labelled with PtNPs. The sandwich complexes are loaded together with hydrogen peroxide solution into an array of square-shaped microwells (14 μm×14 μm, 7 μm depth, 100×100, 3 mm×3 mm) on the microbubbling microchip via external magnetic field.

Microbubbles form as a result of the accumulation of oxygen catalyzed by PtNPs in the microwells, which can be easily seen with mobile microscope (e.g., 9×) using smart phone camera. When the number of sandwich complexes to the number of microwells is below 1:1, the percentage of sandwich complexes loaded microwells follows Poisson distribution, which indicates that the microwells are loaded with a single sandwich complex or none. Therefore, the “yes/no” state of microbubbling digitally represents the “yes/no” state of the existence of a sandwich complex in the microwell.

Compared with the analogue signals from PtNPs, such as the ensemble volume or pressure change caused by the PtNPs-catalyzed oxygen generation, the digital (“yes/no” state) signals in microbubbling assay are less influenced by the environmental temperature and pressure variations. Therefore, the background noise of microbubbling assay is much lower, resulting in the dramatic increase in sensitivity.

Furthermore, like the gold nanoparticles used in lateral flow immunoassay, the PtNPs used in microbubbling assay are also stable for long-term storage and transportation. To provide a precise and user-friendly readout, also provided is a machine learning based automated image analysis smartphone application to count the number of microbubbles under a variety of imaging conditions. Exemplary microbubbling assays are used to quantitate two model proteins: prostate-specific antigen (PSA) for post-prostatectomy prostate cancer surveillance and ß subunit of human chorionic gonadotropin (βhCG) for early pregnancy detection, as two clinical application examples.

The microbubbling microchip consists of three major parts as shown in FIG. 1 C: 1) the sample chamber, 2) the microarray layer and 3) the supporting glass slide. The size of the microarray is designed to be 3 mm×3 mm to fit the field of view of the mobile imaging system. The microwell was designed in square shape to be easily distinguished from the round microbubbles, though this is not a requirement.

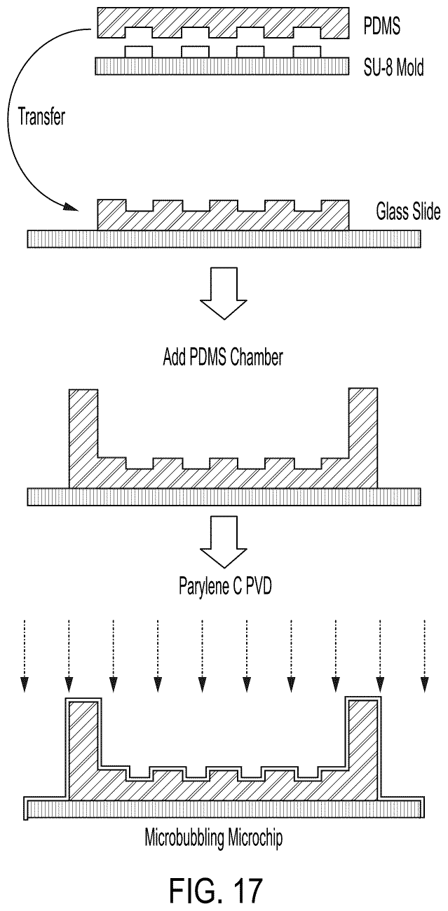

To fabricate the microwells, one can use standard soft lithography to make the polydimethylsiloxane (PDMS) microwells, which were further coated with a 3 μm thick layer of perylene C via physical vapor deposition (PVD) to prevent the diffusion of oxygen into PDMS, as shown in FIG. 17. The microbubbling assay procedure is shown in FIGS. 1A and 1B. Magnetic beads, functionalized with capture antibodies, are used to capture target molecules, which are further labeled with PtNPs via detection antibodies. All the magnetic beads with/without PtNPs are loaded into the chamber of the microbubbling chip together with hydrogen peroxide solution. An external magnetic field (by placing a magnet under the microbubbling chip for 1 min) is used to settle down all the magnetic beads to the bottom of the microbubbling chip.

Distinguishable microbubbles can be observed in the microwells of the chip, when magnetic bead/target molecule/PtNP sandwich complexes are present in the corresponding microwells.

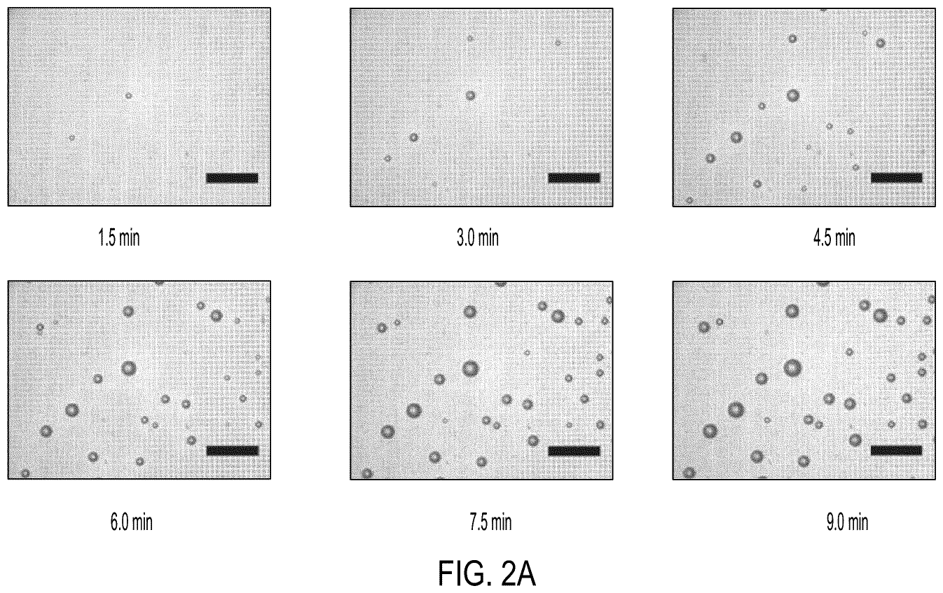

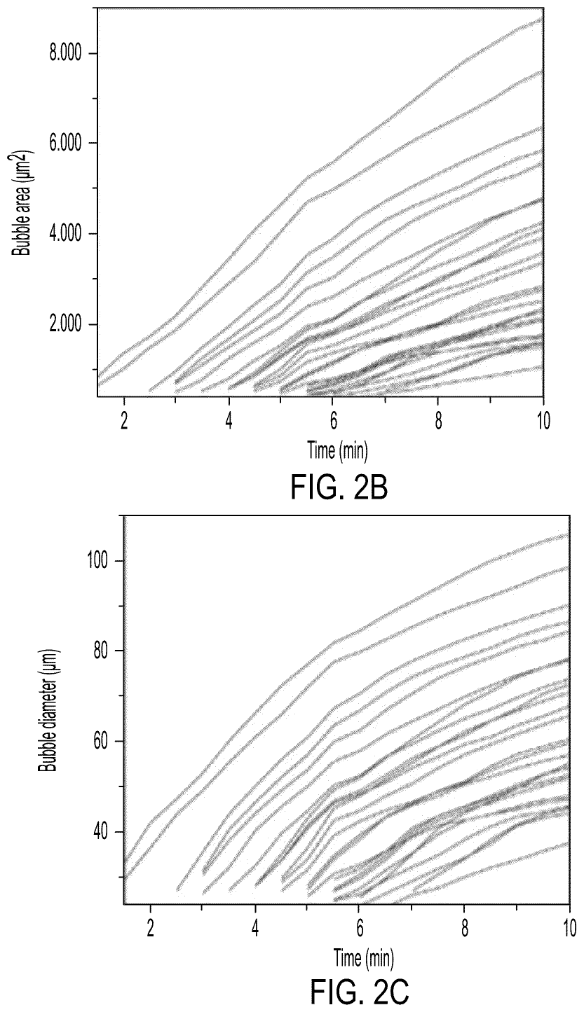

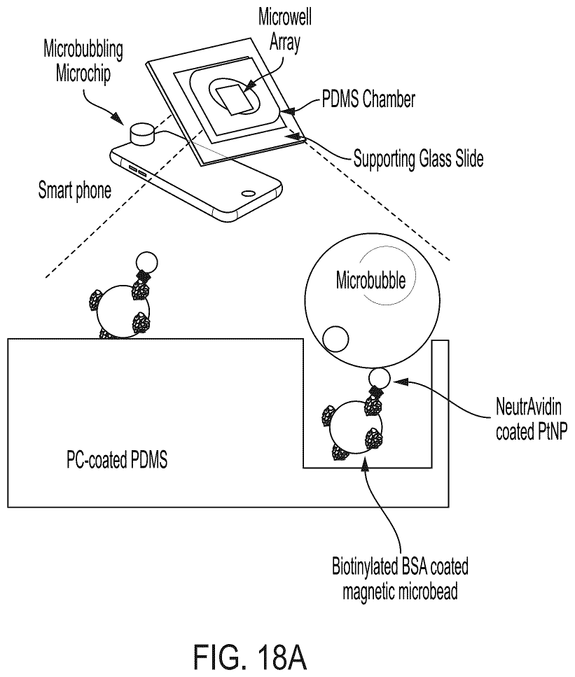

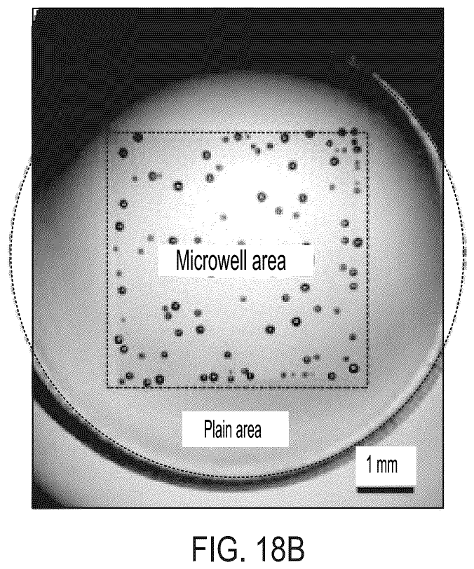

It was found that the formation of microbubbles is microwell-dependent. As shown in FIGS. 18A-18B, microbubbles were only found in the microwell area but not in other area without microwells. One can hypothesize that the growth of the microbubbles is facilitated by the rapid local oxygen accumulation in the microwells. To assess the kinetics of the microbubbling process on the microchip, biotinylated bovine serum albumin (bBSA) coated paramagnetic microbeads were used to capture NeutrAvidin functionalized PtNPs, and then loaded the beads together with hydrogen peroxide solution into the microwell array on a microbubbling microchip via external magnetic field. As shown in FIG. 2A, the microbubbles increased quickly after the beads were loaded. FIG. 2B provides measurements of the microbubble areas as a function of time. Each trace represents one individual microbubble. FIG. 2C provides measurements of the microbubble diameters as a function of time. Each trace represents one individual microbubble.

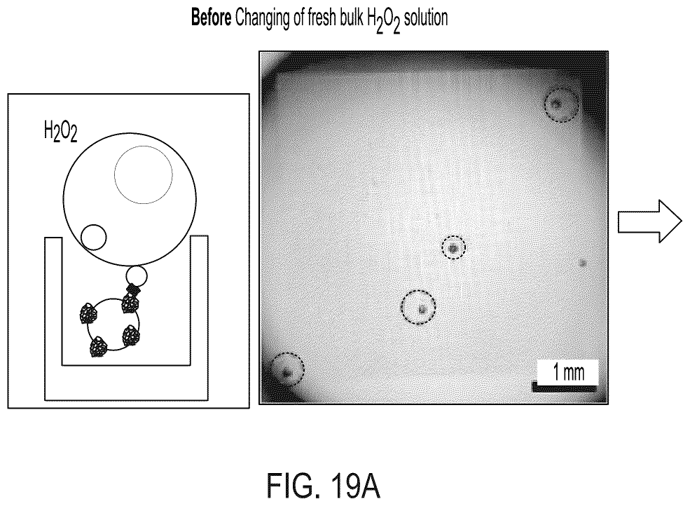

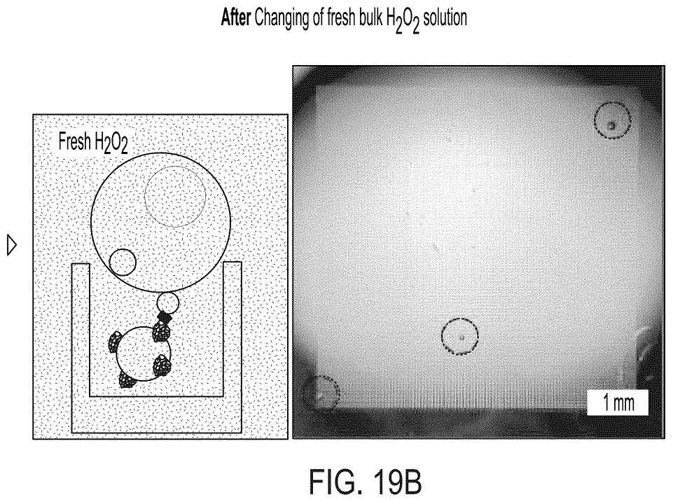

All the microbubbles became visible under conventional microscope within 8 min. All the microbubbles originated from the centers of corresponding microwells and kept growing with these microwells as centers, indicating the growth of the microbubbles were powered by the gas-generating reaction catalyzed by the PtNPs trapped in the corresponding microwells. This was further confirmed by the fact that after replacing solution in the microchip with fresh hydrogen peroxide solution, new bubbles appeared again in the exact same microwells (FIGS. 19A-19B).

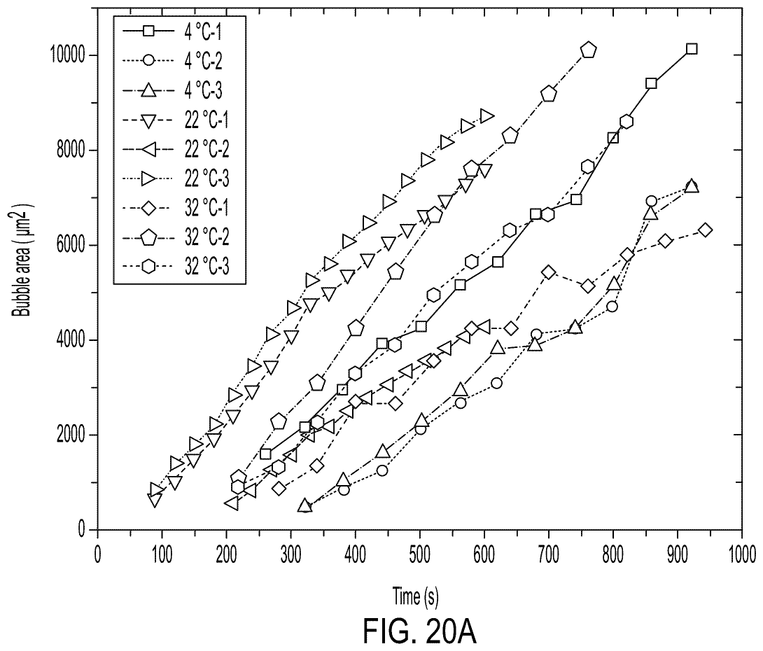

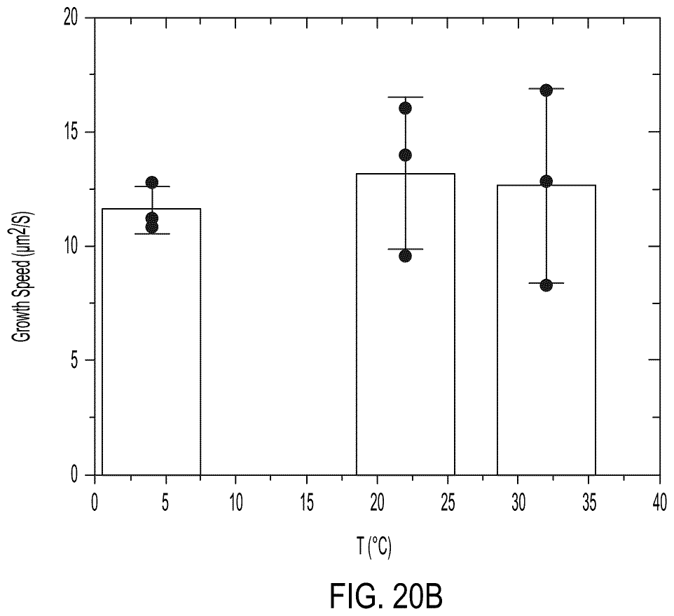

As shown in FIGS. 2B-2C, microbubbles started appearing at different time points, indicating the increase of local oxygen concentrations varied in different microwells. Without being bound to any particular theory, this may be due to the variations in number, size, mass transfer, shape, and surface coverage of the PtNPs in these bubble-generating microwells. Ambient temperature does not significantly affect the kinetics of bubble growth, as shown in FIGS. 20A-20B.

One can hypothesize that the formation of microbubbles in microbubbling assay is a composite chemical-physical phenomenon dependent on the balance between the local generation and the diffusion (into the bulk of the liquid phase) of oxygen molecules. When local speed of oxygen generation surpasses the speed of oxygen diffusion into the bulk liquid phase, microbubbles form and grow. This is supported by the finding that microbubbles were only found in microwells where the diffusion of oxygen molecules into bulk liquid phase was restricted by the walls of microwells. When temperature increases, both the generation and the diffusion speed of oxygen molecules increase, resulting in the overall growth speed of microbubbles relatively constant in the range from 4 deg. C to 32 deg. C.

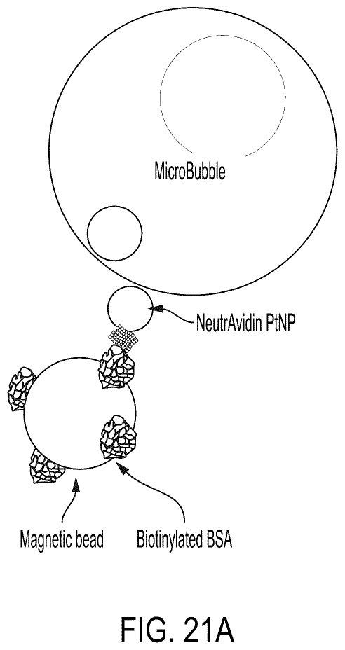

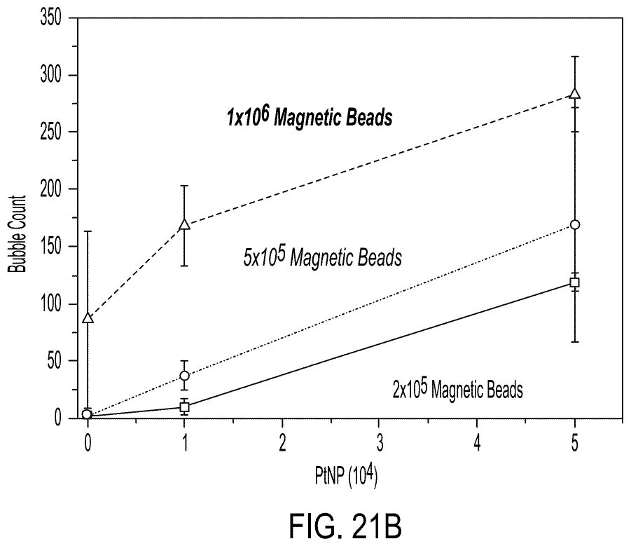

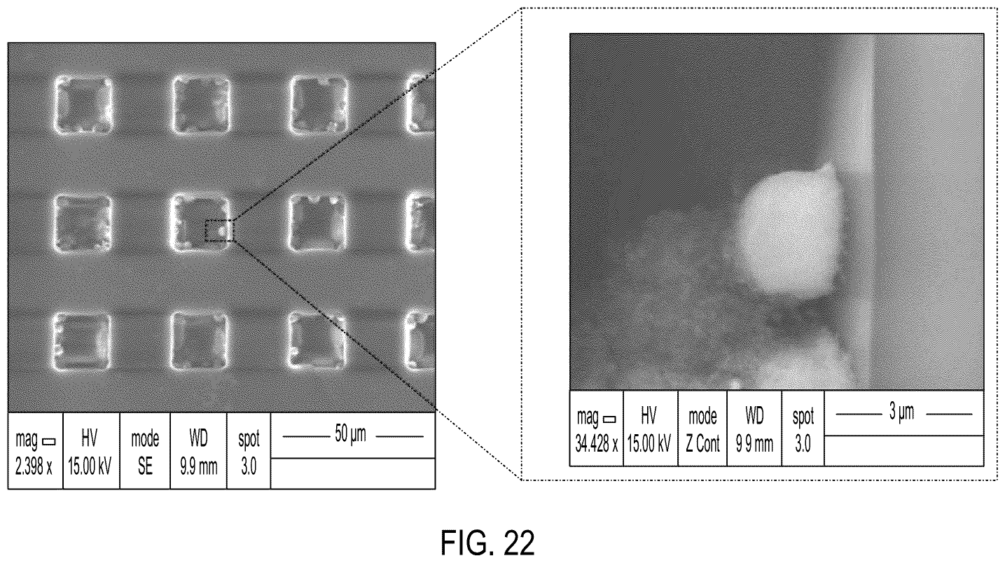

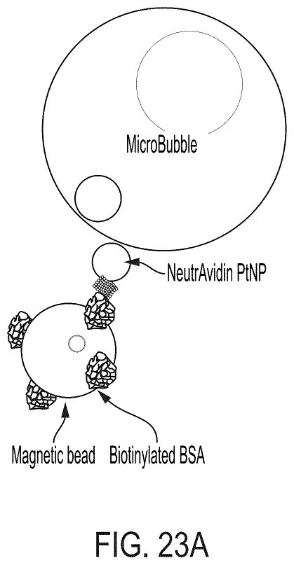

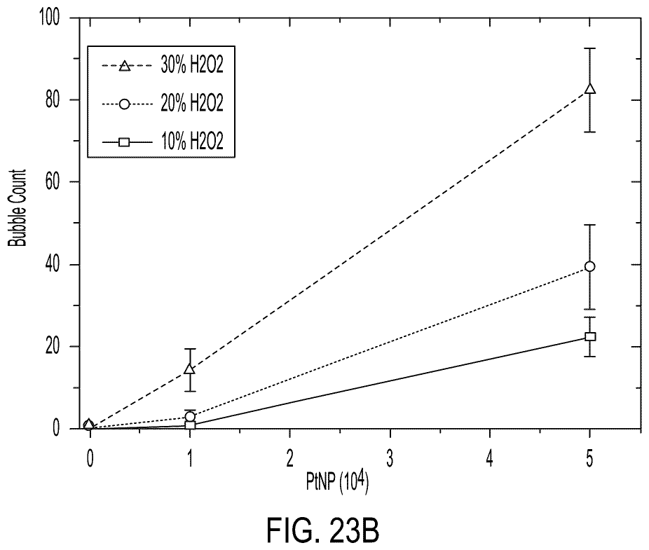

To explore the intrinsic sensitivity of the microbubbling assay, one can optimize the amount of magnetic beads (FIGS. 21A-21B and 22) and concentration of hydrogen peroxide solution (FIG. 23A-23B). A ratio between the number of magnetic beads (˜200,000) and the number of microwells (10,000) was used in the assay to make sure most of the microwells are loaded with magnetic beads in each measurement (FIG. 22).

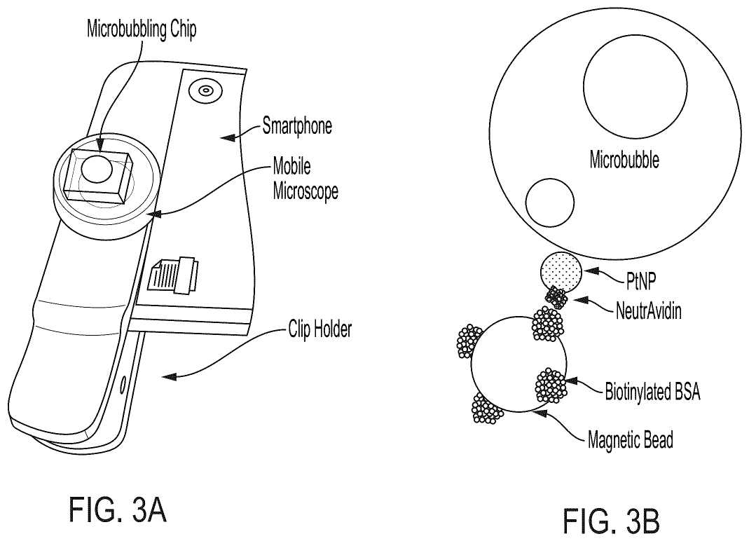

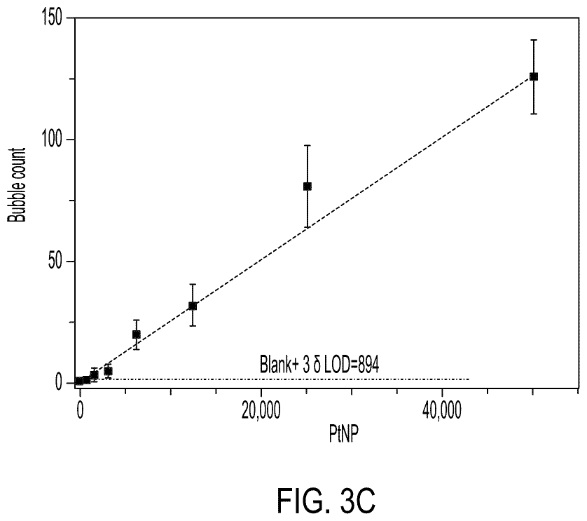

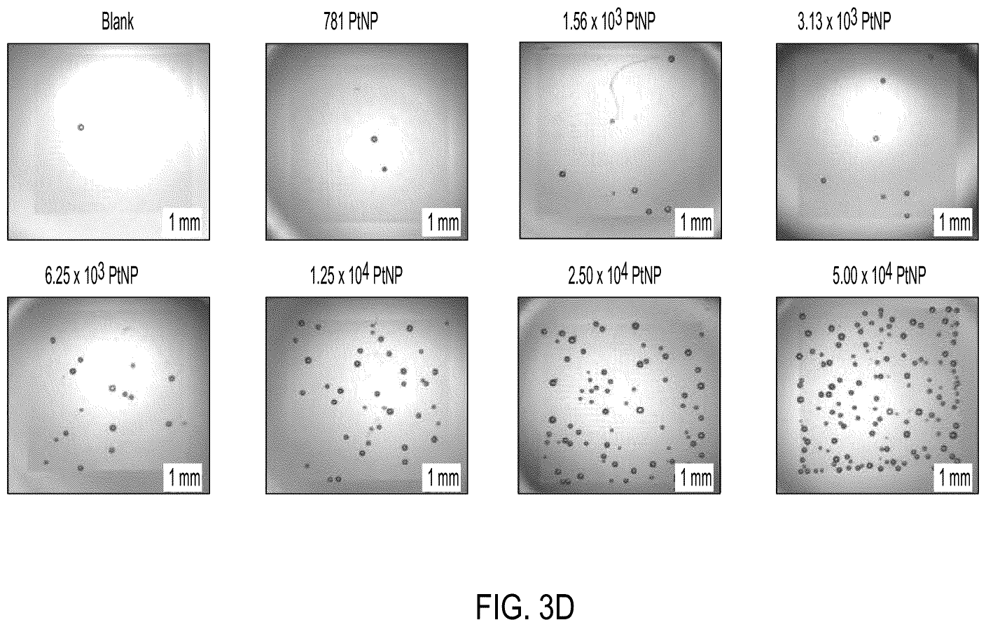

FIG. 3A provides an example device setup for imaging microbubbles on microbubbling chip with a commercially available mobile microscope. and a smartphone. FIG. 3B provides a scheme for detecting NeutrAvidin coated PtNP using biotinylated bovine serum albumin (bBSA) functionalized magnetic beads via microbubbling. FIG. 3C provides a dose-response curve generated from experiments in FIG. 3B. The number of microbubbles correlates linearly with the amount of NeutrAvidin functionalized PtNPs. Mean standard deviation; n=3. LOD=894 PtNPs. FIG. 3D provides smartphone images of the microbubbles that appeared on the microbubbling microchips (scale bars: 1 mm) with different amounts of PtNPs. BBSA coated paramagnetic microbeads were used to capture a range of numbers of NeutrAvidin functionalized PtNPs, and then loaded the beads together with hydrogen peroxide solution into the microwell arrays on microbubbling microchips via external magnetic field. After 8 minutes, the microbubbles on the microbubbling microchips were imaged using an iphone 6 plus together with a commercial mobile microscope (9×). As shown in FIG. 3C, the number of microbubbles correlated linearly with the number of PtNPs, with a limit of detection (LOD) of 894 PtNPs. The LOD was calculated by extrapolating the amount of PtNPs at background plus 3 standard deviations of the background.

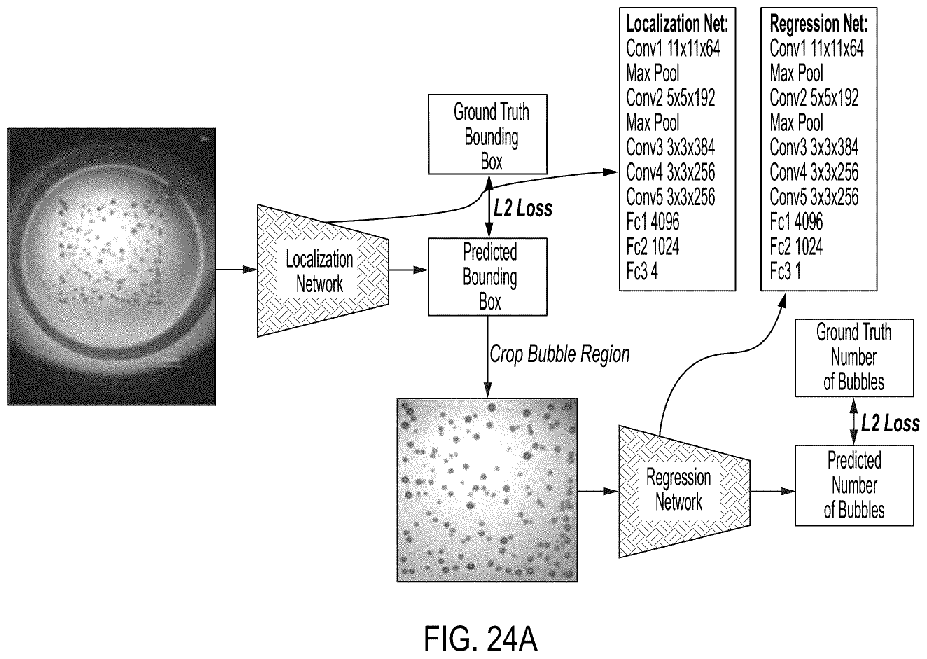

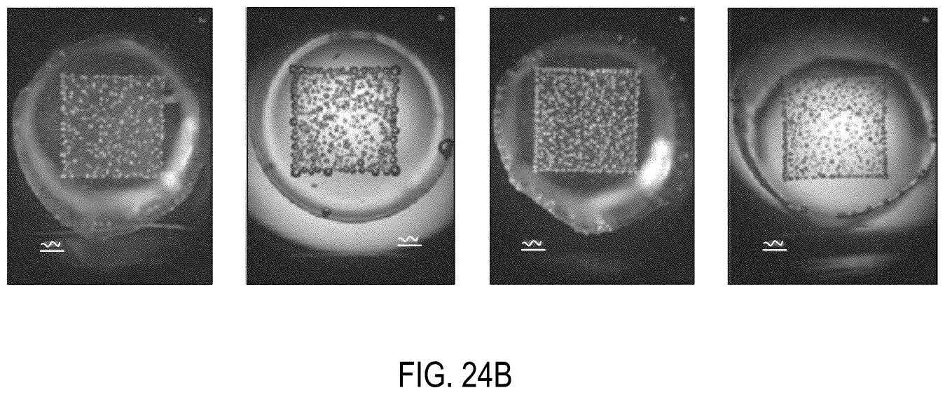

Owing to their unique light scattering properties and shape, the microbubbles can be easily distinguished in the images by human eye or a conventional image processing algorithm. But the color and brightness of microbubbles can vary significantly as shown in FIGS. 24A-24B, when images are taken under a variety of illumination conditions, which can occur in some settings.

To increase the robustness and accuracy of the image processing algorithm for bubble counting, one can utilize a convolutional neural network (CNN) to identify and count the number of microbubbles in the images. CNN has been utilized in the past several years in vision tasks, such as image recognition, semantic segmentation and object detection.

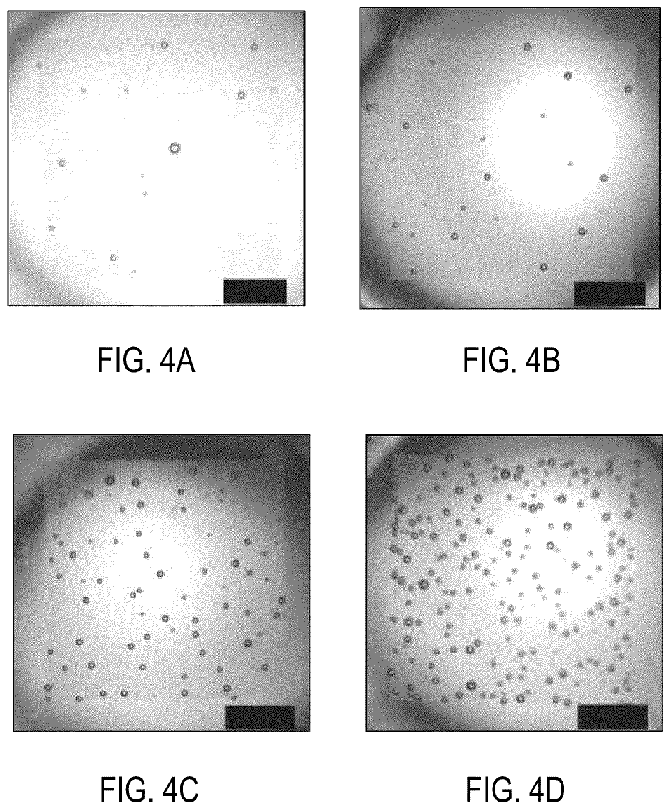

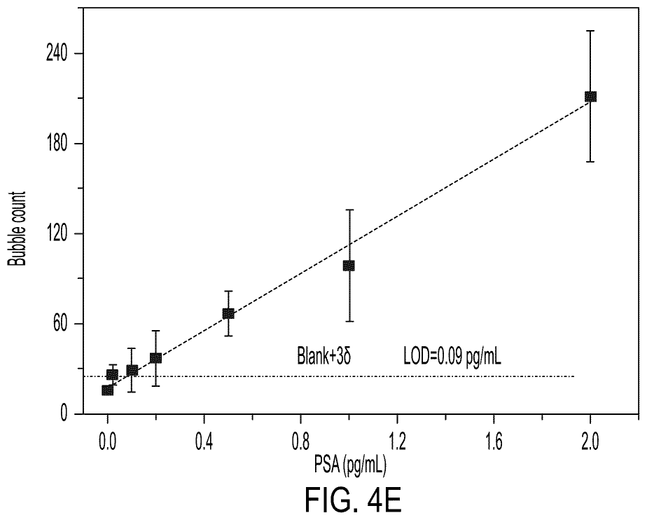

The main advantage of the CNN architecture is that it can learn expressive feature representation with high-level semantics for specific tasks, and it is robust to poor image quality due to less-than-ideal imaging conditions. A smartphone application for microbubbling via the CNN was developed, as shown in FIG. 4A. After training the algorithm with 493 images (detailed training network and process in the supporting information, FIGS. 24A-24B), the application can successfully identify the boundaries of the microarray areas and count the microbubbles in seconds.

The application is robust to variations in illumination condition and microbubble size and overlapping cases (FIGS. 24A-24B). Examples of smartphone application interfaces are shown in FIGS. 4A-4DB. As shown in FIG. 4E, the microbubble counts of 22 test images via the CNN correlate well with ImageJ-assisted manual counts.

As one application of the disclosed technology, ultrasensitive PSA assessment in the post-prostatectomy surveillance of prostate cancer patients is useful as a means of risk stratification and counselling of patients on prognosis and treatment decisions. Early detection of recurrence offers the possibility of early salvage therapy given at a lower cancer burden and a wider time window for cure. Postoperative PSA >0.073 ng/ml at day 30 increased the risk of biochemical recurrence in the presence of positive surgical margins (PSM) after radical prostatectomy, demonstrating that ultrasensitive PSA can aid risk stratification in patients with PSM. Patients not likely to experience biochemical recurrence can be spared from the toxicity of immediate adjuvant radiotherapy.

An ultrasensitive PSA detection scheme can allow urologists to test patients in their offices during follow-up visits after surgery, or eventually allow a telemedicine approach in which patients monitor themselves at home and transmit results to urologists. This would shorten the detection time of recurrence, enable immediate discussion of the result as preferred by the patients and administration of salvage therapy if necessary. Studies have reported salvage radiation therapy given soon after ultra-PSA is detectable substantially reduces the risk of relapse and metastasis.

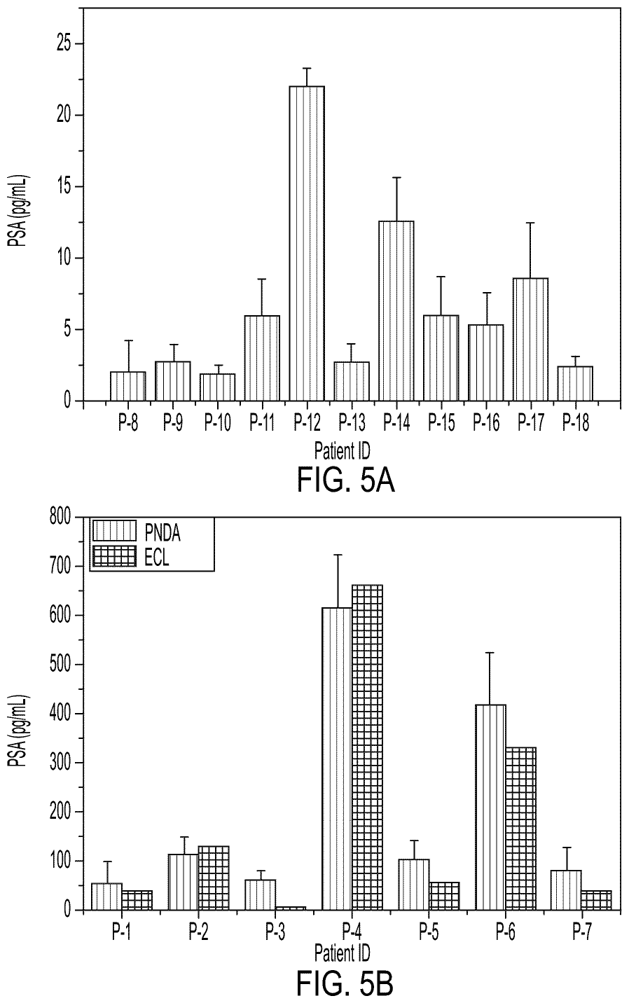

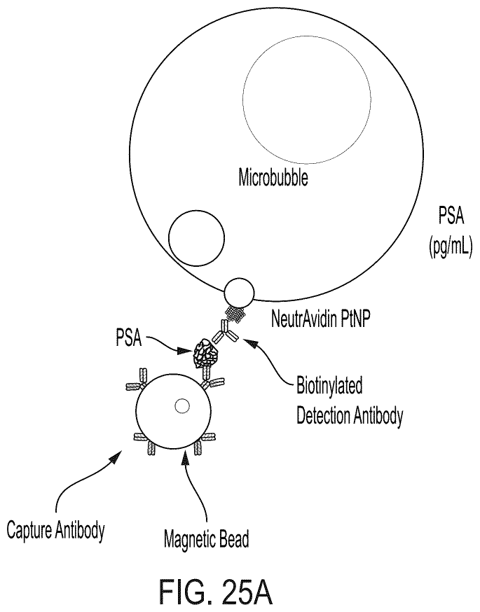

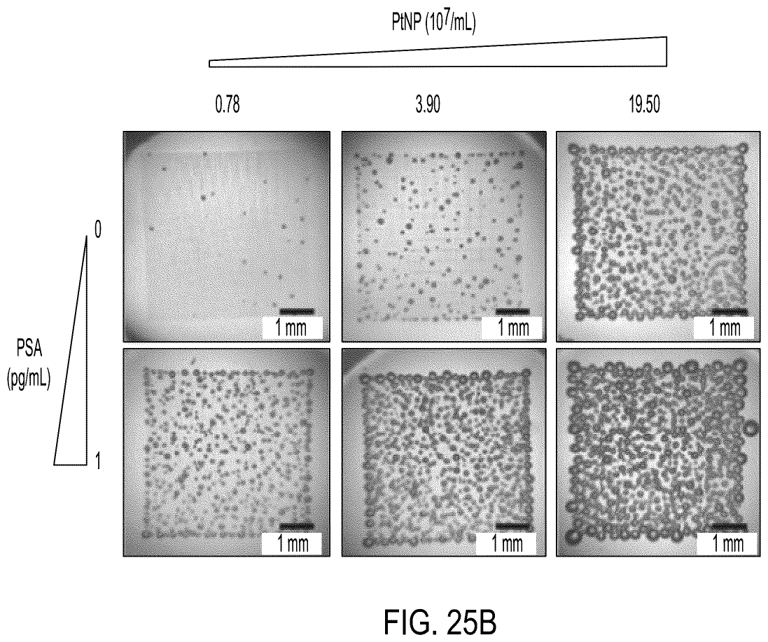

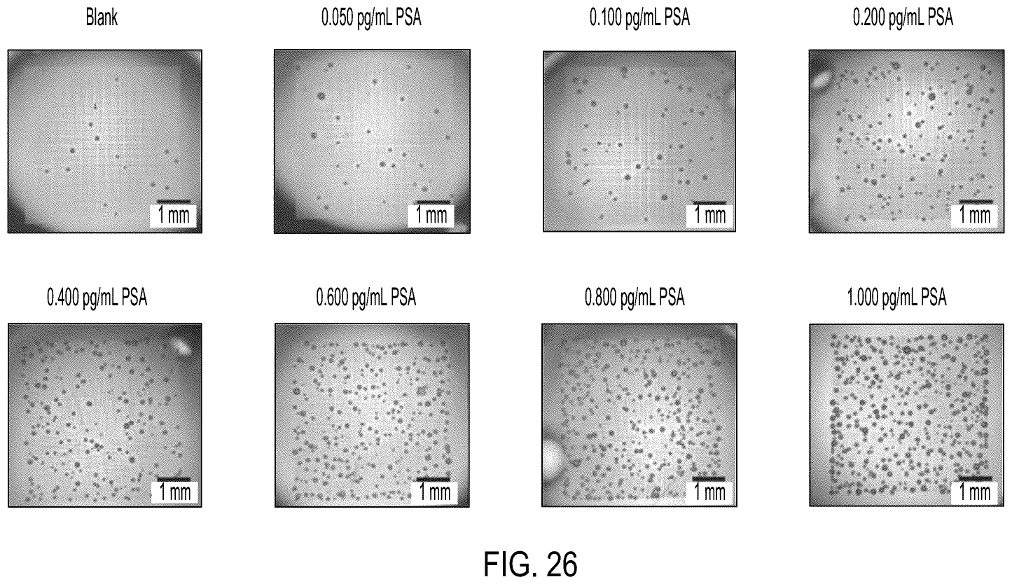

Here is provided an exemplary microbubbling assay to ultra-sensitively quantitate PSA for the post-prostatectomy surveillance of prostate cancer, in which the smartphone plays an integral role of data collection, analysis, and transmission. In this assay, paramagnetic microbeads were functionalized with monoclonal anti-PSA antibodies to capture the PSA molecules. As an example, biotinylated polyclonal antibodies were used to label the captured PSA molecules with NeutrAvidin functionalized PtNPs at the optimized concentration (FIGS. 25A-25B). As shown in FIGS. 5A and 5B and in FIG. 26, the number of microbubbles increased as the concentration of PSA increased, and reached plateau at around 500 microbubbles, at which time the bubble density became so high that adjacent microbubbles started to fuse, thus leading to a saturated signal. The dynamic range can be expanded by increasing the area or number of the microwell array on the chip.

Within the dynamic range (0.060-1 pg/mL), the number of microbubbles correlated linearly with the concentrations of PSA, with a limit of detection (LOD) of 2.1 fM (0.060 pg/mL) PSA. The LOD was calculated by extrapolating the PSA concentration at background plus 3 standard deviations of the background.

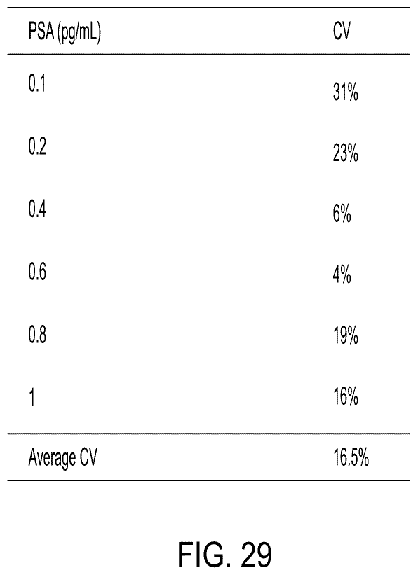

Compared with the current central clinical laboratory electrochemiluminescence (ECL) assay (Roche Elecsys Cobas Total PSA assay, lower reportable limit 0.01 ng/mL), microbubbling assay is 167 times more sensitive. At current stage, an average coefficient of variation (CV) of 16.5% has been achieved for the detection of PSA with microbubbling assay, as shown in FIG. 29. The CV of microbubbling assay can be further decreased by integrating the platform with automated microfluidic sample preparation, reaction mixing and washing.

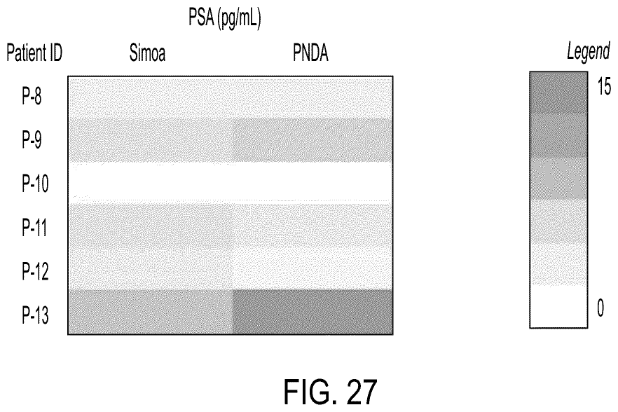

To validate the performance of the microbubbling assay in PSA quantitation, 13 deidentified prostate cancer patients' serum samples with various PSA concentrations were tested. As shown in FIG. 5C, the microbubbling results correlated well with the central clinical laboratory electrochemiluminescence (ECL) results. In the 6 samples with undetectable PSA with the ECL assay, the accuracy of the microbubbling results was validated against the Simoa research assay, as shown in FIG. 27.

To assess the versatility of the microbubbling assay, an assay for βhCG, an analyte for pregnancy was developed. High sensitivity βhCG detection in the clinical (e.g., POC) setting is useful to quickly rule-in or rule-out of early pregnancy, which is useful for pregnancy screening before diagnostic radiography procedures in the emergency department, and care planning in the home setting. However, the sensitivity and accuracy of most POC βhCG tests are not as good as their central laboratory counterparts, and many are insufficient to detect very early pregnancy.

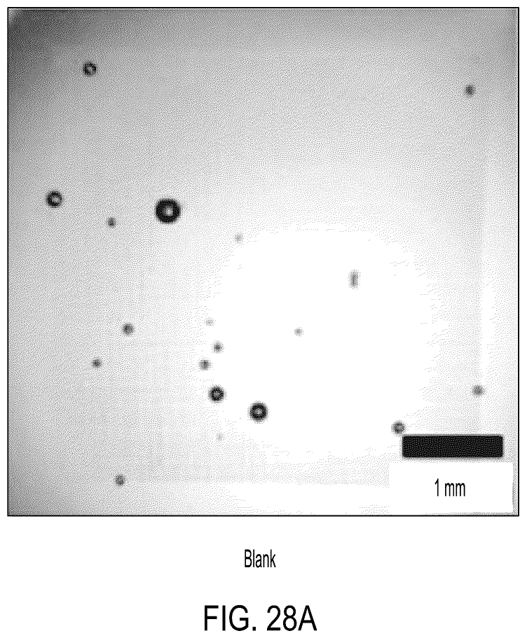

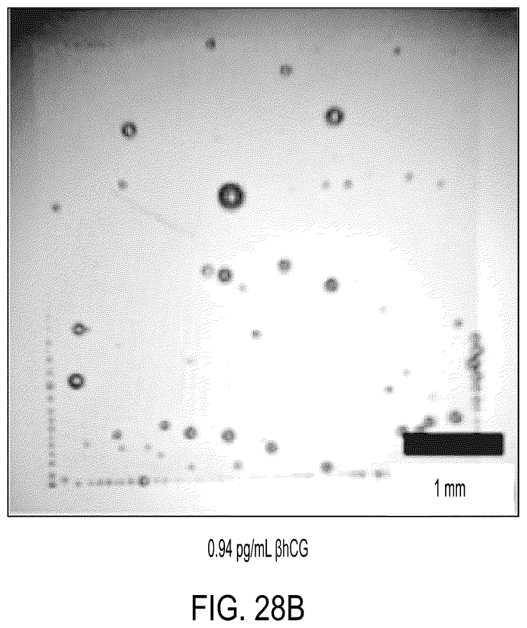

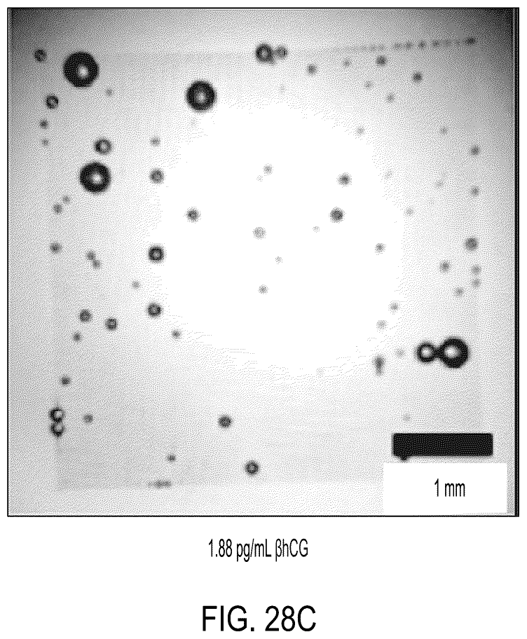

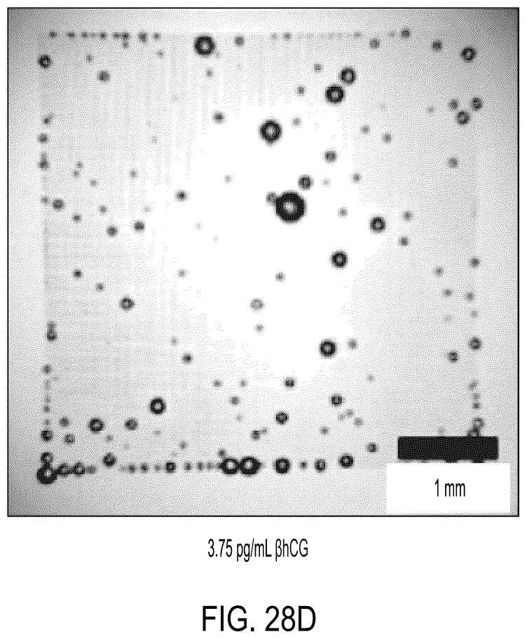

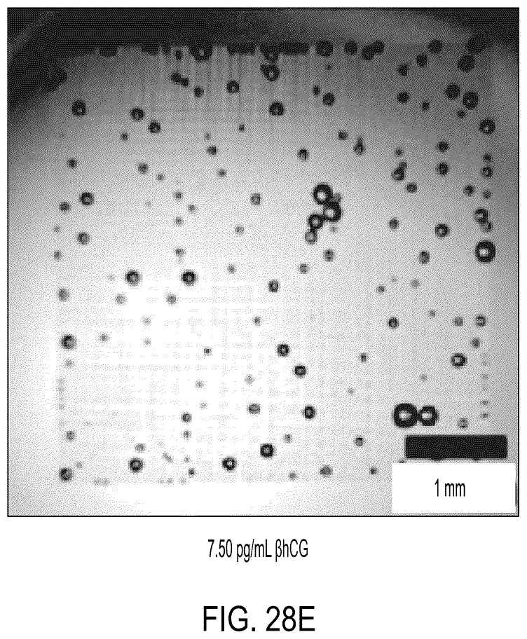

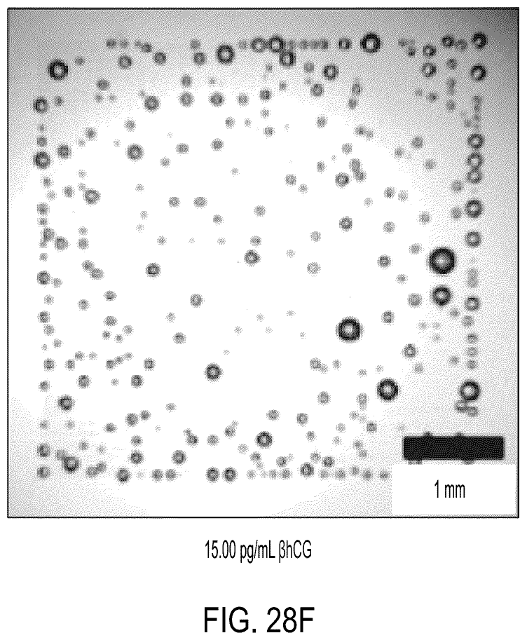

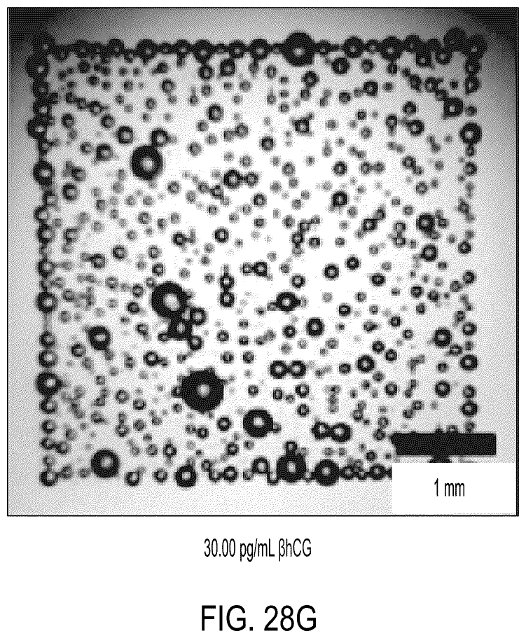

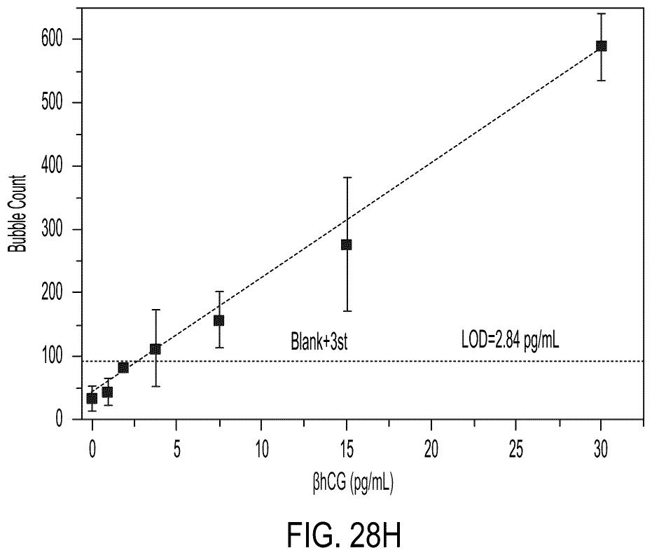

In one exemplary microbubbling assay, as shown in FIGS. 28A-28H, the number of microbubbles correlated linearly with the concentration of βhCG, with an LOD of 0.034 mIU/mL or 2.84 pg/mL or (background plus 3 standard deviations), with sensitivity significantly higher than current central laboratory (e.g., LOD: 0.5 mIU/mL or 42 pg/mL for Beckman Coulter chemiluminescence immunoassay (CLIA)) or POC assays (e.g., LOD: 5 mIU/mL or 0.4 ng/ml for Abbott i-STAT Total β-hCG Test).

The disclosed technology thus provides a novel, ultra-sensitive microbubbling digital assay readout method toward the clinical POC need of high sensitivity protein quantitation. It is demonstrated for the first time that immobilized-microbubbling can be used as a simple and fast digital assay signaling strategy to bridge the “invisible” nano-world to the “visible” micro-world. Compared with the ensemble volume or pressure analog signals of PtNP labels, the microbubbling assay uses “yes/no” digital signal that is less influenced by variations of environmental temperature and pressure, leading to lower background noises and higher sensitivity.

The microbubbling assay can be adapted to central laboratory instruments with high quality imaging capabilities for either research or diagnostic purposes. As described here, this technology can be used as a diagnostic, as microbubbles can be easily imaged with smart phone and mobile microscope.

Provided is an automated image analysis smartphone application via machine learning to make assay readout more user-friendly, robust and free of potential user bias. At current stage, multiple hands-on steps are still needed to carry out the incubation and washing steps in microbubbling assays. Further integration with automation systems, such as autonomous capillary microfluidic systems, disk-like microfluidic systems, and programmable electric wetting-based droplet mixing systems, allows the microbubbling assay to be further integrated by users. Once integrated, the ultra-sensitive microbubbling assay is a platform that has wide applicability beyond the two model protein analytes.

### Microbubbling Microchip for the Ultra-Sensitive Detection of Prostate-Specific Antigen (PSA)

Ultrasensitive PSA assessment in the post-prostatectomy surveillance of patients has utility as a means of risk stratification and counselling of patients on prognosis and treatment decisions. Early detection of recurrence offers the possibility of early salvage therapy given at a lower cancer burden and a wider time window for cure.

As mentioned elsewhere herein, postoperative PSA >0.073 ng/ml at day 30 significantly increased the risk of biochemical recurrence in the presence of positive surgical margins (PSM) after radical prostatectomy, demonstrating that ultrasensitive PSA can aid risk stratification in patients with PSM. Patients not likely to experience biochemical recurrence can be spared the toxicity of immediate adjuvant radiotherapy.

Other biochemical parameters for recurrence monitoring include PSA doubling time and PSA velocity, each of which requires repeated, sensitive and precise quantification of PSA. However, current central clinical laboratory assays require that patients repeatedly go to a phlebotomy station to have blood drawn and sent to a central laboratory for testing, with turn-around-time usually 1-2 days from blood draw to results. A quick-response system can thus save physicians and patients time, increase patient engagement, enable immediate discussion of the result and future management, minimize the stressful waiting period for test results, and possibly avoid administration of unnecessary additional treatment, thus significantly improving patient care efficiency.

Here is provided an illustrative microbubbling assay for the ultra-sensitive quantitation of PSA, in which the smartphone plays an integral role of data collection, analysis, and transmission. In this assay, paramagnetic microbeads were functionalized with monoclonal anti-PSA antibodies to capture the PSA molecules. Biotinylated polyclonal antibodies were used to label the captured PSA molecules with NeutrAvidin functionalized PtNP. As shown in FIG. 4E, the number of microbubbles on the microbubbling microchips correlated linearly with the concentrations of PSA, with a limit of detection (LOD) of 0.09 pg/mL PSA. The LOD was calculated by extrapolating the amount of PtNPs at background plus 3 standard deviations of the background. Compared with the current central clinical laboratory electrochemiluminescence (ECL) assay (Roche Elecsys Cobas Total PSA assay, lower reportable limit 0.01 ng/mL), microbubbling is 111 times more sensitive with the additional advantage of portable use.

### Validation of the Microbubbling Platform for the Ultra-Sensitive PSA Quantitation Using Patient Serum Samples

To validate the performance of microbubbling in PSA quantitation, 18 deidentified prostate cancer patients' serum samples with various PSA concentrations were tested. As shown in FIGS. 5A-5B, using microbubbling, PSA concentrations of 11 samples were successfully quantitated, which were undetectable using the central clinical laboratory assay (Roche Elecsys Cobas Total PSA assay, lower reportable limit 0.01 ng/ml). For the 7 samples whose PSA concentrations were high enough to be detected by the central clinical laboratory electrochemiluminescence (ECL) assay, microbubbling results were highly correlated with the ECL results.

### Automated Image Analysis Using Machine Learning

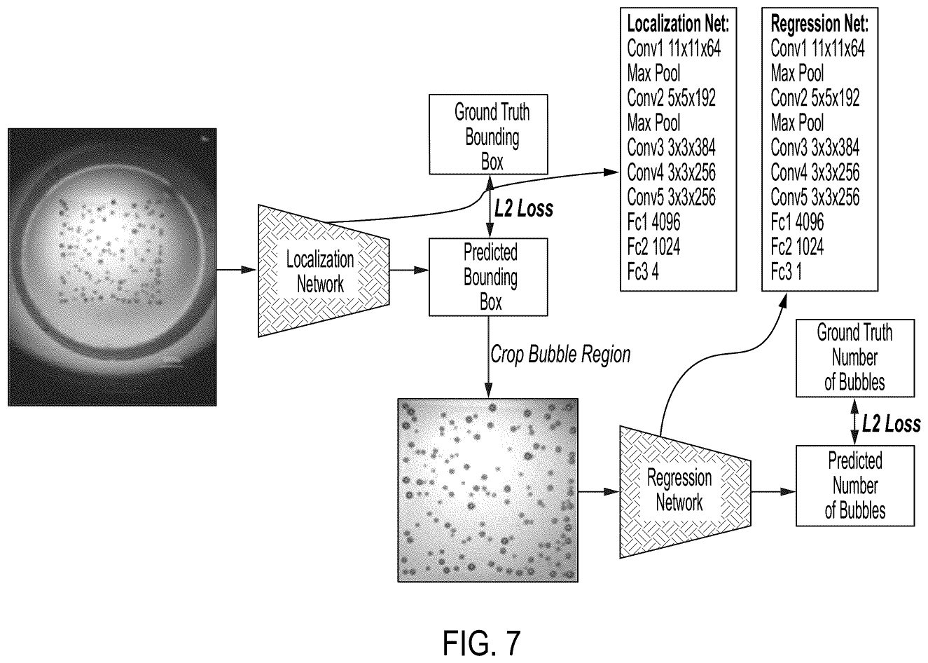

To make microbubbling more precise and user-friendly, and eliminate potential user bias in bubble counting, provided is an automated image analysis smartphone application via the localization-regression convolutional deep learning neural network. After training the algorithm with approximately 500 images (FIG. 7), the application successfully identified the boundaries of the microarray area and count the inside microbubbles in seconds.

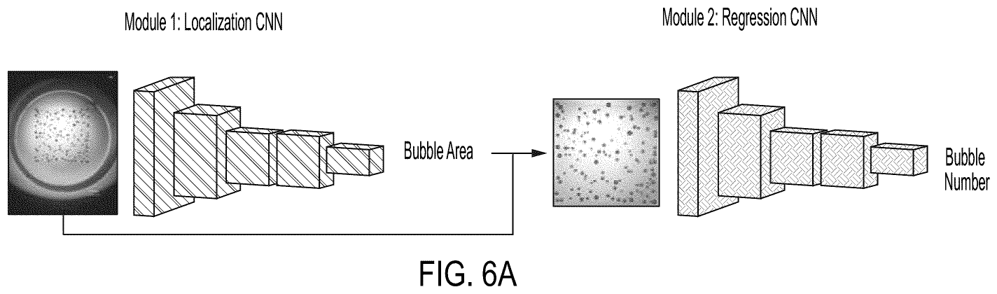

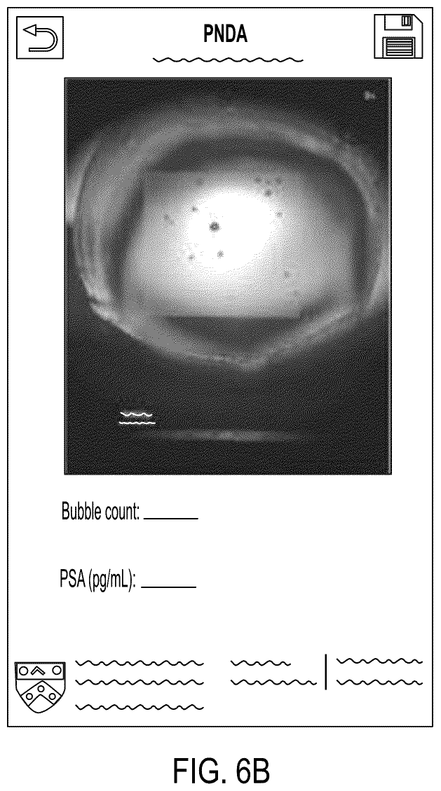

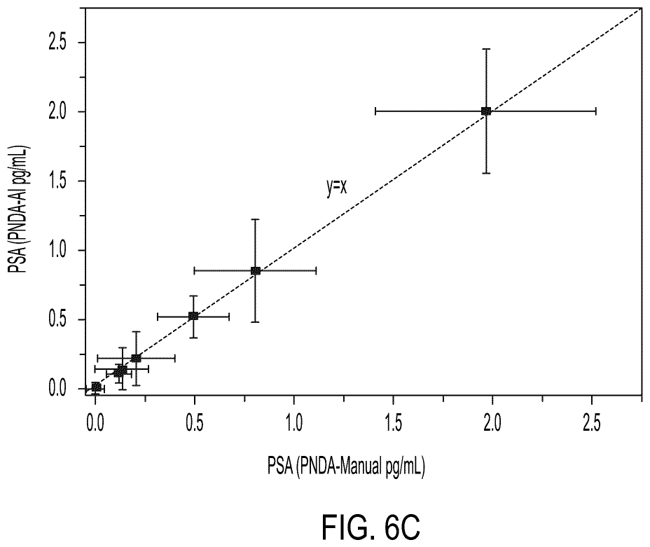

FIG. 6A provides a training approach via the deep learning network. Module 1 was built to learn how to localize the specific arears of the microwell arrays. Module 2 was used to learn how to count the number of microbubbles in the specific areas. FIG. 6B provides a user interface of the microbubbling smartphone application. FIG. 6C compares readouts via the artificial intelligence (AI) approach with ImageJ-assisted manual approach for PSA detections. Mean±standard deviation for microbubbling results; n=3. As shown in FIGS. 6A-6C, the readouts via the artificial intelligence (AI) application correlate well with the readouts via ImageJ assisted manual counting for PSA detections. The algorithm can be updated by increasing the amount of training data such as images taken by different untrained users to further reduce the rate of false positives and false negatives. With a smartphone as readout device, the microbubbling assay results can also be easily uploaded to a cloud-based server to be shared with care providers.

### Experimental Information—PSA

The following details are provided in connection with the illustrative results provided in this disclosure. The following details are illustrative only, and do not limit the scope of the present disclosure.

### Materials

Bovine serum albumin (BSA, A7906-50G), TWEEN® 20 (Molecular Biology Grade, P9416-100ML), and Nunc® MicroWell™ 96 well polystyrene plates (P7366-1CS), prostate specific antigen (PSA, human seminal fluid, 539832) were purchased from Sigma-Aldrich, Inc. (St. Louis, MO, USA). Sylgard™ 184 (24236-10) was purchased from Electron Microscopy Sciences (Hatfield, PA, USA). EZ-Link NHS-Biotin, (PI20217), ZebaTM spin desalting columns (89882), disposable standard biopsy punches (6 mm, 12-460-412), sodium azide (S2271100), Tris-HC buffer (1M, pH 8.0, 15568025), magnetic 96-well separator (A14179), Neodymium Disc Magnets (Grade: 35, S430471), hydrogen peroxide (30% in water, BP2633500), PierceTM premium grade Sulfo-NHS (PG82072), PierceTM premium grade EDC (PG82079), NeutrAvidin Protein (PI31000), sodium citrate (78-101-KG), Fisherbrand™ cover glasses (squares No. 1.5 18 mm, 12541A) were purchased from Thermo Fisher Scientific, Inc. (Rockford, IL, USA). LodeStars® High Bind Carboxyl magnetic beads (trial pack) were purchased from Agilent Technologies, Inc. (Santa Clara, CA, USA). Phosphate-buffered saline (PBS) tablets (T9181), pH 7.4, magnetic stand (631964) were purchased from Clontech Laboratories, Inc. (Mountain View, CA, USA). Mouse monoclonal anti-Prostate Specific Antigen (PSA) antibody (ABPSA-0405) was purchased from Arista Biologicals, Inc. (Allentown, PA, USA). Human Kallikrein 3/PSA biotinylated antibody (polyclonal goat, BAF1344) was purchased from R&D Systems, Inc. (Minneapolis, MN, USA). MES Buffer (50 mM, pH 6.0, 21420006-1) was purchased from Spectrum Chemical Manufacturing Corp. (New Brunswick, NJ, USA). Platinum Nanoparticles (140 nm, tannic acid surface) were purchased from Nanocomposix, Inc. (San Diego, CA, USA). KMPR Applications® 1050 photoresist, SU-8 developer were purchased from MicroChem Corp. (Westborough, MA, USA). Silicon wafers (452, 100 mm, 500 um) were purchased from Aidmics Biotechnology Co., LTD. (UniversityWafer) (Boston, MA, USA). The uHandy Mobilephone Microscope (Duet set) was purchased from Aidmics Biotechnology Co. (Taipei, Taiwan, China).

### Deidentified Human Serum Samples

Deidentified (anonymized) serum samples with various PSA concentrations were obtained from ARUP Laboratories (Salt Lake City, Utah, USA). PSA concentrations were measured using Roche Elecsys Cobas Total PSA assay (lower reportable limit 0.01 ng/mL). Leftover serum after clinical testing was frozen until tested using the microbubbling assay.

### Design and Fabrication of Microbubbling Microchips

The microbubbling microchip included three layers: commercial cover glass (18 mm×18 mm×150 μm) as the bottom supporting layer; PDMS sheet (˜1 cm×1 cm) that contains an array (100×100) of micro wells (14 um×14 μm×7 μm) and is surface coated with parylene (3 μm) as the middle layer; and a PDMS top layer containing a round chamber (Ø6 mm, 5 mm) for sample loading.

The mold of the middle PDMS layer was made of KMPR® 1050 photoresist on Si wafer through conventional photolithography. The new 100 mm Si wafer is first prebaked at 200° C. for 10 min. Then about 5 mL of KMPR® 1050 photoresist was poured on the surface of the wafer and spin using an SU-8/PDMS Resist Spinner (SINGH center for Nanotechnology, PA, USA) at 1000 rpm for 30 s to form a 10 μm layer. The wafer was then baked at 100° C. for 6 min. The photoresist was then exposed under UV light with exposure energy of 336 mJ/cm2 using an AMB 3000 HR Mask Aligner Spinner (SINGH center for Nanotechnology, PA, USA). After exposure, the wafer was baked at 100° C. for 2 min. The photoresist on the wafer was further treated with SU-8 developer until the clear patterns appeared, followed by rinsing with acetone and isopropyl alcohol. The PDMS base and curing agent were mixed thoroughly at 10:1 ratio and poured over the mold. Following vacuum degas for 30 min, the PDMS mixture covered mold was baked at 75° C. overnight.

To make the PDMS top layer, PDMS base and curing agent were mixed thoroughly at 10:1 ratio and poured in a petri dish with a flat bottom. Following vacuum degas for 30 min, the PDMS mixture was baked at 75° C. overnight. Then the PDMS was peeled out of the petri dish and cut into ˜1 cm×1 cm squares. Then a round whole with a diameter of 6 mm was punched at the center of each square using biopsy punches.

The three layers of the microbubbling microchip were assembled together with the microwell array on the middle layer facing upward and centered at the chamber in the top layer. Then the assembled microbubbling microchips were treated with a parylene coater (LABCOTER®2, Specialty Coating Systems, Inc. Indianapolis, IN, USA) to form a 3 μm parylene layer on the surface.

### Functionalization of Platinum Nanoparticles

For the preparation of NeutrAvidin-conjugated PtNPs, 20 μL of 5 mg/mL NeutrAvidin were mixed with 1 mL of 0.05 mg/mL 140 nm PtNPs in citrate buffer, pH 7.2, and continuously mixed using a rotator (20 rpm) at 4° C. overnight. Then to block the PtNP surface, 100 μL of 10% BSA in citrate buffer, pH 7.2 was added and mixed with PtNPs using a rotator (20 rpm) at 4° C. overnight. Unconjugated Neutravidin was removed by centrifugation at 2400 g 6 times for 8 min each. Finally, the NeutrAvidin-conjugated PtNPs were suspended in 100 μL of PBS, pH 7.4, containing 1% BSA.

### Functionalization of Superparamagnetic Microbeads

LodeStars® High Bind 2.7-μm diameter carboxyl-terminated superparamagnetic beads were functionalized with a monoclonal antibody to prostate specific antigen (PSA) using EDC coupling following the manufacturer's instructions. Briefly, 50 μL of ˜2.9×109/mL beads were first rinsed and twice with 100 μL of 0.01 M sodium hydroxide to activate the carboxy groups on the beads. Then the beads were rinsed 3 times with 100 μL deionized water following 3 times rinsing with MES buffer, pH 6.0. Then the beads were further reacted with 100 μL solution containing 50 mg/mL of Sulfo-NHS and 50 mg/ml EDC in MES buffer, pH 6.0 on a roller (20 rpm) at 23° C. for 25 min. After 2 times quick rinses with 100 μL MES buffer, pH 5.0, the beads were reacted with 100 μL of 3 mg/mL monoclonal antibody in MES buffer pH 5.0 at 4° C. overnight. To quench the uncoupled NHS group on the surface, the beads were further reacted with 100 μL of 100 mM Tris-HCl, pH 7.4 at 4° C. for 2 h. Finally, the antibody functionalized beads were rinsed 3 times with 600 μL of PBS buffer pH 7.4 containing 1% BSA, and then resuspended in 1 mL PBS buffer pH 7.4 containing 1% BSA and 0.02% sodium azide for storage.

To functionalize the superparamagnetic beads with biotinylated BSA, the beads were first functionalized with BSA using a similar protocol as above, except the 3 mg/mL antibody solution was replaced with 10 mg/mL BSA solution. The BSA functionalized beads were further reacted with 5 mM NHS-Biotin in PBS buffer pH 7.4 on a roller (20 rpm) at 23° C. for 1 h. Finally, the biotinylated BSA functionalized beads were rinsed 3 times with 600 μL of PBS buffer pH 7.4 containing 1% BSA, and then resuspended in 1 mL PBS buffer pH 7.4 containing 1% BSA and 0.02% sodium azide for storage.

Quantitation of βhCG with Microbubbling Microchips

Test solutions (100 μL) of different concentrations of βhCG were incubated with suspensions of 500,000 anti-βhCG monoclonal antibody functionalized magnetic beads, on a roller (12 rpm) at 23° C. for 2 h. The beads were then separated using a strong magnets and washed 3 times with 300 μL of PBS buffer pH 7.4 containing 1% BSA and 0.01% TWEEN® 20, and then resuspended in 100 μL of 150 ng/ml biotinylated anti-βhCG monoclonal antibody in PBS containing 1% BSA, on a roller (12 rpm) at 23° C. for 1 h. The beads were then separated using strong magnets and washed 3 times with 300 μL of PBS buffer pH 7.4 containing 1% BSA and 0.01% TWEEN® 20, and then resuspended in 100 μL of 1 μg/mL NeutrAvidin functionalized PtNP in PBS containing 1% BSA, on a roller (12 rpm) at 23° C. for 30 min. The beads were then separated using strong magnets and then resuspended in 100 μL of 30% H2O2. The magnetic beads slurries were then applied into the chambers of the microbubbling microchips. Then the microbubbling microchips were placed on neodymium disc magnets for 1 min to pull down the beads to the bottom of the microchips. Finally, within 10 min, different number of microbubbles with diameter ranging from 20 μm to 60 μm were observed in the microwell arrays with either microscope or cell phone.

### Quantitation of NeutrAvidin Functionalized PtNP with Microbubbling Microchip

Test solutions (100 μL) of different amount of NeutrAvidin functionalized PtNP with 1% BSA in PBS pH 7.4 were incubated with suspensions of 200,000 biotinylated BSA functionalized magnetic beads, on a roller (12 rpm) at 23° C. for 30 min. The beads were then separated using a magnetic separator and then resuspended in 100 μL of 30% H2O2, 0.05% TWEEN® 20. The magnetic beads slurries were then applied into the chambers of the microbubbling microchips. Then the microbubbling microchips were placed on neodymium disc magnets for 1 min to pull down the beads to the bottom of the microchips. Finally, within 10 min, different number of microbubbles were observed in the microwell arrays with either laboratory microscope or mobile microscope (9×) and smartphone.

### Quantitation of PSA with Microbubbling Microchips

Test solutions (100 μL) of different concentrations of PSA were incubated with suspensions of 200,000 anti-PSA monoclonal antibody functionalized magnetic beads, on a roller (12 rpm) at 23° C. for 2 h. The beads were then separated using a magnetic separator and washed 3 times with 300 μL of PBS buffer pH 7.4 containing 1% BSA and 0.01% TWEEN® 20, and then resuspended in 100 μL of 150 ng/mL biotinylated anti-PSA polyclonal antibody in PBS containing 1% BSA. The mixture was then place on a roller (12 rpm) at 23° C. for 1 h. The beads were then separated using a magnetic separator and washed 3 times with 300 μL of PBS buffer pH 7.4 containing 1% BSA and 0.01% TWEEN® 20, and then resuspended in 100 μL of 1 μg/mL NeutrAvidin functionalized PtNP in PBS containing 1% BSA. The mixture was then place on a roller (12 rpm) at 23° C. for 30 min. The beads were then separated using a magnetic separator and then resuspended in 100 μL of 30% H2O2, 0.05% TWEEN® 20. The magnetic beads slurries were then applied into the chambers of the microbubbling microchips. Then the microbubbling microchips were placed on neodymium disc magnets for 1 min to pull down the beads to the bottom of the microchips. Finally, within 10 min, different number of microbubbles were observed in the microwell arrays with either laboratory microscope or mobile microscope (9×) and smartphone.

### Imaging and Analysis of Microbubbling Assay Output

The microbubbles on the microbubbling microchips were either imaged with conventional laboratory microscope or iphone 6 Plus with the uHandy mobilephone microscope (9×, 5 mm focusing length, Aidmics Biotechnology Co. Taipei, Taiwan, China), followed by analysis with NIH ImageJ 1.43U (Dr. Wayne Rashand, National Institutes of Health, USA).

When there were no microbubbles adjacent to each other, the images were first thresholded black and white from value 0 to 145, and then analyzed with the “Analyze Particle” function to obtain the number of microbubbles. For the images with microbubbles adjacent to each other, they were analyzed with the “Cell Counter” plugin to obtain the number of microbubbles manually.

### Automated Image Analysis Smartphone Application Development Using Machine Learning

A Localization-Regression Network was generated for counting the number of microbubbles in the cellphone images, which first identifies and crops the microwell array area, and then counts the number of microbubbles inside. The idea of the localization network is to filter out the irrelevant regions in the images and enable the following counting regression network more efficiently and accurately. The first five convolutional layers of the localization network and the regression network can be compared to the AlexNet 1 architecture.

All convolutional layers are followed by ReLU activation function and batch normalization, and two dropout layers with 0.5 dropout probability were used in the first and second fully connected layers in both networks. The localization network outputs four values representing two corners of the squared microarray area, and the regression outputs one value representing the final microbubble count. To train this network, 493 images were taken by an iphone 6 Plus at various imaging conditions (FIGS. 18A-18B) and labeled each image with a bounding box and the number of microbubbles. During the training process, the regression network was trained for 3,000 epochs until it converged, and the network was trained for another 3,000 epochs with batch size of 64. The L2 loss was used to penalize both the predicted bounding box and the bubble regression and used the Adam optimizer with learning rate of 0.0005, beta 1 of 0.9, and beta 2 of 0.999 to optimize the network.

### Illustrative Disclosure—LFA Ruler

Conventional lateral flow assays (LFA) s provide qualitative or semi-quantitative results, and require dedicated instruments for quantitative detection. Provided here is what is termed a “LFA Ruler” for quantitative and sensitive readout of LFA results, using a simple, inexpensive microfluidic chip.

Platinum (or other) nanoparticles are used as signal amplification reporter, which catalyze the generation of oxygen (or other product) to push ink advancement in the microfluidic channel. The concentration of target is linearly correlated with the ink advancement distance. The entire assay can be completed within 30 minutes without external instrument and complicated operations. Here are demonstrated quantitative prostate specific antigen testing using LFA ruler, with a limit of detection of 0.54 ng/mL, linearity between 0-12 ng/mL, and high correlation with clinical gold standard assay. The LFA ruler achieves low cost, instrument-free, quantitative, sensitive and rapid detection, which can be extended to quantify other disease analytes.

In conventional LFAs, qualitative or semi-quantitative results are generated by visual inspection in less than 30 min. Direct visualization of a colorimetric LFA readout is very useful for clinicians to make an immediate medical decision. However, there can exist subjective judgment variation in visual interpretation with the naked eye among end-users, caused by the differences of illumination setting and personal visual ability and other psychological factors. Thus, it could lead to uncertain readouts, especially when the colorimetric signal is close to threshold. Further, the sensitivity and quantification ability of LFA are intrinsically limited by the colorimetric signal readout. The need for additional readers also increases overall testing costs.

Quantitative LFAs utilizing fluorescence, magnetic or Raman reporters instead of colorimetric labels have also been developed. Although these strategies contribute to improvement in the sensitivity of LFAs and expand their applications, they all require additional dedicated and sophisticated instruments for readout and experienced operators for quantitative analysis. These factors render the above strategies unsuitable for use in resource-limited settings.

Provided here are simple, inexpensive microfluidic chips for LFA quantitation and sensitive detection with distance-based readout, which can be termed “LFA Ruler”. After the conventional operation of PtNP-based LFA, the test zone is further cut and added to the reaction chamber in LFA ruler. PtNP-catalyzed oxygen generation in H₂O₂ solution pushes colored ink to advance in the microfluidic channel. The ink advancement distance, read directly with the naked eye, is linearly correlated with the concentration of target.

One can apply the LFA ruler in, as but some examples, quantification of prostate specific antigen (PSA) in clinical serum samples, and compare LFA results with commercial electro-chemiluminescence immunoassay (ECLIA). PSA is a protein produced mostly by cells of the prostate gland, and is used clinically as a prostate cancer screening biomarker. Globally, prostate cancer is the second most common type of cancers and the fifth leading cause of cancer-related deaths in men. Many studies suggested that prostate cancer mortality can be decreased by early screening. A serum PSA concentration below 4 ng/mL in screening indicates low probability of prostate cancer; concentration above 10 ng/ml indicates possible presence of prostate cancer; concentration between 4 and 10 ng/ml is within the so-called grey zone and indicates that further definitive testing may be warranted. The LFA ruler enables low cost, instrument-free, quantitative, and sensitive readout of PtNP-based PSA LFA strip, allowing clinical decision making with relation to the above thresholds, which is especially suitable for use in resource-limited areas. Moreover, as a versatile platform, the LFA can be used in quantification of other disease biomarkers besides PSA.

### Materials and Chemicals

Glass slides (75×50×1 mm³ and 75×25×1 mm³) were purchased from Corning, Inc. (Corning, NY, USA). Silicon wafers (100 mm) were purchased from University Wafer (Boston, MA, USA). KMPR-1050 photoresist and SU-8 developer were purchased from MicroChem Corp. (Newton, MA, USA). Polydimethylsiloxane (PDMS) elastomer kits (Sylgard™ 184) were purchased from Electron Microscopy Sciences (Hatfield, PA, USA). Platinum Nanoparticles (70 nm) were purchased from Nanocomposix, Inc. (San Diego, CA, USA). Bovine serum albumin (BSA, A7906-50G), Tween-20 (Molecular Biology Grade, P9416-100ML), 1H, 1H,2H,2H-Perfluorooctyltrichlorosilane (97%), and prostate specific antigen (PSA, human seminal fluid, 539832) were purchased from Sigma-Aldrich, Inc. (St. Louis, MO, USA). EZ-Link NHS-Biotin, (PI20217), Zeba™ spin desalting columns (89882), HABA (4′-hydroxyazobenzene-2-carboxylic acid, 28010), disposable standard biopsy punches (6 mm, 12-460-412), hydrogen peroxide (30% in water, BP2633500), NeutrAvidin Protein (PI31000), sodium citrate (78-101-KG), red ink and sealing tape for 96-well plates were purchased from Thermo Fisher Scientific, Inc. (Rockford, IL, USA). Phosphate-buffered saline (PBS) tablets (T9181), pH 7.4, were purchased from Clontech Laboratories, Inc. (Mountain View, CA, USA). Mouse monoclonal anti-PSA antibodies (ABPSA-0405 and ABPSA-0406) were purchased from Arista Biologicals, Inc. (Allentown, PA, USA). Goat anti-mouse IgG (ABGAM-0500) was purchased from Arista Biologicals, Inc. (Allentown, PA, USA). Polyethylene glycol (PEG) 3350 was purchased from GoldBio Inc. (St. Louis, MO, USA). Scotch tape was purchased from 3M (Maplewood, MN, USA). The glass fiber (G041) was obtained from EMD Millipore Corporation (Billerica, MA, USA). The Fusion 5 membrane, nitrocellulose membrane (FF 80HP) and absorbent paper (GB003) were purchased from GE Healthcare Life Sciences (Pittsburgh, PA, USA). The backing card was purchased from DCN Dx (Carlsbad, CA, USA).

### Design and Fabrication

The microfluidic chip of a LFA ruler was composed of one layer of PDMS bonded to a glass slide, fabricated with conventional soft lithography techniques.

First, a clean 4-inch silicon wafer was baked at 200° C. for 10 min to promote dehydration. Then, KMPR-1050 photoresist was spin-coated on the wafer (3000 rpm for 30 s) to create a 50-μm photoresist layer. After soft baking at 100° C. for 15 min, the chip patterns on a Chrome photomask were transferred onto the photoresist via UV exposure using an exposure dose of 960 mJ/cm2 (AMB 3000 HR Mask Aligner, 365 nm). The microchannel in LFA ruler was 150-μm in width and 50-μm in height. The wafer was placed onto a hot plate (100° C.) for 5 min to perform post-baking, following by developing in bath of SU-8 developer with constant agitation and rinsing in acetone and isopropyl alcohol (IPA) to wash away the unexposed photoresist. The mold was dried using nitrogen gun and hard-baked at 150° C. for 30 min. Then, the mold was silanized with 1H, 1H,2H,2H-perfluorooctyltrichlorosilane in a desiccator overnight at room temperature to prevent undesired bonding between PDMS and the mold.

Second, PDMS base and PDMS curing agent at 10:1 ratio by weight were vigorously mixed and poured over the mold in a circular aluminum dish. After degassing the PDMS mixture in a vacuum chamber for 30 min, the dish was placed on a hotplate at 100° C. for 45 min. The PDMS replica was peeled from the mold and the inlets and outlets of microchannel were punched using biopsy punches.

Third, a clean glass slide and PDMS replica were carefully bonded together after oxygen plasma treatment for 40 s (Anatech SCE-106 Barrel Asher, 50 sccm, 50W). The hydrophobic treatment reagent (1H, 1H,2H,2H-perfluorooctyltrichlorosilane in IPA, 1% v/v) was injected into the microchannel through the outlet after heating for 10 s at 100° C. Then, the chip was placed on a hotplate at 100° C. for 1 hour to achieve hydrophobic treatment of the inside of the microchannel and irreversible bonding between PDMS and glass.

### Preparation and Conjugation of Platinum Nanoparticles

For preparation of NeutrAvidin-conjugated PtNPs, 20 μL of 5 mg/ml NeutrAvidin were mixed with 1 mL of 0.05 mg/mL 70 nm PtNPs in citrate buffer, pH 7.2, and continuously mixed using a rotator (20 rpm) at 4° C. overnight. Then BSA was added to a final concentration of 1% and mixed on a rotator (20 rpm) at 4° C. overnight to block the PtNPs surface. Unconjugated NeutrAvidin was removed by centrifugation at 3000 g 6 times for 8 min each. Finally, the NeutrAvidin-conjugated PtNPs were suspended in 100 μL of PBS, pH 7.4, containing 1% BSA.

Biotinylation of monoclonal anti-PSA antibody (ABPSA-0406) with Pierce premium-grade NHS-Biotin was performed according to the manufacturer's protocol. Briefly, the protein was mixed with NHS-Biotin (mole ratio, 1:20), and the reaction was allowed to occur at room temperature for 30 min. The uncoupled NHS-Biotin was removed with a Zeba™ desalting column (40 kDa molecular weight) according to the manufacturer's protocol. The biotinylated antibodies were stored with 1% BSA in PBS pH 7.4 at 4° C.

For the preparation of antibody-platinum nanoparticle (Ab-PtNP) conjugates, 25 μL biotinylated antibody were mixed with 1 mL of NeutrAvidin-conjugated PtNPs in PBS buffer, pH 6.5, and continuously mixed using a rotator (20 rpm) at 4° C. overnight. BSA was added to a final concentration of 1% to block the PtNPs surface, and unconjugated antibody was removed via centrifugation. Finally, the antibody-conjugated PtNPs were suspended in 500 μL of PBS, pH 7.4, containing 1% BSA, and stored at 4° C.

### Lateral Flow Strips Preparation

An illustrative (but non-limiting) test strip was constructed with four main elements: the sample pad, the conjugate pad, the nitrocellulose membrane, and the absorbent pad. The four parts were pasted on a plastic backing one by one, with ends overlapping 2 mm. Then, strips with widths of 4 mm each were produced using a paper cutting machine. A Fusion 5 membrane was used as the sample pad because of its low non-specific binding.

The glass fiber membrane was used as the conjugate pad, pretreated with 10% sucrose to improve the stability of Ab-PtNP conjugates. The conjugates were rinsed into the glass fiber, and air dried at room temperature.

Monoclonal anti-PSA antibody (ABPSA-0405) and goat anti-mouse IgG were separately diluted in phosphate buffer (PBS, 0.01 M, pH 7.4). The diluted antibodies were applied onto the nitrocellulose membrane to generate the test zone and control zone, respectively. The strips were dried at 37° C. and relative humidity of 25 to 30% overnight and stored at room temperature in a sealed package with silica gel.

### Quantifying Lateral Flow Assay Results

Fifty microliters of LFA buffer (0.01 M PBS, pH 7.4; 0.1% Tween-20; 0.2% BSA; 0.1% PEG-3350) containing different concentrations of analytes was loaded into a 2 mL Eppendorf tube lid. Then the sample pad of the LFA strip were inserted into the lid and reacted for 15 min at room temperature. Alternatively, buffer containing the analytes can be directly applied onto the sample pad. After that, the test zone (4×4 mm²) was cut and transferred into the reaction chamber of LFA ruler, and 3 μL of red ink was loaded into the ink chamber. Finally, 35 μL H₂O₂ (30%) was added into the reaction chamber. To seal the device, a piece of sealing tape (15×20 mgm²) was gently pasted on top of the reaction chamber and another piece of Scotch tape was gently pasted on top of the ink chamber and the balance reservoir. After incubation for 12 min at room temperature, the ink advancement distances were read directly with the naked eye. After 12 minutes of incubation, photos showing oxygen bubbles were captured using a cellphone with a uHandy Mobilephone Microscope (Aidmics Biotechnology Co., Taipei, Taiwan, China).

### Clinical Serum Sample Collection and Analysis

All deidentified human serum samples were obtained from ARUP Laboratories (Salt Lake City, UT, USA). All serum samples were first analyzed using the commercial ECLIA method (Roche Elecsys Cobas Total PSA assay), then frozen till tested using the LFA ruler. The study is approved by the institutional IRB committee. For analysis using the LFA ruler, the samples were diluted with LFA buffer (0.01 M PBS, pH 7.4; 0.1% Tween-20; 0.2% BSA; 0.1% PEG-3350) and then analyzed as described above, in triplicate. The results are shown as mean±standard error.

### Working Principles

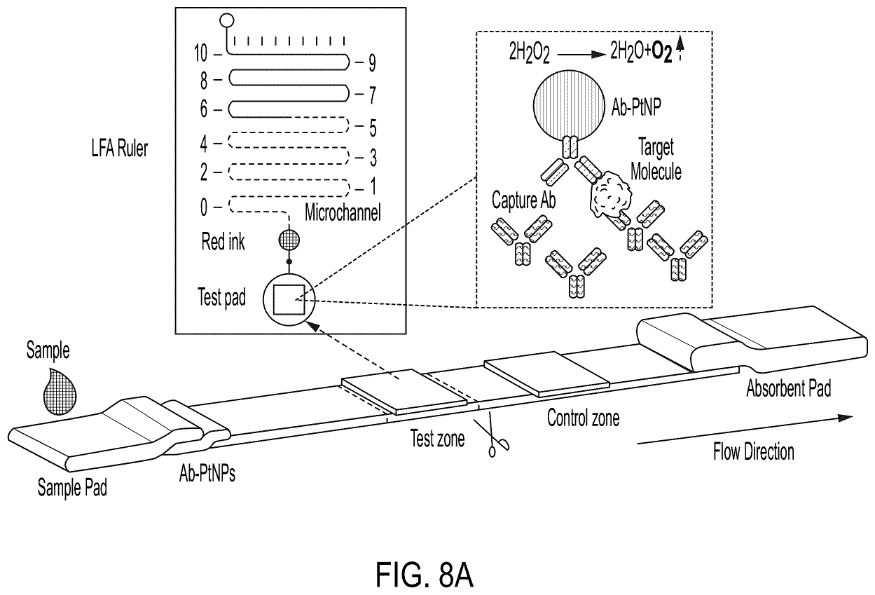

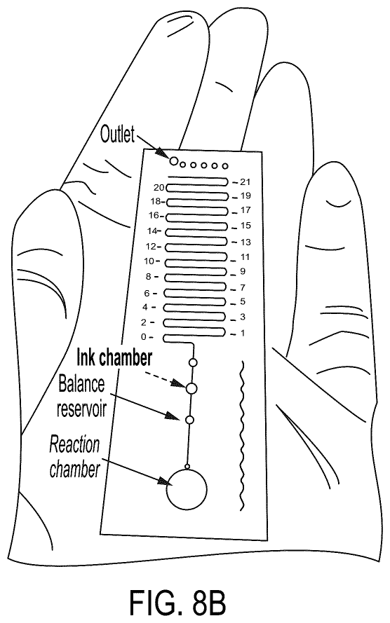

Without being bound to any particular theory, the working principle of the LFA ruler is shown in FIG. 8a. The LFA strip is composed of a sample pad, a conjugate pad, a nitrocellulose membrane, and an absorbent pad, which are successively assembled on a plastic backing card. Sample solution is applied on the sample pad and flows toward the absorbent pad driven by capillary force. Target molecule in the sample solution binds to the pre-immobilized detection Ab-PtNP conjugates when flowing through the Ab-PtNP conjugation pad. The PtNPs labeled target molecule are captured by the target-specific capture antibody (Ab) in the test zone and the excess conjugates migrate further and bind to the anti-mouse capture Ab in the control zone. The entire test zone pad is further cut and added to the reaction chamber in the microfluidic chip. The PtNPs captured in the pad can catalyze the oxidation of H₂O₂ into water and oxygen. The generated oxygen is sealed in the chip and pushes the ink forward in the microchannel. The ink advancement distance of test zone within a specified time period is read directly with the naked eye, which is proportional to the amount of target molecules in the sample. Furthermore, the control zone pad can be also tested like the test zone pad, which functions as the internal quality control of the LFA strip. Unlike previous LFA quantitative readout methods, the LFA ruler achieves the direct visualization of assay results without the need for external instruments.

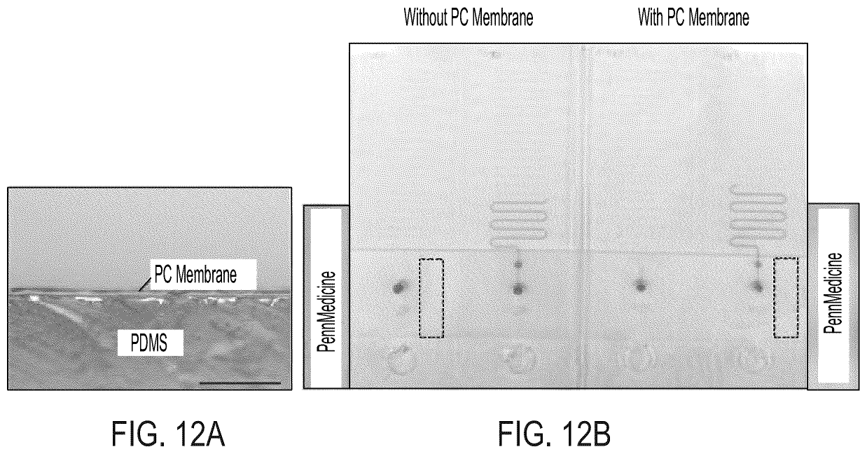

The LFA ruler is based on a PDMS-glass hybrid microfluidic chip, which is low cost and easy to fabricate using conventional soft lithography techniques. To test if PDMS needs to be treated to enhance gas impermeability, a 3-μm-thick layer of low-permeability Parylene C (PC) membrane was deposited on the surface of the LFA ruler (LABCOTER®2, Specialty Coating Systems Inc., IN, USA), including the entire interior of the chambers (FIG. 12A). Under the same experimental conditions, the distance of ink advancement in the device with PC membrane was only a little longer than that in the device without PC membrane (FIG. 12B). This can be explained by the fact that the LFA ruler is an open-ended device, so the effect of gas permeability of PDMS is not significant. All subsequent experiments were conducted on the devices without PC membrane.

Furthermore, the elasticity of PDMS might change the internal pressure when the tape is applied to the surface to seal the chambers. To address this issue, a balance reservoir can be added between the reaction chamber and the ink chamber, and the sealing process is changed to two-step method. When the reaction chamber is sealed, the balance reservoir can keep the internal pressure the same as the atmospheric pressure, eliminating interference caused by PDMS deformation. Then, the ink chamber and the balance reservoir are sealed successively by adding another tape. FIG. 8b shows a photograph of the LFA ruler, including the microfluidic channel, an ink chamber, a balance reservoir, a reaction chamber, and an outlet.

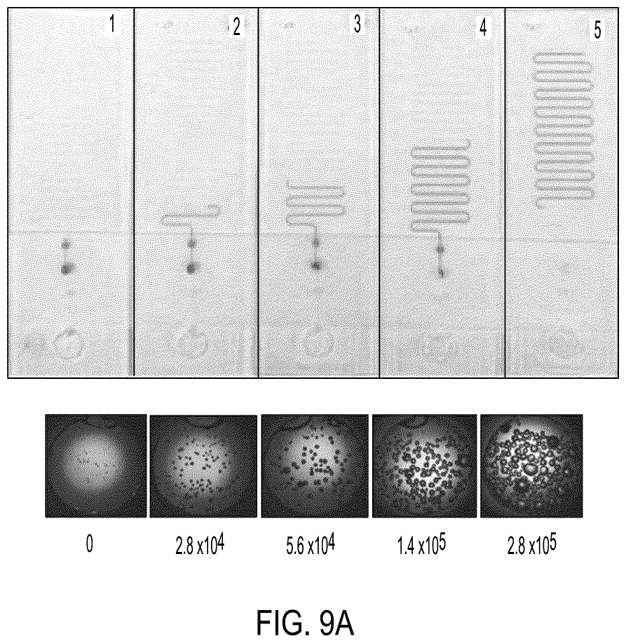

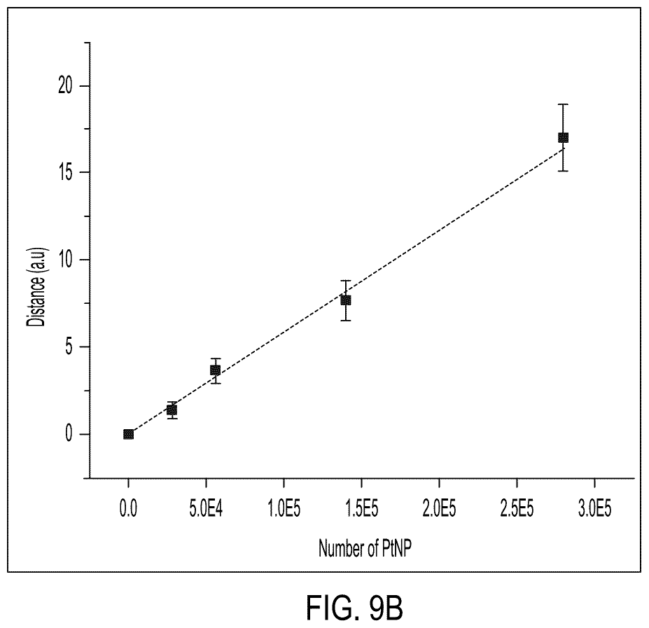

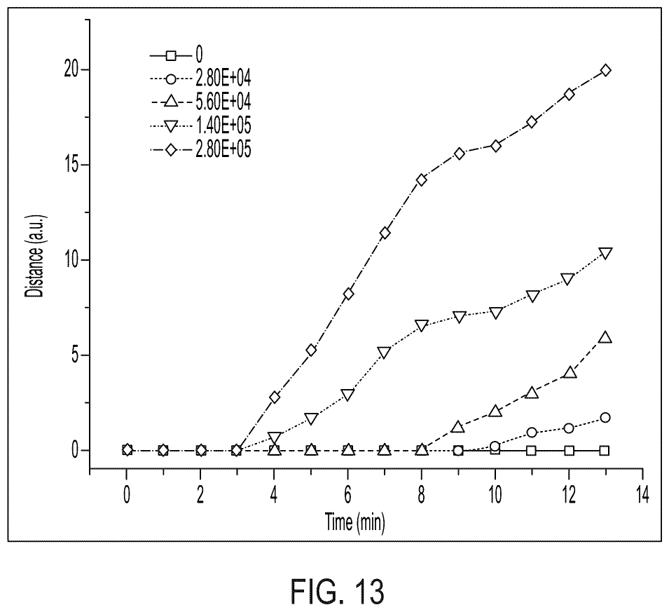

To evaluate the relationship between ink advancement and PtNPs concentration, PtNPs solutions were directly loaded in the reaction chamber for an oxygen generation test. FIG. 9a shows the ink advancement distances in the device, pushed by oxygen generated as a result of different numbers of PtNPs reacting with H₂O₂ for 12 min. A plot of time-dependent ink advancement distances is shown in FIG. 13. As the number of PtNPs increases, the ink advancement distance in the device increases, which correlates with the density and size of bubbles in the reaction chamber. In FIG. 9b, the ink advancement distance is linearly correlated with the number of PtNPs in 30% H₂O₂ (r²=0.99, three parallel measurements of each concentration). These indicate that the LFA ruler is sensitive and can detect as low as twenty thousand PtNPs, and also has a wide dynamic range.

### Quantitation of PSA lateral flow strips

To demonstrate the feasibility of LFA ruler with distance-based readout for target quantitation, PSA was used as the model analyte. Commercially available PSA lateral flow strips generate only qualitative or semi-quantitative results. For example, “See Now” PSA Strip (Camp Medica, Romania), Accu-Tell® One Step PSA Serum Test (AccuBio Tec Co. Ltd., China), and Home Prostate Test (Home Health (UK) Ltd., UK) provide qualitative tests with a cut-off value of 4.0 ng/ml; One Step PSA Rapid Test (Biogate Laboratories Ltd., Canada) has a cut-off value of 4 ng/mL and a reference value of 4 ng/ml; OnSite PSA Semi-quantitative Rapid Test (CTK Biotech Inc., USA) provides semi-quantitative tests with a cut-off value of 4 ng/ml and a reference value of 10 ng/mL.

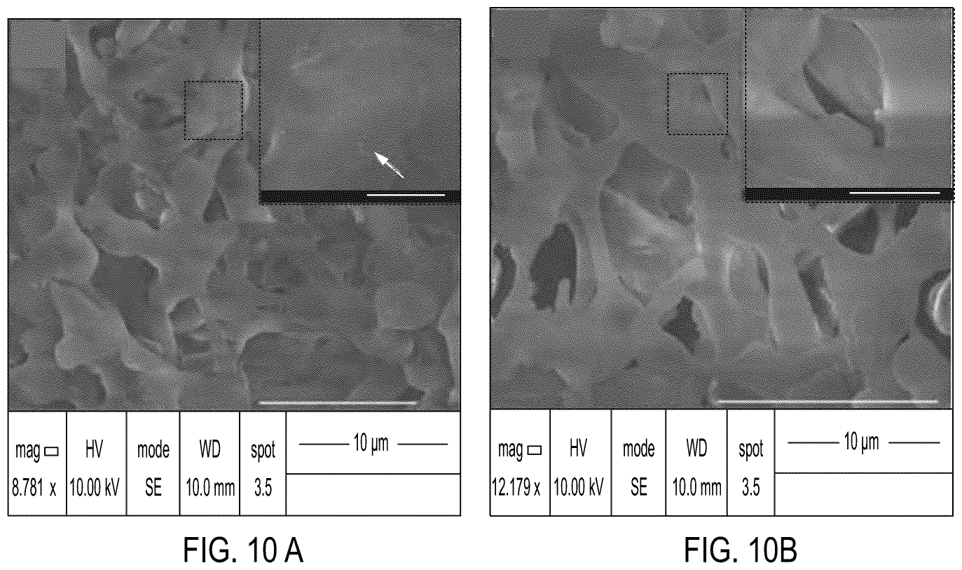

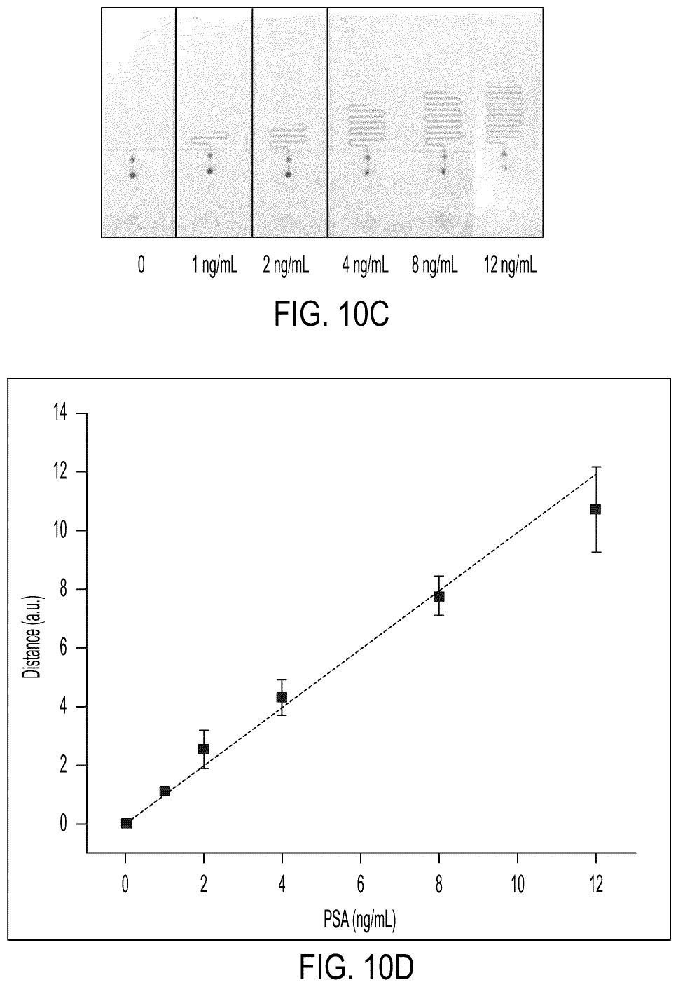

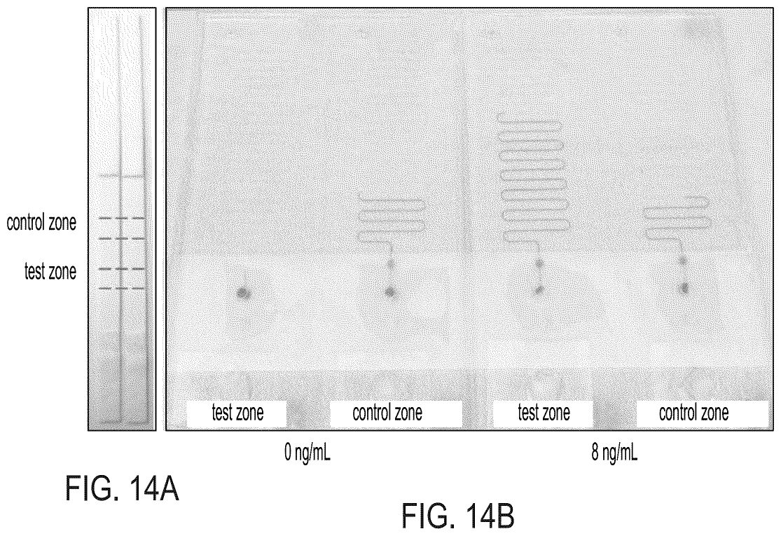

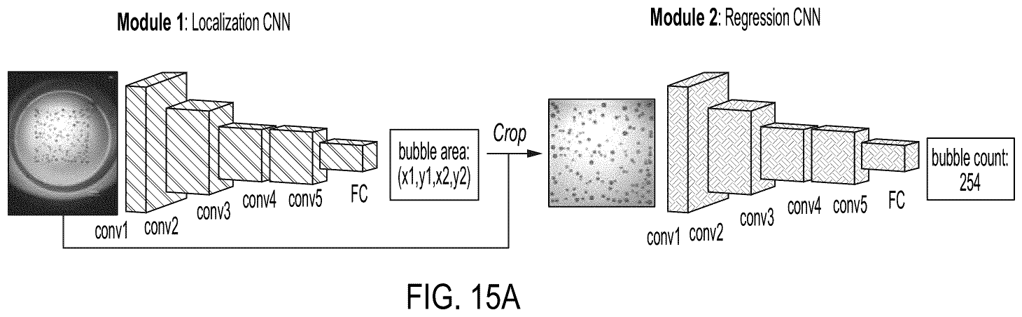

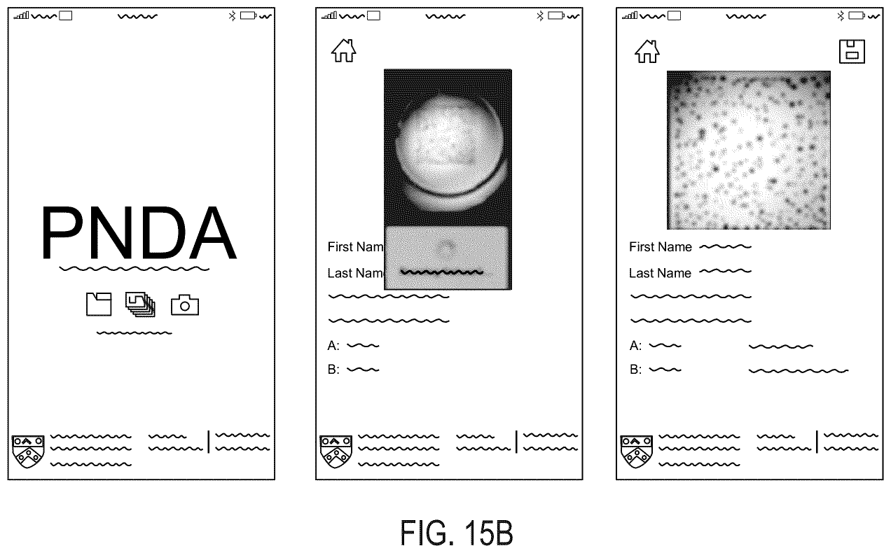

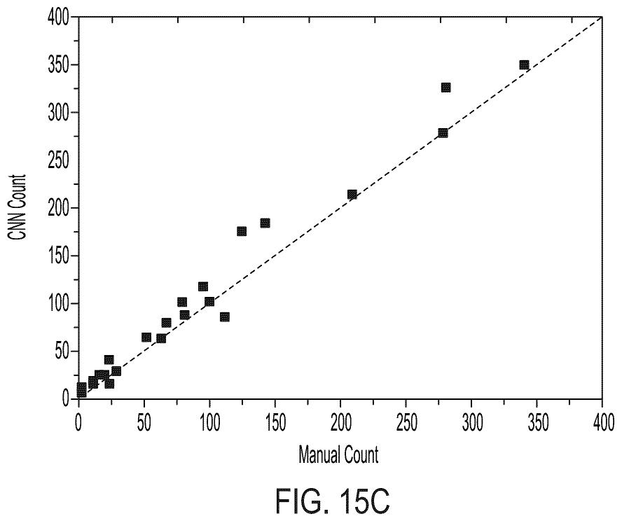

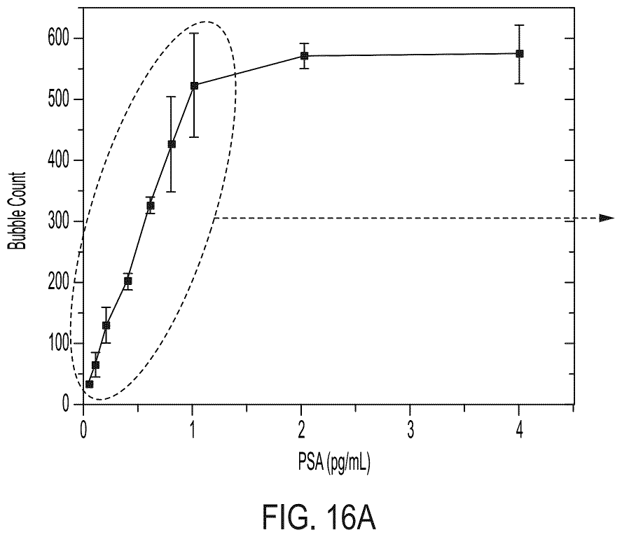

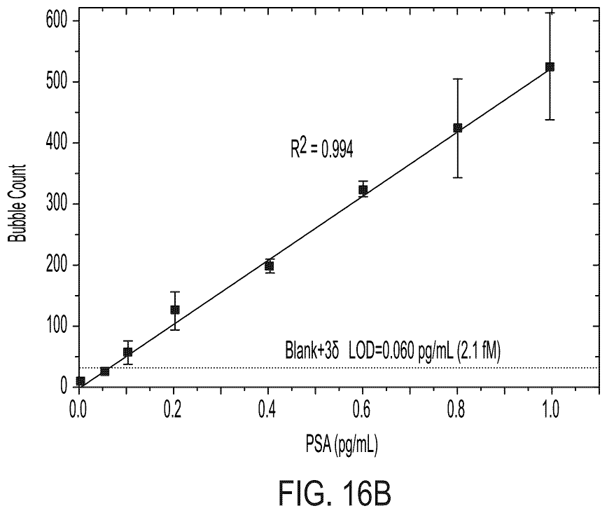

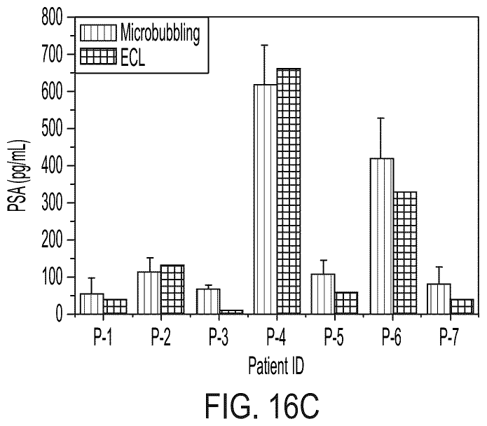

In order to meet the testing requirements for using PSA as a prostate cancer screening biomarker, there is an unmet need to overcome the shortcomings of colorimetric readout of LFA strips and generate quantitative PSA results in concentrations <4 ng/ml, 4-10 ng/mL, and >10 ng/mL, which places patients with respect to clinical decision thresholds but is currently only achievable in the central clinical laboratory setting. To achieve this with the LFA ruler, anti-PSA capture Ab and anti-mouse IgG Ab were pre-immobilized on the surface of the nitrocellulose membrane in the test zone and control zone, separately. The test zone pad and control zone pad from positive strip (PSA, 8 ng/mL) and blank strip were cut and simultaneously tested in LFA rulers (FIG. 14A); FIG. 14B provides ink advancement distances of the test/control zone from the blank strip (0 ng/mL PSA) and the positive strip (8 ng/mL PSA) is significantly different in the LFA rulers. The ink advancement distance of test zone from positive strip is much longer than that from blank strip. Furthermore, there is no significant difference in ink advancement distance between the two control zone pads. FIGS. 10A and 10B show scanning electron microscope images of the test zone pads from positive strip and blank strip, respectively. There are some PtNPs in the cavities of test zone pad from positive strip, which are identified by a green arrow in FIG. 10A; and there are almost no PtNPs in the cavities of test zone pad from blank strip (FIG. 10B). The ink advancement distances in the LFA ruler with different PSA concentrations are shown in FIG. 10C. The linear correlation between ink advancement distances with PSA concentrations is shown in FIG. 10D, tested in three parallel measurements. The calibration equation was y=0.99x+0.04, with a correlation coefficient (r²) of 0.99. The limit of detection (LOD) was calculated to be 0.54 ng/mL, extrapolated by the mean concentration of blank samples (n=3) plus the standard deviation. From the operation standpoint, the time length for reactions on the LFA strip is 15 min, and the subsequent time length on the LFA ruler is 12 min. Thus, the entire testing time is approximately 30 min, which is highly practical in the clinical setting.

### Validation Against Clinical Standard Using Clinical Serum Samples

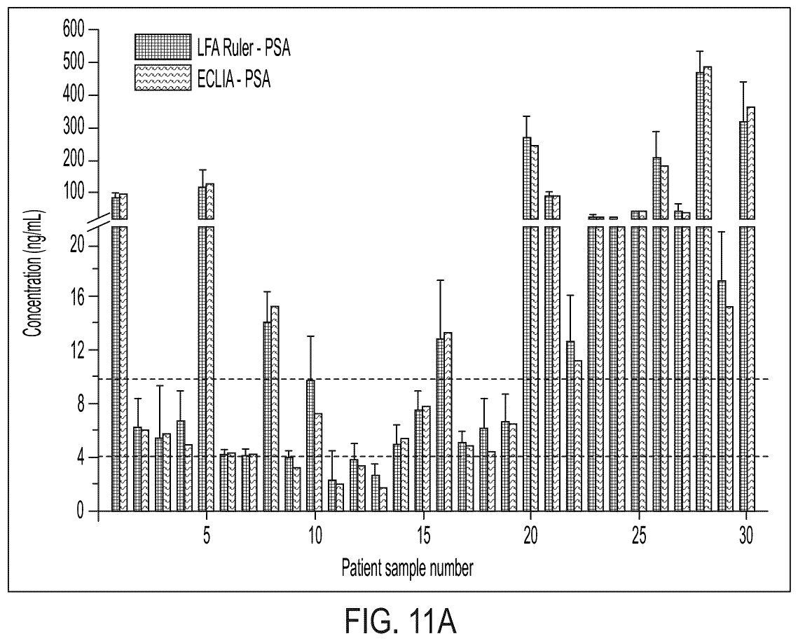

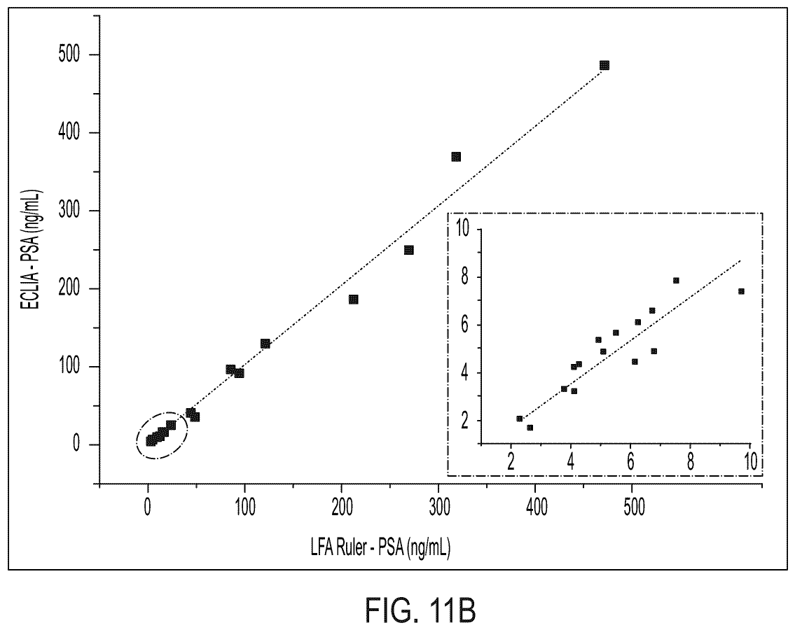

To validate the performance of the LFA ruler against gold-standard clinical assays, PSA concentrations in clinical serum samples (n=30) were quantitated using both the LFA ruler and an FDA-approved ECLIA method (Roche Elecsys Cobas Total PSA assay). The comparison of the results is shown in FIG. 11A. Compared to the clinical results, all of the LFA results remained within the same clinical decision zones (<4 ng/ml, 4-10 ng/ml and >10 ng/mL). FIG. 11B shows a linear relationship between the two analysis methods with an r2 value of 0.92. (r2=0.95 in the inset, for PSA concentrations below 12 ng/mL). These data suggested that the LFA ruler shows excellent agreement with the clinical gold-standard method.

The LFA ruler is the first time that ink advancement signal is used in LFA quantitation, with a simple, robust and portable microfluidic chip. Unlike previous LFA quantitative readout methods, the LFA ruler achieves direct visualized quantitation of assay results with no need for external instruments, such as optical strip reader, fluorescent reader, chemiluminescence reader, magnetic reader, or pressure meter, therefore much more convenient for quantitative and rapid testing. In addition, the relative wider test zone replaces the test line in traditional LFA, in which the thin test line is necessary for colorimetric need. The increased width of “test zone” provides longer interaction time between target molecules and capture antibodies, which has a positive effect in increasing the capture efficiency and assay sensitivity. Coupled with PtNPs' excellent catalytic ability for signal amplification, the sensitivity of this platform is comparable to or better than the best commercially available PSA LFA strips, where gold nanoparticles or other colored labels are used to obtain colorimetric signals for qualitative or semi-quantitative readout. For example, the Instant-view® PSA Whole Blood/Serum Test has an analytical sensitivity of 1 ng/ml (Alfa Scientific Designs, Inc., CA, USA).

The microfluidic chip of a LFA ruler is inexpensive and easy to prepare based on common materials, PDMS and glass. The effect of gas permeability of PDMS is not significant for the open-ended device. The surface of PDMS is smooth and easy to seal with adhesive tapes. There is almost no ink advancement of the blank control experiment, demonstrating the effectiveness of this method. PDMS can be replaced with plastics, e.g., such as poly (methyl methacrylate), which can be processed by laser beam and hot embossing. Thus, there is not always a need for a balance reservoir and other chambers can be sealed simultaneously by one piece of tape.

The LFA ruler offers the potential to quantitatively, sensitively and rapidly assess PSA without any other equipment, with accuracy comparable to clinical gold-standard methods. This platform can be extended to other applications. More analytes and more “ruler” channels can be added to the device to achieve multiplexed quantitation. For testing in resource limited settings, access to centrifuges to get serum from blood samples is not so convenient though there have been some portable centrifuges. A whole blood sample can be tested directly by integrating a filter paper pad into the LFA strip or using commercial blood separators based on filter paper.

### Summary

Provided here is an “LFA ruler” for the quantitative and rapid detection of LFA strips instrument-free. The “LFA ruler” is a PDMS-glass hybrid microfluidic chip with distance-based readout. This platform takes advantage of the convenience of LFA strips, the excellent catalytic ability of PtNP-based signal amplification reporter, as well as the high sensitivity of microfluidic chip. The prototype LFA ruler was capable of rapidly quantitate PSA within 30 min with an LOD of 0.54 ng/mL. The on-chip testing results showed good agreement with those confirmed by an ECLIA method. Compared with conventional LFA techniques, the LFA ruler enables quantitative and sensitive detection of analytes by the naked eye, without need for any instruments and complex operations, which is especially suitable for low-cost quantitation in, as but some example settings, clinical diagnostics, drug screening, food safety, and environmental monitoring.

### Illustrative Disclosure—SARS-COV-2

### Materials

Bovine serum albumin (BSA, A7906-50G), TWEEN® 20 (Molecular Biology Grade, P9416-100ML) and Nunc® MicroWell™ 96 well polystyrene plates (P7366-1CS) were purchased from Sigma-Aldrich, Inc. (St. Louis, MO, USA). Sylgard™ 184 (24236-10) was purchased from Electron Microscopy Sciences (Hatfield, PA, USA). EZ-Link NHS-Biotin, (PI20217), ZebaTM spin desalting columns (89882), disposable standard biopsy punches (6 mm, 12-460-412), sodium azide (S2271100), Tris-HC buffer (1M, pH 8.0, 15568025), magnetic 96-well separator (A14179), Neodymium Disc Magnets (Grade: 35, S430471), hydrogen peroxide (30% in water, BP2633500), PierceTM premium grade Sulfo-NHS (PG82072), PierceTM premium grade EDC (PG82079), NeutrAvidin Protein (PI31000), sodium citrate (78-101-KG), and Fisherbrand™ cover glasses (squares No. 1.5 18 mm, 12541A) were purchased from Thermo Fisher Scientific, Inc. (Rockford, IL, USA). LodeStars® High Bind Carboxyl magnetic beads (trial pack) were purchased from Agilent Technologies, Inc. (Santa Clara, CA, USA). Phosphate-buffered saline (PBS) tablets (T9181), pH 7.4, magnetic stand (631964) were purchased from Clontech Laboratories, Inc. (Mountain View, CA, USA). Antibodies raised against antigens (N-protein or S-Protein) from SARS-CoV-2 (40143-R001 (N1), 40143-R040 (N2), 40143-R004 (N3), 40143-MM05 (N4), 40150-D002 (S1), 40150-D001 (S2), 40150-D003 (S3), 40150-D004 (S1)), antibodies raised against N-protein from SARS-COV (40143-T62 (NP), recombinant N-Protein (40588-V08B) and recombinant S-Protein (40591-V08H3) were purchased from Sino Biological Inc. (Wayne, PA, USA). MES Buffer (50 mM, pH 6.0, 21420006-1) was purchased from Spectrum Chemical Manufacturing Corp. (New Brunswick, NJ, USA). Platinum Nanoparticles (140 nm, tannic acid surface) were purchased from Nanocomposix, Inc. (San Diego, CA, USA). KMPR Applications® 1050 photoresist, SU-8 developer were purchased from MicroChem Corp. (Westborough, MA, USA). Silicon wafers (452, 100 mm, 500um) were purchased from Aidmics Biotechnology Co., LTD. (UniversityWafer) (Boston, MA, USA). The uHandy Mobilephone Microscope (Duet set) was purchased from Aidmics Biotechnology Co. (Taipei, Taiwan, China).

### Clinical Swab Samples

Nasopharyngeal swab samples were collected from patients according to standard operating procedure at the Hospital of University of Pennsylvania, and transported to the clinical laboratory in viral transport media. The samples were tested using the Cepheid GeneXpert SARS-COV-2 test. Deidentified residual samples were stored at 4° C., and subsequently tested using the microbubbling digital antigen assay. The study was approved by the Institutional Review Board of the University of Pennsylvania.

### Inactivated Virus Culture Sample

Supernatant from SARS-COV-2 culture in the BSL3 laboratory at University of Pennsylvania was inactivated by heating at 56° C. for 1 hour, aliquoted and frozen at −20° C. The supernatant is determined to have a virus concentration of 10⁷ genome copies/mL. Aliquots were thawed, diluted and tested using the microbubbling digital antigen assay.

### Fabrication of Microbubbling Microchips

Briefly, a cover glass as the bottom supporting layer, a PDMS sheet with an array of micro wells as the middle layer, and a PDMS top layer containing a round sample chamber was assembled together. Then the assembled microbubbling microchips were coated with a 3 μm thick parylene C layer on the surface.

### Functionalization of Superparamagnetic Microbeads and Platinum Nanoparticles

Similar Protocol was Applied to Functionalize Superparamagnetic Microbeads and platinum nanoparticles as previously reported Briefly, LodeStars® High Bind 2.7-μm diameter carboxyl-terminated superparamagnetic beads were functionalized with antibody using EDC-NHS coupling, blocked with 1% BSA, and then resuspended in 1 mL PBS buffer pH 7.4 containing 1% BSA and 0.02% sodium azide for storage at 4° C.

NeutrAvidin was coated on the surface of PtNPs through sulfo-Pt bonds by simply mixing them in citrate buffer overnight at 4° C., blocked with 1% BSA, and then resuspended and stocked in PBS, pH 7.4, containing 1% BSA.

### Microbubbling Digital Antigen Assay

Sample (100 μL) was incubated with suspensions of 500,000 capture antibody functionalized magnetic beads, on a roller (12 rpm) at 23° C. for 2 h. The beads were then separated using a strong magnets and washed 3 times with 300 μL of PBS buffer pH 7.4 containing 1% BSA and 0.01% TWEEN® 20, and then resuspended in 100 μL of 150 ng/ml biotinylated detection antibody in PBS containing 1% BSA, on a roller (12 rpm) at 23° C. for 1 h. The beads were then separated using strong magnets and washed 3 times with 300 μL of PBS buffer pH 7.4 containing 1% BSA and 0.01% TWEEN® 20, and then resuspended in 100 μL of 1 μg/mL NeutrAvidin functionalized PtNP in PBS containing 1% BSA, on a roller (12 rpm) at 23° C. for 30 min. The beads were then separated using strong magnets and then resuspended in 100 μL of 30% H2O2. The magnetic beads slurries were then applied into the chambers of the microbubbling microchips. Then the microbubbling microchips were placed on neodymium disc magnets for 1 min to pull down the beads to the bottom of the microchips. Finally, within 10 min, different number of microbubbles with diameter ranging from 20 μm to 60 μm werantigene observed in the microwell arrays with either microscope or cell phone.

### Imaging and Analysis of Microbubbling Assay Output

The microbubbles on the microbubbling microchips were imaged with iPhone 11 with the uHandy mobilephone microscope (9×, 5 mm focusing length, Aidmics Biotechnology Co. Taipei, Taiwan, China), followed by analysis with NIH ImageJ 1.43U (Dr. Wayne Rashand, National Institutes of Health, USA). For manual counting, the images were first loaded in ImageJ, and then analyzed with the assistance of “cell counter” function to manually obtain the number of microbubbles.

### Results

### Construction of the Assay

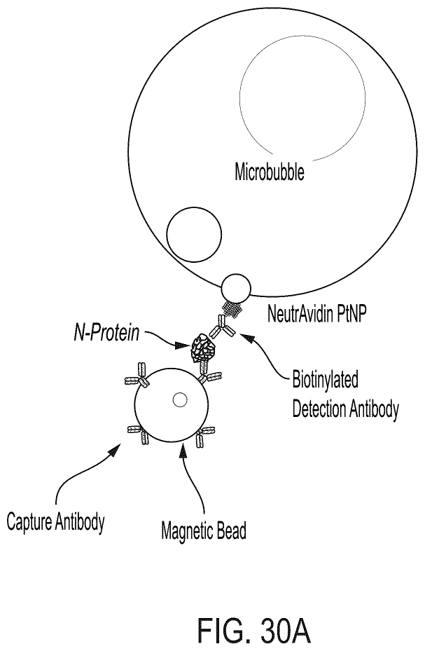

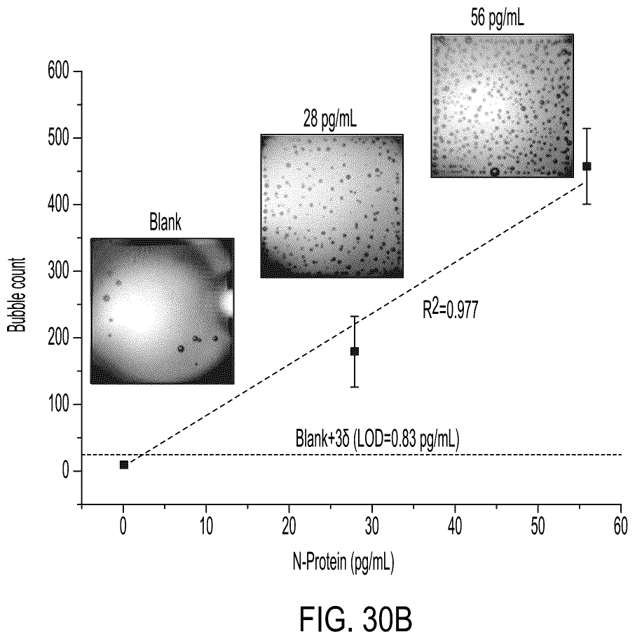

Different antibodies to the SARS-COV-2 N antigen were screened with various capture/detection combinations, using the microbubbling digital assay (data not shown). The pair that generated the highest analytical sensitivity was chosen to construct the assay (FIG. 30A).

### Limit of Detection (LOD) for Recombinant N Antigen

Recombinant N antigen was diluted to different concentrations and tested using the microbubbling digital antigen assay constructed above. The number of bubbles generated was plotted against the N antigen concentration (FIG. 30B). Using blank +3 standard deviation of the blank, the limit of detection (LOD) for recombinant N antigen was determined to be 0.83 pg/mL.

### LOD for Inactivated SARS-COV-2 Virus

Supernatant from SARS-COV-2 culture was inactivated using heat, diluted to different concentrations and tested using the microbubbling digital antigen assay. The number of bubbles generated was plotted against the virus concentration (FIG. 2). Using blank +3 standard deviation of the blank, the LOD for inactivated SARS-CoV-2 virus was determined to be 85 copies/mL.

### Clinical Sample Testing

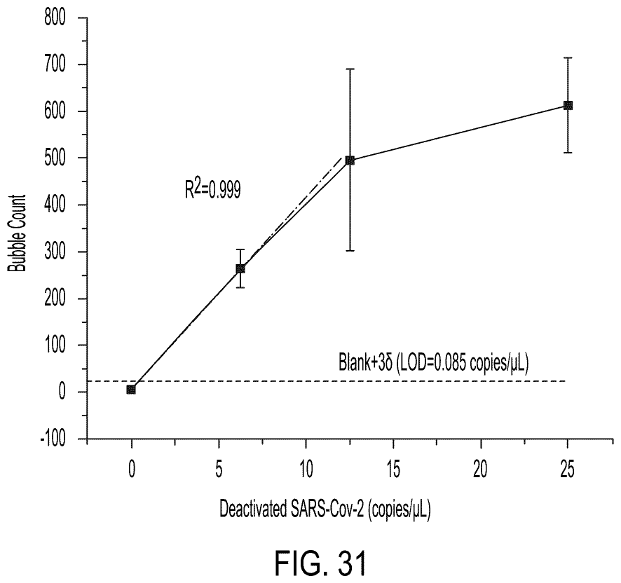

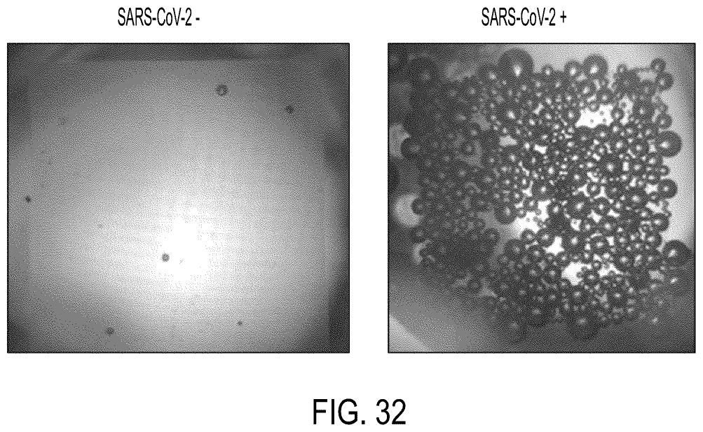

Deidentified clinical nasopharyngeal swab samples were tested using the microbubbling digital antigen assay, and results were compared to clinical testing results using the Cepheid GeneXpert SARS-COV-2 rRT-PCR method (LOD 250 copies/mL). Samples with bubble count equal to or above the count generated at LOD in FIG. 31 were determined to be positive, and those with bubble count below the LOD were determined to be negative. Examples of clinical sample testing results are shown in FIG. 32.

### Discussion

As shown, a microbubbling digital assay can be applied to the detection of SARS-COV-2 N antigen with high analytical sensitivity, with LOD comparable to, or better than many rRT-PCR assays. Clinical sample testing comparing to an FDA EUA-approved rRT-PCR test showed excellent clinical sensitivity and specificity. This indicates that with additional clinical validation, the SAR-COV-2 microbubbling digital antigen assay can be a valuable tool to aid in SARS-COV-2 diagnostic testing. The assay can be applied to other viral antigens, and in other matrices such as nasal swabs, saliva, and the like. With the convenient computer vision-based algorithm on smartphone for bubble counting, this assay can also be used at the point-of-care.

Besides diagnostic use, the assay is also useful in advancing understanding of the SARS-COV-2 virus. It is noteworthy that very little is known about the concentration and dynamics of SARS-COV-2 antigens in different body fluids during the infection course. The microbubbling digital assay can be used as a quantitative assay to probe the concentration and dynamics of various virus antigens at different stages of infection, and can also be used post-vaccination to examine the dynamics of antigen vaccines after vaccination.

### Illustrative Embodiments

Provided here are illustrative Embodiments of the disclosed technology. p These embodiments are illustrative only and do not limit the scope of the present disclosure or of the claims attached hereto.

Embodiment 1. A method, comprising: contacting an analyte, a promoter tag, and an anchor, the promoter tag being configured to bind to the analyte, the promoter tag further comprising a reaction promoter, the anchor being configured to bind to the analyte, the contacting being performed under conditions such that the promoter tag binds with the analyte and the anchor binds with the analyte so as to form a complex; contacting the complex with a reaction substrate so as to evolve a reaction product; and detecting at least some of the reaction product.

The disclosed methods can be applied to any analyte. The disclosed methods are especially well-suited to biological analytes, such as, e.g., antibodies, antigens, cells, cell components, nucleic acids, and the like.

Without being bound to any particular theory, the disclosed methods can use a so-called “sandwich” assay; such assays are well-known in the context of ELISA assays. In such an assay, the promoter tag binds to the analyte, and the anchor also binds to the analyte so as to form a complex. (Unreacted analyte, promoter tag, and anchor can be washed away, as is known to those of skill in the art.) The complex is then reacted to as to form a reaction product (e.g., a gas) that is then detected.

Without being bound to any particular theory, the disclosed methods can be performed in solution (i.e., without immobilizing any of the analyte, the promoter tag, or the anchor). Following contact between the analyte, promoter tag, and anchor (and complex formation), the complex can be directed to a location, e.g., on a substrate and immobilized there). In this way, non-complexed analyte, promoter tag, and anchor remains in solution and can be washed away, leaving behind only complexes that have been directed to a location and immobilized at that location.

Embodiment 2. The method of Embodiment 1, further comprising applying a gradient so as to direct the complex to a location. Such gradients include, e.g., a magnetic field, an electrical field, a pressure field, a chemical gradient, fluid motion, or any combination thereof. Magnetic fields are considered especially suitable, and can be used with, e.g., an anchor that includes a portion that is ferromagnetic.

Embodiment 3. The method of Embodiment 1, further comprising applying a gradient so as to direct the anchor to a location. Without being bound to any particular theory, this can be performed to direct an anchor to a location before the anchor is contacted with the analyte, though this is not a requirement.

Embodiment 4. The method of any one of Embodiments 2-3, wherein the gradient comprises a magnetic field, an electric field, a pressure field, or any combination thereof.

Embodiment 5. The method of any one of Embodiments 2-4, wherein the location is a location on a substrate. A substrate can be planar, curved, porous, non-porous, tubular, polygonal, or any combination thereof.

Embodiment 6. The method of any one of Embodiments 2-4, wherein the location is a location within a depression of a substrate.

Embodiment 7. The method of any one of Embodiments 1-6, wherein the promoter tag comprises an antibody complementary to the analyte, a nucleic acid complementary to the analyte, an aptamer complementary to the analyte, a nanobody complementary to the analyte, an affinity peptide complementary to the analyte, a molecular imprinting polymer complementary to the analyte, a ligand complementary to the analyte, a small molecule complementary to the analyte, a drug complementary to the analyte, or any combination thereof. Exemplary analyte-complementary portions include, without limitation, PSA, troponin, HIV antigen P24, BhcG, CRP, tumor markers (e.g., AFP, CA19-9, CA-125, CA15-3, CEA, HE4), cytokines, infectious bacterial/viral antigens, neurological disease biomarkers (e.g., Tau, aβ40, aβ42) and drugs of abuse.

Embodiment 8. The method of any one of Embodiments 1-7, wherein the promoter tag comprises a catalyst portion.

Embodiment 9. The method of Embodiment 8, wherein the catalyst portion comprises a metal, an enzyme, a metal oxide, a transition metal, a lanthanide, or any combination thereof. A catalyst portion can include platinum. A catalyst portion can also include one or more of HRP, catalase, gold, a heavy metal, manganese dioxide (MnO₂), lead dioxide (PbO₂), iron (III) oxide (Fe₂O₃) or other oxides. A catalyst portion can include a transition metal, a lanthanide, or any combination of these.

Embodiment 10. The method of any one of Embodiments 1-9, wherein the anchor comprises a moiety complementary to the analyte. Suitable moieties include, e.g., antibodies complementary to the analyte, nucleic acids complementary to the analyte, aptamers complementary to the analyte, nanobodies complementary to the analyte, affinity peptides complementary to the analyte, molecular imprinting polymers complementary to the analyte, ligands complementary to the analyte, a small molecule complementary to the analyte, drugs complementary to the analyte, or any combination thereof. Exemplary analyte-complementary portions include, without limitation, PSA, troponin, HIV antigen P24, BhcG, CRP, tumor markers (e.g., AFP, CA19-9, CA-125, CA15-3, CEA, HE4), cytokines, infectious bacterial/viral antigens, neurological disease biomarkers (e.g., Tau, aβ40, aβ42) and drugs of abuse.

Embodiment 11. The method of any one of Embodiments 1-10, wherein the anchor tag comprises a ferromagnetic portion. Such a ferromagnetic portion can be, e.g., an iron particle, or other particle susceptible to magnetic fields.

Embodiment 12. The method of any one of Embodiments 1-11, wherein the reaction substrate comprises hydrogen peroxide. Hydrogen peroxide is considered especially suitable where the catalyst portion comprises platinum, as platinum can react with hydrogen peroxide to evolve oxygen gas.

Embodiment 13. The method of any one of Embodiments 1-12, wherein the reaction product comprises a gas. Oxygen gas is one suitable gas, but other gases are also suitable. For example, nitrogen gas, hydrogen gas, and other gases can be used.

Embodiment 14. The method of any one of Embodiments 1-13, wherein the detection comprises visual or optical detection.

Embodiment 15. The method of Embodiment 14, wherein the detection is performed manually. As one example, a user can count the number of one or more bubbles evolved at one or more locations on a substrate. A user can also determine the sizes of one or more bubbles evolved at one or more locations on a substrate.

Embodiment 16. The method of Embodiment 14, wherein the detection is performed in an automated fashion. Detection can be performed using a computer, a mobile device (e.g. a smartphone), or by other automated device. Detection can include counting the number and/or sizes of one or more bubbles evolved at one or more locations on a substrate.

Embodiment 17. The method of any one of Embodiments 1-16, further comprising relating the detection of the at least some of the reaction product to a level of the analyte. This can be done by, e.g., comparing a number and/or size of bubbles evolved from a reaction to a calibration standard. As an example, a user can utilize a calibration standard (also known as a “calibration curve,” in some instances ”) that is framed in terms of bubbles/area and that is generated by reacting a substrate (which can be present at a known amount) with catalyst particles present at known densities (i.e., density of particles/area) and recording the number of bubbles/area evolved from the calibration experiments.

Embodiment 18. The method of any one of Embodiments 1-17, wherein one or more of (a) the contacting an analyte, a promoter tag, and an anchor, (b) contacting the complex with a reaction substrate, and (c) detecting at least some of the reaction product is performed in an automated fashion. Further, one or more of sample (analyte) loading, analyte reaction, and washing (e.g., to remove unbound analyte, promoter tag, and/or anchor) can be performed in an automated fashion. For example, addition of analyte, addition of promoter tag and/or anchor, and application of a gradient to direct complexes to one or more locations on a substrate can be performed in an automated fashion.

A gradient can be applied to direct complexes (and/or anchor) to one or more locations on a substrate. For example, a gradient can be applied to direct a first population of complexes to one or more locations on a first quadrant of a substrate. A gradient can be applied to direct a second population of complexes to one or more locations on the first quadrant of the substrate or to one or more locations on a second quadrant of a substrate. One or more substrates can be introduced so as to react with the complexes. In this way, a first substrate that is reactive to one or both of the first population of complexes can be introduced, allowing a user to determine the presence/level of the analyte that is associated with the first population of complexes. If the first substrate is reactive to the second population of complexes, the user can determine the presence/level of the analyte that is associated with the second population of complexes. Alternatively, if the first substrate is not reactive with the second population of complexes, a user can introduce a second substrate that is reactive with the second population of complexes, so as to allow the user to determine the presence/level of the analyte that is associated with the second population of complexes.

Embodiment 19. A method, comprising: contacting a plurality of first analytes, a plurality of second analytes, a plurality of first promoter tags, a plurality of second promoter tags, a plurality of first anchors, and a plurality of second anchors, the first promoter tag being configured to bind to a first analyte, the first promoter tag further comprising a reaction promoter, the first anchor being configured to bind to the first analyte, the second promoter tag being configured to bind to a second analyte, the second promoter tag further comprising a reaction promoter, the second anchor being configured to bind to the second analyte, the contacting being performed under conditions such that the first promoter tag binds with the first analyte and the first anchor binds to the analyte so as to form a first complex; the contacting being performed under conditions such that the second promoter tag binds with the second analyte and the second anchor binds to the analyte so as to form a second complex; contacting the first complex with a reaction substrate so as to evolve a first reaction product; contacting the second complex with a reaction substrate so as to evolve a second reaction product; detecting at least some of the first reaction product; detecting at least some of the second reaction product.

Embodiment 20. The method of Embodiment 19, wherein at least one of the first reaction product and the second reaction product is in gas form. Example gases include, e.g., oxygen, hydrogen, nitrogen, and the like.

Embodiment 21. The method of any one of Embodiments 19-20, further comprising applying a gradient (a) so as to direct the first anchor to a location, (b) so as to direct the second anchor to a location, or both (a) and (b).

Embodiment 22. The method of any one of Embodiments 19-20, further comprising applying a gradient (a) so as to direct the first complex to a location, (b) so as to direct the second complex to a location, or both (a) and (b).

Embodiment 23. A system, comprising: an amount of a first promoter tag, the first promoter tag being configured to bind to a first analyte, the first promoter tag further comprising a first reaction promoter, an amount of a first anchor, the first anchor being configured to bind to the first analyte and the first anchor further comprising a ferromagnetic portion; a substrate; and a gradient source configured to exert a force on the ferromagnetic portion of the first anchor.

Exemplary analytes, promoter tags, and anchors are described elsewhere herein, as are exemplary substrates. Suitable gradient sources include, e.g., pressure sources, magnetic field sources, and the like.

Embodiment 24. The system of Embodiment 23, further comprising an amount of a second promoter tag, the second promoter tag being configured to bind to a second analyte, the second promoter tag further comprising a second reaction promoter, an amount of a second anchor, the second anchor being configured to bind to the second analyte and the second anchor further comprising a ferromagnetic portion.

Embodiment 25. The system of any one of Embodiments 23-24, wherein the substrate comprises a plurality of depressions, and wherein the gradient source is configured to direct the first anchor to a location within a depression. Depressions can be of the same or different sizes. Depressions can be arrayed in a periodic fashion on a substrate. Without being bound to any particular theory, depressions can be spaced relative to one another so as to reduce or eliminate coalescence between bubbles that may form at adjacent or otherwise nearby depressions.

Likewise, complexes and/or anchors can be directed to substrate locations that are positioned relative to one another so as to reduce or eliminate coalescence between bubbles that may form at adjacent or otherwise nearby substrate locations.

Embodiment 26. The system of any one of Embodiments 23-25, further comprising a detector configured to detect a product of a first reaction related to contact between the first reaction promoter and a reaction substrate.

Embodiment 27. The system of any one of Embodiments 23-26, further comprising a detector configured to detect a product of a second reaction related to contact between the second reaction promoter and a reaction substrate. Example detectors include, e.g., imagers (e.g., CCD devices), PMT devices, and the like.

Embodiment 28. The system of Embodiment 27, wherein the detector is configured to detect the product of the first reaction in an automated fashion.

Embodiment 29. The system of Embodiment 27, wherein the detector is configured to detect the product of the second reaction in an automated fashion.

Embodiment 30. The system of any one of Embodiments 23-29, wherein the system is configured to perform in an automated fashion at least one of (a) contacting the first promoter tag to the first analyte, and (b) contacting the first anchor to the first analyte.

Embodiment 31. The system of any one of Embodiments 24-29, wherein the system is configured to perform in an automated fashion at least one of (a) contacting the second promoter tag to the second analyte, and (b) contacting the second anchor to the second analyte.

Embodiment 32. The system of any one of Embodiments 23-31, wherein the system is configured to operate the gradient source in an automated fashion.

Embodiment 33. A method, comprising: contacting an analyte and a promoter tag, the promoter tag being configured to bind to the analyte, the promoter tag further comprising a reaction promoter, the contacting being performed under conditions such that the promoter tag binds with the analyte so as to form a first complex; contacting the first complex with a capture tag linked to a physical substrate so as give rise to an anchored complex at an anchored complex location on the physical substrate; contacting the anchored complex with a reaction substrate so as to evolve a reaction product that advances an indicator material; and detecting a displacement of the indicator material.

Embodiment 34. The method of Embodiment 33, wherein the indicator material comprises a fluid.

Embodiment 35. The method of Embodiment 34, wherein the fluid is non-transparent, comprises a colorant, or both. Inks, dyes, and the like are all considered suitable. An indicator material can be immiscible with the reaction product, e.g., immiscible with oxygen gas.

Embodiment 36. The method of any one of Embodiments 33-35, further comprising transporting the anchored complex to a reaction chamber.

Embodiment 37. The method of any one of Embodiments 33-36, further comprising physically separating a portion of the physical substrate that comprises the anchored complex location from the remainder of the physical substrate. Physical separation can be accomplished by cutting, tearing, and the like.

Embodiment 38. The method of any one of Embodiments 33-37, further comprising correlating the displacement of the indicator with a presence of the analyte. As an example, one can correlate the displacement of the indicator upon reaction of a sample with the displacement of the indicator evolved from a known sample.

Embodiment 39. The method of any one of Embodiments 33-38, wherein the reaction product comprises a fluid.

Embodiment 40. The method of Embodiment 39, wherein the reaction product comprises a gas. Suitable gases are described elsewhere herein and can include, e.g., oxygen gas or other gases evolved from reaction of a substrate with a catalytic material.

Embodiment 41. The method of any one of Embodiments 33-40, further comprising contacting a second analyte and a second promoter tag, the second promoter tag being configured to bind to the second analyte, the second promoter tag further comprising a second reaction promoter, the contacting being performed under conditions such that the second promoter tag binds with the second analyte so as to form a second complex; contacting the second complex with a second capture tag linked to a physical substrate so as give rise to an anchored second complex at a second anchored complex location on the physical substrate; contacting the second anchored complex with a second reaction substrate so as to evolve a second reaction product that advances a second indicator material; and detecting a displacement of the second indicator material.

The method of the foregoing embodiments can be performed in a multiplexed fashion, i.e., to detect the presence of two or more analytes using one, two, or more channels. For example, the methods could be applied to detect the presence of a first analyte based on displacement of an indicator along a first channel and the presence of a second analyte based on displacement of an indicator along a second channel. It should be understood that the methods can be performed using a single reaction substrate (e.g., H2O2) that evolves reaction products that displace indicator material in multiple channels, e.g., with different channels corresponding to different analytes.

Embodiment 42. A system for detecting an analyte, comprising: a reaction chamber configured to receive one or more of a sample and a substrate; an indicator chamber in fluid communication with the reaction chamber, an amount of indicator material optionally disposed within the indicator chamber; and an indicator channel in fluid communication with the indicator chamber, the indicator channel optionally comprising one or more bends, the indicator channel configured to accommodate displaced indicator material that is displaced by evolution of a reaction product in the reaction chamber that effects displacement of the indicator material.

Embodiment 43. The system of Embodiment 42, further comprising a capture strip, the capture strip comprising a capture region that comprises a capture tag configured to bind an analyte so as to immobilize the analyte at the capture region of the capture strip.

Embodiment 44. The system of Embodiment 42, wherein the capture strip is pervious. Porous, fibrous, and other pervious or wicking materials are all considered suitable.

Embodiment 45. The system of Embodiment 42, wherein the capture strip is porous.

Embodiment 43. The system of Embodiment 43, wherein the capture region is configured to be removable from the capture strip. The capture region can be cut, torn, or otherwise removed from the capture strip.

Embodiment 47. The system of Embodiment 43, wherein the capture region is configured to be insertable into the reaction chamber.

Embodiment 48. The system of any one of Embodiments 42-47, further comprising a balance chamber in fluid communication with the reaction chamber and the indicator chamber.

Embodiment 49. The system of any one of Embodiments 42-48, wherein the indicator channel comprises one or more indicia. Suitable indicia can be used to mark one or more distances along the length of the indicator channel.

Embodiment 50. The system of any one of Embodiments 42-49, further comprising a supply of a promoter tag configured to bind to the analyte, the promoter tag further comprising a reaction promoter configured to evolve a reaction product upon reaction of the reactor promotor with a reaction substrate.

Embodiment 51. The system of any one of Embodiments 42-50, wherein the indicator material comprises a fluid.

Embodiment 52. The system of any one of Embodiments 42-51, wherein the system comprises one or more of (a) a second reaction chamber configured to receive one or more of a sample and a substrate, (b) a second indicator chamber in fluid communication with the second reaction chamber, (c) an amount of a second indicator material optionally disposed within the second indicator chamber, and (d) a second indicator channel in fluid communication with the second indicator chamber, the second indicator channel optionally comprising one or more bends, the second indicator channel configured to accommodate displaced second indicator material that is displaced by evolution of a reaction product in the second reaction chamber that effects displacement of the second indicator material.

As an example, a system can include a second reaction chamber that receives a sample and a substrate, where reaction between the sample and the substrate evolves a second reaction product. The second reaction product can then then displace an amount of (second) indicator material within a second indicator channel. In this way, systems according to the present disclosure can allow for a user to detect multiple analytes by monitoring displacement of indicator material in indicator channels that correspond to each analyte; each indicator channel can be in fluid communication with a different reaction chamber, with each different reaction chamber in turn being designated for use in connection with a different analyte.

Embodiment 53. A method, comprising: reacting a sample comprising an amount of prostate specific antigen (PSA) with a promoter tag configured to bind specifically to PSA under such conditions that the promoter tag binds to the PSA; contacting the sample with an anchor under such conditions that the anchor binds specifically to the PSA, the anchor optionally comprising a magnetizable material, the reacting and contacting being performed so as to give rise to a complex that comprises the PSA, the promoter tag, and the anchor, immobilizing the complex; contacting the complex with a reaction substrate so as to evolve a reaction product; detecting at least some of the reaction product; and correlating detected reaction product with a level of PSA in the sample.

Embodiment 54. A method, comprising: reacting a sample comprising an amount of βhCG with a promoter tag configured to bind specifically to βhCG under such conditions that the promoter tag binds to the βhCG; contacting the sample with an anchor under such conditions that the anchor binds specifically to the βhCG, the anchor optionally comprising a magnetizable material, the reacting and contacting being performed so as to give rise to a complex that comprises the βhCG, the promoter tag, and the anchor, immobilizing the complex; contacting the complex with a reaction substrate so as to evolve a reaction product; detecting at least some of the reaction product; and correlating detected reaction product with a level of βhCG in the sample.

Embodiment 55. A kit, comprising: a supply of a promoter tag configured to bind specifically to an analyte, the analyte optionally comprising PSA or βhCG; a supply of an anchor configured to bind specifically to the analyte, the anchor optionally comprising a magnetizable material, and the promoter tag comprising a material configured to evolve a gaseous product when contacted with a reaction substrate under effective conditions.

Embodiment 56. A method, comprising: reacting a sample comprising an amount of an analyte with a promoter tag configured to bind specifically to the analyte under such conditions that the promoter tag binds to the analyte; contacting the sample with an anchor under such conditions that the anchor binds specifically to the analyte, the anchor optionally comprising a magnetizable material (e.g., iron), the reacting and contacting being performed so as to give rise to a complex that comprises the analyte, the promoter tag, and the anchor, immobilizing the complex; contacting the complex with a reaction substrate so as to evolve a gaseous reaction product; detecting at least some of the gaseous reaction product; and correlating detected reaction product with a level of the analyte in the sample.

Embodiment 57. A method, comprising: contacting an analyte, a promoter tag, and an anchor, the analyte being a coronavirus nucleocapsid or a coronavirus spike, the promoter tag being configured to bind to the analyte, the promoter tag further comprising a reaction promoter, the anchor being configured to bind to the analyte, the contacting being performed under conditions such that the promoter tag binds with the analyte and the anchor binds with the analyte so as to form a complex; contacting the complex with a reaction substrate so as to evolve a reaction product, the reaction product optionally being a gas; and detecting at least some of the reaction product.

The promoter tag (and/or the anchor) can include an antibody complementary to the analyte, a nucleic acid complementary to the analyte, an aptamer complementary to the analyte, a nanobody complementary to the analyte, an affinity peptide complementary to the analyte, a molecular imprinting polymer complementary to the analyte, a ligand complementary to the analyte, a small molecule complementary to the analyte, a drug complementary to the analyte, or any combination thereof. Suitable such species will be known to those of ordinary skill in the art.

The promoter tag and/or the anchor can each comprise an antibody (which can be the same antibody or a different antibody) that binds to the analyte. Example antibodies (referenced by catalog number Sino Biological Inc. (Wayne, PA, USA)) can include, e.g., antibodies against antigens N-protein or S-protein from SARS-COV-2 (40143-R001 (N1), 40143-R040 (N2), 40143-R004 (N3), 40143-MM05 (N4), 40150-D002 (S1), 40150-D001 (S2), 40150-D003 (S3), 40150-D004 (S1)), and antibodies raised against N-protein from SARS-COV (40143-T62 (NP).

Embodiment 58. The method of Embodiment 57, wherein the coronavirus is SARS-COV-2.

Embodiment 59. The method of Embodiment 58, wherein the analyte is a nucleocapsid protein or a portion of a nucleocapsid protein of SARS-COV-2.

Embodiment 60. The method of Embodiment 58, wherein the analyte is a spike protein or a portion of a spike protein (e.g., receptor binding domain, RBD) of SARS-CoV-2.

Embodiment 61. The method of any one of Embodiments 57-60, wherein the analyte is collected from a nasopharyngeal sample, a saliva sample, a nasal sample, a urine sample, a stool sample, another body fluid (e.g., blood, sputum, vomit, and the like), or any combination thereof. The methods can be performed on a patient sample taken directly from the patient, but they can also be performed on a patent sample that is first purified, amplified, or otherwise prepared.

Embodiment 62. The method of Embodiment 61, wherein the analyte is collected from a nasal sample.

Embodiment 63. The method of Embodiment 62, wherein the analyte is collected from a saliva sample or from another body fluid.

Embodiment 64. The method of any one of Embodiments 57-63, wherein the sample is taken from an individual (a) known or suspected to be post-infection with SARS-COV-2, (b) known or suspected to have received treatment for SARS-COV-2, (c) known or suspected to have received a vaccine for SARS-COV-2, or any combination of (a), (b), and (c).

Embodiment 65. The method of any one of Embodiments 57-64, wherein the promoter tag comprises an antibody complementary to the analyte, a nucleic acid complementary to the analyte, an aptamer complementary to the analyte, a nanobody complementary to the analyte, an affinity peptide complementary to the analyte, a molecular imprinting polymer complementary to the analyte, a ligand complementary to the analyte, a small molecule complementary to the analyte, a drug complementary to the analyte, or any combination thereof.

Embodiment 66. The method of any one of Embodiments 57-65, wherein the promoter tag comprises a catalyst portion.

Embodiment 67. The method of Embodiment 66, wherein the catalyst portion comprises a metal, an enzyme, a metal oxide, a transition metal, a lanthanide, or any combination thereof.

Embodiment 68. The method of Embodiment 67, wherein the metal comprises platinum and wherein the reaction substrate comprises hydrogen peroxide.

Embodiment 69. The method of any one of Embodiments 57-68, further comprising applying a gradient so as to direct the complex to a location, the anchor optionally being sensitive to the gradient.

Embodiment 70. The method of any one of Embodiments 57-69, wherein the detection comprises visual or optical detection.

Embodiment 71. The method of Embodiment 70, wherein the detection is performed manually.

Embodiment 72. The method of Embodiment 70, wherein the detection is performed in an automated fashion.

Embodiment 73. The method of any one of Embodiments 57-72, further comprising relating the detection of the at least some of the reaction product to a level of the analyte.

Embodiment 74. A kit, comprising: a supply of a promoter tag configured to bind specifically to an analyte, the analyte being a coronavirus nucleocapsid or a coronavirus spike; a supply of an anchor configured to bind specifically to the analyte, the anchor optionally comprising a magnetizable material, and the promoter tag comprising a material configured to evolve a gaseous product when contacted with a reaction substrate under effective conditions.

Embodiment 75. The kit of Embodiment 74, wherein the coronavirus is SARS-COV-2.

Embodiment 76. The kit of Embodiment 75, wherein the analyte is a nucleocapsid protein of SARS-COV-2.

Embodiment 77. The kit of Embodiment 76, wherein the analyte is a spike protein of SARS-COV-2.

Embodiment 78. The kit of any one of Embodiments 74-77, further comprising a translucent portion configured to permit observation of the gaseous product.

Embodiment 79. The kit of any one of Embodiments 74-78, wherein the kit is configured to engage with a portable computing device configured to observe the gaseous product.

Embodiment 80. The kit of Embodiment 79, wherein the portable computing device is a smartphone or a tablet computer.

Embodiment 81. A system, comprising: a module configured to contain a complex and the module comprising a substrate configured to receive the complex, the complex comprising: a first promoter tag, the first promoter tag bound to a coronavirus nucleocapsid or a coronavirus spike present in a sample, the first promoter tag further comprising a first reaction promoter, an amount of a first anchor, the first anchor being bound to the first analyte and the first anchor further comprising a portion sensitive to a gradient, the module being configured to engage with a detector configured to detect, within the module, a gaseous product of the reaction of the first reaction promoter with a reaction substrate.

Embodiment 82. The system of Embodiment 81, wherein the coronavirus is SARS-COV-2.

Embodiment 83. The system of Embodiment 82, wherein the analyte is a nucleocapsid protein or a portion of a nucleocapsid protein of SARS-COV-2.

Embodiment 84. The system of Embodiment 82, wherein the analyte is a spike protein or a portion of a spike protein (e.g., receptor binding domain, RBD) of SARS-CoV-2.

Embodiment 85. The system of any one of Embodiments 81-84, further comprising the detector configured to detect, within the module, the gaseous product of the reaction of the first reaction promoter with the reaction substrate.

Embodiment 86. The system of any one of Embodiments 81-85, the system further comprising a processor configured to detect a level of a gaseous product evolved from the reaction of the reaction promoter and a reaction substrate.

Embodiment 87. The system of Embodiment 86, wherein the processor is configured to relate the level of the gaseous product to a level of the coronavirus in the sample.

Embodiment 88. The system of any one of Embodiments 81-87, wherein the substrate effects application of the gradient.

Embodiment 89. The system of any one of Embodiments 81-88, wherein the system is configured to apply the gradient.

Embodiment 90. The system of any one of Embodiments 81-89, wherein the module comprises one or more of the first promoter tag and the first anchor.

Embodiment 91. A method, comprising: reacting a sample comprising an amount of an analyte with (1) a promoter tag configured to bind specifically to the analyte and (2) an anchor, the reacting being performed such that the anchor and the promoter tag bind to the analyte, the analyte being a coronavirus nucleocapsid or a coronavirus spike, the anchor optionally comprising a magnetizable material, the reacting giving rise to a complex that comprises the analyte, the promoter tag, and the anchor, immobilizing the complex; contacting the complex with a reaction substrate so as to evolve a gaseous reaction product; detecting at least some of the gaseous reaction product; and correlating detected gaseous reaction product with a level of the analyte in the sample.

Embodiment 92. The method of Embodiment 91, wherein the coronavirus is SARS-COV-2.

Embodiment 93. The method of Embodiment 92, wherein the analyte is a nucleocapsid protein of SARS-COV-2.

Embodiment 94. The method of Embodiment 93, wherein the analyte is a spike protein of SARS-COV-2.

Embodiment 95. The method of any one of Embodiments 91-94, wherein the analyte is collected from a nasopharyngeal sample, a nasal sample, a urine sample, a stool sample, another body fluid, or any combination thereof.

### REFERENCES

B. M. Cummins, F. S. Ligler, G. M. Walker, Biotechnol Adv 2016, 34, 161-176.
H. Chen, K. Liu, Z. Li, P. Wang, Clinica Chimica Acta 2019, 493, 138-147.
A. St John, C. P. Price, Clin Biochem Rev 2014, 35, 155-167.
P. Wang, L. J. Kricka, Clin Chem 2018, 64, 1439-1452.
C. Gaydos, J. Hardick, Expert Rev Anti Infect Ther 2014, 12, 657-672.
S. Fredriksson, M. Gullberg, J. Jarvius, C. Olsson, K. Pietras, S. M. Gustafsdóttir, A. Östman, U. Landegren, Nature Biotechnology 2002, 20, 473.
J. M. Nam, C. S. Thaxton, C. A. Mirkin, Science 2003, 301, 1884-1886.
D. M. Rissin, C. W. Kan, T. G. Campbell, S. C. Howes, D. R. Fournier, L. Song, T. Piech, P. P. Patel, L. Chang, A. J. Rivnak, E. P. Ferrell, J. D. Randall, G. K. Provuncher, D. R. Walt, D. C. Duffy, Nat Biotechnol 2010, 28, 595-599.
H. Chen, M. Crum, D. Chavan, B. Vu, K. Kourentzi, R. C. Willson, ACS Appl Mater Interfaces 2018, 10, 31845-31849.
A. S. Basu, SLAS Technol 2017, 22, 369-386.
H. C. Tekin, M. Cornaglia, M. A. Gijs, Lab Chip 2013, 13, 1053-1059.
W. F. Paxton, K. C. Kistler, C. C. Olmeda, A. Sen, S. K. St Angelo, Y. Cao, T. E. Mallouk, P. E. Lammert, V. H. Crespi, J Am Chem Soc 2004, 126, 13424-13431.
Z. Zhu, Z. Guan, D. Liu, S. Jia, J. Li, Z. Lei, S. Lin, T. Ji, Z. Tian, C. J. Yang, Angew Chem Int Ed Engl 2015, 54, 10448-10453.
Y. Song, Y. Zhang, P. E. Bernard, J. M. Reuben, N. T. Ueno, R. B. Arlinghaus, Y. Zu, L. Qin, Nat Commun 2012, 3, 1283.
Y. Song, X. Xia, X. Wu, P. Wang, L. Qin, Angew Chem Int Ed Engl 2014, 53, 12451-12455.
A. Krizhevsky, I. Sutskever, G. E. Hinton, Commun Acm 2017, 60, 84-90.
E. Shelhamer, J. Long, T. Darrell, Ieee T Pattern Anal 2017, 39, 640-651.
K. He, G. Gkioxari, P. Dollar, R. Girshick, IEEE Trans Pattern Anal Mach Intell 2018.
T. D. Laajala, H. Seikkula, F. Seyednasrollah, T. Mirtti, P. J. Bostrom, L. L. Elo, Sci Rep 2016, 6, 36161.
D. Tilki, S. I. Kim, B. Hu, M. A. Dall'Era, C. P. Evans, J Urol 2015, 193, 1525-1531.
L. J. Sokoll, Z. Zhang, D. W. Chan, A. C. Reese, T. J. Bivalacqua, A. W. Partin, P. C. Walsh, J Urol 2016, 195, 330-336.
A. J. Stephenson, P. T. Scardino, M. W. Kattan, T. M. Pisansky, K. M. Slawin, E. A. Klein, M. S. Anscher, J. M. Michalski, H. M. Sandler, D. W. Lin, J. D. Forman, M. J. Zelefsky, L. L. Kestin, C. G. Roehrborn, C. N. Catton, T. L. DeWeese, S. L. Liauw, R. K. Valicenti, D. A. Kuban, A. Pollack, J Clin Oncol 2007, 25, 2035-2041.
S. Shen, H. Lepor, R. Yaffee, S. S. Taneja, J Urol 2005, 173, 777-780.
S. Vesely, L. Jarolim, K. Duskova, M. Schmidt, P. Dusek, M. Babjuk, BMC Urol 2014, 14, 79.
S. Wilkinson, K. Warren, A. Ramsden, A. Matthews, G. Chodak, Urology 2008, 71, 567-572.
R. D. Tendulkar, S. Agrawal, T. Gao, J. A. Efstathiou, T. M. Pisansky, J. M. Michalski, B. F. Koontz, D. A. Hamstra, F. Y. Feng, S. L. Liauw, M. C. Abramowitz, A. Pollack, M. S. Anscher, D. Moghanaki, R. B. Den, K. L. Stephans, A. L. Zietman, W. R. Lee, M. W. Kattan, A. J. Stephenson, Journal of Clinical Oncology 2016, 34, 3648-3654.
A. I. Abushouk, M. Sanei Taheri, P. Pooransari, S. Mirbaha, A. Rouhipour, A. Baratloo, Emerg (Tehran) 2017, 5, e60-e60.
D. N. Greene, R. L. Schmidt, S. M. Kamer, D. G. Grenache, C. Hoke, T. S. Lorey, Clinica Chimica Acta 2013, 415, 317-321.
C. Gnoth, S. Johnson, Geburtshilfe Frauenheilkd 2014, 74, 661-669.
C. Y. Chen, Y. M. Hwu, C. P. Chen, C. C. Chang, Int J Nanomedicine 2015, 10, 2475-2483.
R. Safavieh, D. Juncker, Lab on a Chip 2013, 13, 4180-4189.
J. Gilmore, M. Islam, R. Martinez-Duarte, Micromachines (Basel) 2016, 7, 52.
J. Mok, M. N. Mindrinos, R. W. Davis, M. Javanmard, Proceedings of the National Academy of Sciences 2014, 111, 2110-2115.
Wang, X. et al. Limits of Detection of Six Approved RT-PCR Kits for the Novel SARS-coronavirus-2 (SARS-COV-2). Clin Chem, doi: 10.1093/clinchem/hvaa099 (2020).
Shi, J., Han, D., Zhang, R., Li, J. & Zhang, R. Molecular and serological assays for SARS-COV-2: insights from genome and clinical characteristics. Clin Chem, doi: 10.1093/clinchem/hvaa122 (2020).
Dasheng Li, M., 1 Dawei Wang, PhD,2 Jianping Dong, MM,3 Nana Wang, MM, 1 He Huang, MM, 1 Haiwang Xu, MB, 1 and Chen Xia. False-Negative Results of Real-Time Reverse-Transcriptase Polymerase Chain Reaction for Severe Acute Respiratory Syndrome Coronavirus 2: Role of Deep-Learning-Based CT Diagnosis and Insights from Two Cases. Korean J Radiol. 21, 505-508 (2020).
Wenling Wang, P. Y. X., MD2; Ruqin Gao, MD3; Roujian Lu, MPH1; Kai Han, BS2; Guizhen Wu, MD1; Wenjie Tan, MD, PhD. Detection of SARS-COV-2 in Different Types of Clinical Specimens. JAMA, doi: 10.1001/jama.2020.3786 (2020).
Basu A, Z. T., Inglima K, Woo K, Atie O, Yurasits L, See B, Aguero-Rosenfeld ME. Performance of the rapid Nucleic Acid Amplification by Abbott ID NOW COVID-19 in nasopharyngeal swabs transported in viral media and dry nasal swabs, in a New York City academic institution. bioRxiv, doi: doi:
https://doi.org/10.1101/2020.05.11.089896 (2020).
Priscila M. Kosaka, V. P., Montserrat Calleja, and Javier Tamayo. Ultrasensitive detection of HIV-1 p24 antigen by a hybrid nanomechanical-optoplasmonic platform with potential for detecting HIV-1 at first week after infection. PLOS One 12, e0171899 (2017).
Caroline Pereira Bittencourt Passaes, T. B., Jérémie Decalf, Annie David, Mathieu Angin, Valerie Monceaux, Michaela Muller-Trutwin, Nicolas Noel, Katia Bourdic, Olivier Lambotte, Matthew L. Albert, Darragh Duffy, Olivier Schwartz, Asier Sáez-Cirión. Ultrasensitive HIV-1 p24 Assay Detects Single Infected Cells and Differences in Reservoir Induction by Latency Reversal Agents. J Virol 91, e02296-02216 (2017).
Chen, H. et al. Quantitation of Femtomolar-Level Protein Biomarkers Using a Simple Microbubbling Digital Assay and Bright-Field Smartphone Imaging. Angewandte Chemie 131, 14060-14066, doi: 10.1002/ange.201906856 (2019).

1-56. (canceled)

57. A method, comprising:
contacting an analyte, a promoter tag, and an anchor,
the promoter tag being configured to bind to the analyte,
the promoter tag further comprising a reaction promoter,
the anchor being configured to bind to the analyte,
the contacting being performed under conditions such that the promoter tag binds with
the analyte and the anchor binds with the analyte so as to form a complex;
contacting the complex with a reaction substrate so as to evolve a reaction product; and
detecting at least some of the reaction product.

58. The method of claim 57, wherein the analyte is a coronavirus nucleocapsid or a coronavirus spike.

59. The method of claim 58, wherein the coronavirus is SARS-COV-2.

60. The method of claim 59, wherein the analyte is a nucleocapsid protein or a portion of a nucleocapsid protein of SARS-COV-2.

61. The method of claim 59, wherein the analyte is a spike protein or a portion of a spike protein of SARS-COV-2.

62. The method of claim 57, wherein the analyte is collected from a nasopharyngeal sample, a nasal sample, a urine sample, a stool sample, a saliva sample, another body fluid, or any combination thereof.

63. The method of claim 62, wherein the analyte is collected from a nasal sample.

64. The method of claim 62, wherein the analyte is collected from (a) a saliva sample or (b) another body fluid.

65. The method of claim 57, wherein the sample is taken from an individual (a) known or suspected to be post-infection with SARS-COV-2, (b) known or suspected to have received treatment for SARS-COV-2, (c) known or suspected to have received a vaccine for SARS-COV-2, or any combination of (a), (b), and (c).

66. The method of claim 57, wherein the promoter tag comprises an antibody complementary to the analyte, a nucleic acid complementary to the analyte, an aptamer complementary to the analyte, a nanobody complementary to the analyte, an affinity peptide complementary to the analyte, a molecular imprinting polymer complementary to the analyte, a ligand complementary to the analyte, a small molecule complementary to the analyte, a drug complementary to the analyte, or any combination thereof.

67. The method of claim 57, wherein the promoter tag comprises a catalyst portion.

68. The method of claim 67, wherein the catalyst portion comprises a metal, an enzyme, a metal oxide, a transition metal, a lanthanide, or any combination thereof.

69. The method of claim 68, wherein the metal comprises platinum and wherein the reaction substrate comprises hydrogen peroxide.

70. The method of claim 57, wherein the reaction product is a gas.

71. The method of claim 57, further comprising applying a gradient so as to direct the complex to a location, the anchor optionally being sensitive to the gradient.

72. The method of claim 57, wherein the detection comprises visual or optical detection.

73. The method of claim 72, wherein the detection is performed manually.

74. The method of claim 72, wherein the detection is performed in an automated fashion.

75. The method of claim 57, further comprising relating the detection of the at least some of the reaction product to a level of the analyte.

76. A kit, comprising:
a supply of a promoter tag configured to bind specifically to an analyte, a supply of an anchor configured to bind specifically to the analyte, the anchor optionally comprising a magnetizable material, and the promoter tag comprising a material configured to evolve a gaseous product when contacted with a reaction substrate under effective conditions.

77. The kit of claim 76, wherein the analyte is a coronavirus nucleocapsid or a coronavirus spike.

78. The kit of claim 77, wherein the coronavirus is SARS-COV-2.

79. The kit of claim 78, wherein the analyte is a nucleocapsid protein of SARS-COV-2

80. The kit of claim 78, wherein the analyte is a spike protein of SARS-COV-2.

81. The kit of claim 76, further comprising a translucent portion configured to permit observation of the gaseous product.

82. The kit of claim 76, wherein the kit is configured to engage with a portable computing device configured to observe the gaseous product.

83. A system, comprising:
an amount of a first promoter tag, the first promoter tag being configured to bind to a first analyte,
the first promoter tag further comprising a first reaction promoter,
an amount of a first anchor, the first anchor being configured to bind to the first analyte and the first anchor further comprising a ferromagnetic portion;
a substrate; and
a gradient source configured to exert a force on the ferromagnetic portion of the first anchor.

84. The system of claim 83, further comprising an amount of a second promoter tag,
the second promoter tag being configured to bind to a second analyte,
the second promoter tag further comprising a second reaction promoter,
an amount of a second anchor, the second anchor being configured to bind to the second analyte and the second anchor further comprising a ferromagnetic portion.

85. The system of claim 83, wherein the substrate comprises a plurality of depressions, and wherein the gradient source is configured to direct the first anchor to a location within a depression.

86. The system of claim 83, further comprising a detector configured to detect a product of a first reaction related to contact between the first reaction promoter and a reaction substrate.

87. The system of claim 83, further comprising a detector configured to detect a product of a second reaction related to contact between the second reaction promoter and a reaction substrate.

88. A method, comprising:
reacting a sample comprising an amount of an analyte with a promoter tag configured to bind specifically to the analyte under such conditions that the promoter tag binds to the analyte;
contacting the sample with an anchor under such conditions that the anchor binds specifically to the analyte,
the anchor optionally comprising a magnetizable material,
the reacting and contacting being performed so as to give rise to a complex that comprises the analyte, the promoter tag, and the anchor,
immobilizing the complex;
contacting the complex with a reaction substrate so as to evolve a gaseous reaction product;
detecting at least some of the gaseous reaction product; and
correlating detected reaction product with a level of the analyte in the sample.
